SEQUENCE LISTING (79 sequences)

SEQ ID NO: 1 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHDVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHEVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHRVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHMVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHLVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLWGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLDGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLKGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVFSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVWSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFYHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFFHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFWHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKWFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKAFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 254 aa, Artificial Sequence
  Synthetic polypeptide
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 18 - protein, 238 aa, Artificial Sequence
  Synthetic polypeptide
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 19 - protein, 15 aa, Artificial Sequence
  Synthetic polypeptide
GGGGSGGGGSGGGGS

SEQ ID NO: 20 - protein, 30 aa, Artificial Sequence
  Synthetic polypeptide
GGGGSGGGGSGGGGSGGGGSGGGGSGGGGS

SEQ ID NO: 21 - protein, 45 aa, Artificial Sequence
  Synthetic polypeptide
GGGGSGGGGSGGGGSGGGGSGGGGSGGGGSGGGGSGGGGSGGGGS

SEQ ID NO: 22 - protein, 60 aa, Artificial Sequence
  Synthetic polypeptide
GGGGSGGGGSGGGGSGGGGSGGGGSGGGGSGGGGSGGGGSGGGGSGGGGSGGGGSGGGGS

SEQ ID NO: 23 - protein, 75 aa, Artificial Sequence
  Synthetic polypeptide
GGGGSGGGGSGGGGSGGGGSGGGGSGGGGSGGGGSGGGGSGGGGSGGGGSGGGGSGGGGSGGGGSGGGGSGGGGS

SEQ ID NO: 24 - protein, 150 aa, Artificial Sequence
  Synthetic polypeptide
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 25 - protein, 225 aa, Artificial Sequence
  Synthetic polypeptide
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 26 - protein, 300 aa, Artificial Sequence
  Synthetic polypeptide
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 27 - protein, 451 aa, Artificial Sequence
  Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 451 aa, Artificial Sequence
  Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 451 aa, Artificial Sequence
  Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 451 aa, Artificial Sequence
  Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 31 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKAVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHDVTYLQNGKGRKYFHHNSDFYIPKATLKDSGSYFCRGLVGSDNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHDVTYLQNGKGRKYFHHNSDFYIPKATLKDSGSYFCRGLVGSDQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHEVTYLQNGKGRKYFHHNSDFYIPKATLKDSGSYFCRGLVGSENVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHEVTYLQNGKGRKYFHHNSDFYIPKATLKDSGSYFCRGLVGSEQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSDNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSENVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSDQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSEQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKAFHHNSDFYIPKATLKDSGSYFCRGLVGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHDVTYLQNGKGRKYFHHNSDFYIPKATLKDSGSYFCRGLVGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHEVTYLQNGKGRKYFHHNSDFYIPKATLKDSGSYFCRGLVGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHDVTYLQNGKGRKAFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHNVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHNVTYLQNGKGRKYFHHNSDFYIPKATLKDSGSYFCRGLVGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKAFHHNSDFYIPKATLKDSGSYFCRGLWGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHDVTYLQNGKGRKAFHHNSDFYIPKATLKDSGSYFCRGLVGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHEVTYLQNGKGRKAFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHEVTYLQNGKGRKAFHHNSDFYIPKATLKDSGSYFCRGLVGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHDVTYLQNGKGRKAFHHNSDFYIPKATLKDSGSYFCRGLWGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHDVTYLQNGKGRKAFHHNSDFYIPKATLKDSGSYFCRGLWGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLNGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLQGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLWGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHMVTYLQNGKGRKYFHHNSDFYIPKATLKDSGSYFCRGLVGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHLVTYLQNGKGRKYFHHNSDFYIPKATLKDSGSYFCRGLVGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKWFHHNSDFYIPKATLKDSGSYFCRGLVGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGKLLLQAPRWVFKEEDPIHLRCHSWKNTALHKVTYLQNGKGRKYFHHNSDFYIPKATLKDSGSYFCRGLDGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGRLLLQAPRWVFKEEDPIHLRCHSWKNTALHKVTYLQNGKGRKYFHHNSDFYIPKATLKDSGSYFCRGLDGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGFLLLQAPRWVFKEEDPIHLRCHSWKNTALHKVTYLQNGKGRKYFHHNSDFYIPKATLKDSGSYFCRGLDGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGVLLLQAPRWVFKEEDPIHLRCHSWKNTALHKVTYLQNGKGRKYFHHNSDFYIPKATLKDSGSYFCRGLDGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGKLLLQAPRWVFKEEDPIHLRCHSWKNTALHKVTYLQNGKGRKYFHHNSDFYIPKATLKDSGSYFCRGLDGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGRLLLQAPRWVFKEEDPIHLRCHSWKNTALHKVTYLQNGKGRKYFHHNSDFYIPKATLKDSGSYFCRGLDGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGFLLLQAPRWVFKEEDPIHLRCHSWKNTALHKVTYLQNGKGRKYFHHNSDFYIPKATLKDSGSYFCRGLDGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGVLLLQAPRWVFKEEDPIHLRCHSWKNTALHKVTYLQNGKGRKYFHHNSDFYIPKATLKDSGSYFCRGLDGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHMVTYLQNGKGRKYFHHNSDFYIPKATLKDSGSYFCRGLWGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHMVTYLQNGKGRKAFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHMVTYLQNGKGRKAFHHNSDFYIPKATLKDSGSYFCRGLWGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - protein, 239 aa, Artificial Sequence
  Synthetic polypeptide
MYRMQLLSCIALSLALVTNSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 471 aa, Artificial Sequence
  Synthetic polypeptide
MYRMQLLSCIALSLALVTNSEVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 72 - protein, 238 aa, Artificial Sequence
  Synthetic polypeptide
MYRMQLLSCIALSLALVTNSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 467 aa, Artificial Sequence
  Synthetic polypeptide
MYRMQLLSCIALSLALVTNSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 467 aa, Artificial Sequence
  Synthetic polypeptide
MYRMQLLSCIALSLALVTNSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 234 aa, Artificial Sequence
  Synthetic polypeptide
MYRMQLLSCIALSLALVTNSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 471 aa, Artificial Sequence
  Synthetic polypeptide
MYRMQLLSCIALSLALVTNSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPKVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 471 aa, Artificial Sequence
  Synthetic polypeptide
MYRMQLLSCIALSLALVTNSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPKVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 470 aa, Artificial Sequence
  Synthetic polypeptide
MYRMQLLSCIALSLALVTNSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 300 aa, Artificial sequence; identical to SEQ ID NO: 26
  Synthetic polypeptide
  MISC_FEATURE (16)..(300): Xaa may be absent

(19) United States
(12) Patent Application Publication
(10) Pub. No.: US 2019/0112349 A1
(43) Pub. Date: Apr. 18, 2019

(54) MODIFIED CHIMERIC RECEPTORS AND USES THEREOF IN IMMUNE THERAPY

(71) Applicant: Unum Therapeutics Inc., Cambridge, MA (US)

(72) Inventors: Kathleen McGinness, Cambridge, MA (US); Charles Wilson, Cambridge, MA (US); Seth Ettenberg, Cambridge, MA (US); Heather Huet, Cambridge, MA (US)

(73) Assignee: Unum Therapeutics Inc., Cambridge, MA (US)

(21) Appl. No.: 16/085,991

(22) PCT Filed: Mar. 17, 2017
(86) PCT No.: PCT/US17/23064

§ 371 Date: Sep. 17, 2018

Related U.S. Application Data
(60) Provisional application No. 62/310,316, filed on Mar. 18, 2016.

(51) Int. Cl. C07K 14/735; A61K 35/17; A61K 38/17; A61K 39/395; C07K 16/28

(52) U.S. Cl. CPC C07K 14/70535; A61K 35/17; A61K 38/1774; A61K 39/39541; C07K 16/283; C07K 16/2863; A61K 2039/505; C07K 2319/03; C07K 2319/02; C07K 2317/92; C07K 2317/73; C07K 2317/41; C07K 2319/01

(57) ABSTRACT

Disclosed herein are chimeric receptors that comprise an extracellular domain and a cytoplasmic signaling domain, wherein the extracellular domain has a reduced binding activity to a wild-type Fc fragment; nucleic acids encoding such chimeric receptors, and immune cells expressing the chimeric receptors. Also disclosed are methods of using the chimeric receptors to enhance the efficacy of antibody-based immunotherapy, such as cancer immunotherapy.

### CROSS REFERENCE TO RELATED APPLICATIONS

This application claims the benefit of U.S. Provisional Application No. 62/310,316, filed Mar. 18, 2016, under 35 U.S.C. § 119, the entire content of which is herein incorporated by reference.

### BACKGROUND OF DISCLOSURE

Cancer immunotherapy, including cell-based therapy, antibody therapy and cytokine therapy, is used to provoke immune attack of tumor cells while sparing normal tissues. It is a promising option for treating various types of cancer because of its potential to evade genetic and cellular mechanisms of drug resistance and to avoid many of the toxicities observed with traditional chemotherapies. T-lymphocytes can exert major anti-tumor effects as demonstrated by results of allogeneic hematopoietic stem cell transplantation (HSCT) for hematologic malignancies, where T-cell-mediated graft-versus-host disease (GvHD) is inversely associated with disease recurrence, and immunosuppression withdrawal or infusion of donor lymphocytes can contain relapse. Weiden et al., N Engl J Med. 1979; 300(19):1068-1073; Porter et al., N Engl J Med. 1994; 330(2):100-106; Kolb et al., Blood. 1995; 86(5):2041-2050; Slavin et al., Blood. 1996; 87(6):2195-2204; and Appelbaum, Nature. 2001; 411(6835):385-389.

Cell-based therapy may involve cytotoxic T cells having reactivity skewed toward cancer cells. Eshhar et al., Proc. Natl. Acad. Sci. U.S.A; 1993; 90(2):720-724; Geiger et al., J Immunol. 1999; 162(10):5931-5939; Brentjens et al., Nat. Med. 2003; 9(3):279-286; Cooper et al., Blood. 2003; 101(4):1637-1644; and Imai et al., Leukemia. 2004; 18:676-684. One approach is to express a chimeric antigen receptor having an antigen-binding domain fused to one or more T cell activation signaling domains. Binding of a cancer antigen via the antigen-binding domain results in T cell activation and triggers cytotoxicity. Recent results of clinical trials with infusions of chimeric receptor-expressing autologous T lymphocytes provided compelling evidence of their clinical potential. Pule et al., Nat. Med. 2008; 14(11):1264-1270; Porter et al., N Engl J Med; 2011; 25; 365(8):725-733; Brentjens et al., Blood. 2011; 118(18):4817-4828; Till et al., Blood. 2012; 119(17):3940-3950; Kochenderfer et al., Blood. 2012; 119(12):2709-2720; and Brentjens et al., Sci Transl Med. 2013; 5(177):177ra138.

Antibody-based immunotherapies, such as monoclonal antibodies, antibody-fusion proteins, and antibody drug conjugates (ADCs) are used to treat a wide variety of diseases, including many types of cancer. Such therapies may depend on recognition of cell surface molecules that are differentially expressed on cells for which elimination is desired (e.g., target cells such as cancer cells) relative to normal cells (e.g., non-cancer cells). Binding of an antibody-based immunotherapy to a cancer cell can lead to cancer cell death via various mechanisms, e.g., antibody-dependent cell-mediated cytotoxicity (ADCC), complement-dependent cytotoxicity (CDC), or direct cytotoxic activity of the payload from an antibody-drug conjugate (ADC).

### SUMMARY OF DISCLOSURE

The present disclosure is based on the design of antibody-coupled T cell receptor (ACTR) variants comprising a mutated extracellular ligand-binding domain of an Fc receptor, which leads to reduced binding to a wild-type Fc fragment as defined herein. The ACTR variants described herein can bind to antibodies containing amino acid mutations in the Fc fragment and/or having an afucosylated Fc fragment. Immune cells expressing such an ACTR variant would enhance the efficacy of antibody-based immunotherapies, for example by reducing or eliminating binding competition from endogenous antibodies or other proteins comprising a wild-type Fc fragment, and/or reducing potential undesired side effects of antibody-based immunotherapies, such as acute or chronic autoimmunity.

Accordingly, one aspect of the present disclosure features a chimeric receptor, comprising: (a) an extracellular domain, which is either a mutated extracellular ligand-binding domain of an Fc receptor, or a single chain antibody fragment; and (b) a cytoplasmic signaling domain (e.g., from a CD3 receptor). In some embodiments, the extracellular domain in the chimeric receptor is a mutated extracellular ligand-binding domain of an Fc receptor. As compared with the wild-type counterpart, the mutated extracellular ligand-binding domain of the Fc receptor comprises a mutation at one or more residues involved in Fc receptor/Fc interaction such that the mutated extracellular ligand-binding domain of the Fc receptor has a reduced binding activity to a wild-type Fc fragment relative to the wild-type Fc receptor counterpart. In some embodiments, the extracellular domain in the chimeric receptor is a single chain antibody fragment, which binds preferentially to a mutated Fc fragment as relative to its wild-type counterpart.

In some embodiments, the chimeric receptor further comprises one or more of the following domains: a transmembrane domain; one or more co-stimulatory signaling domains; and a hinge domain. In some examples, the chimeric receptor comprises, from N terminus to C terminus, (a) the extracellular domain; (b) the transmembrane domain; (c) the one or more co-stimulatory signaling domains; and (d) the cytoplasmic signaling domain.

In some examples, the chimeric receptor further comprises the hinge domain, which is located between (a) and (b). Alternatively or in addition, the chimeric receptor further comprises a signal peptide.

In some embodiments, the extracellular domain of the chimeric receptor is a mutated extracellular-ligand binding domain derived from an Fcγ receptor (FcγR), e.g., CD16A, CD16B, CD64A, CD64B, CD64C, CD32A, or CD32B. In some examples, the one or more residues where the mutation occurs are located in the D2 domain of the extracellular ligand-binding domain of the FcγR.

In some embodiments, the extracellular domain of the chimeric receptor is a mutated extracellular-ligand binding domain of CD16A (e.g., SEQ ID NO:18). In some examples, the mutation is an amino acid substitution at one or more positions corresponding to 92, 122, 134, 136, 160, 161, 163, and 164 in SEQ ID NO: 18. In some instances, the mutated extracellular ligand-binding domain of the Fc receptor comprises amino acid substitutions at two or more positions selected from the group consisting of the positions corresponding to 92, 122, 134, 136, 160, 161, 163, and 164 in SEQ ID NO: 18.

In some embodiments, the mutated extracellular ligand-binding domain of the Fc receptor comprises an amino acid substitution at a position corresponding to 160 in SEQ ID NO:18, at a position corresponding to 134 in SEQ ID NO:18, at a position corresponding to 122 in SEQ ID NO: 18, at a position corresponding to 164 in SEQ ID NO:18, or a combination thereof. In some examples, the mutated extracellular ligand-binding domain of the Fc receptor comprises a V to Q or a V to W amino acid substitution at the position corresponding to 160 in SEQ ID NO: 18. In other examples, the mutated extracellular ligand-binding domain of the Fc receptor comprises a Y to A amino acid substitution at the position corresponding to 134 in SEQ ID NO: 18. In yet other examples, the mutated extracellular ligand-binding domain of the Fc receptor comprises a K to L amino acid substitution at the position corresponding to 122 in SEQ ID NO: 18; or an N to Q amino acid substitution at the position corresponding to 164 in SEQ ID NO: 18. In some specific examples, the mutated extracellular ligand-binding domain of the Fc receptor is a mutated CD16A of mutant V160Q, mutant V160W, mutant Y134A, mutant K122L, and mutant Y134A/N164Q.

In some specific examples, the chimeric receptor comprises an amino acid sequence of any one of SEQ ID NO: 1-16 and 33-69.

Another aspect of the present disclosure features a nucleic acid comprising a nucleotide sequence encoding any of the chimeric receptors described herein; vectors (e.g., expression vectors) comprising the nucleic acid; and host cells (e.g., immune cells such as natural killer (NK) cells, macrophages, neutrophils, eosinophils, and T cells) expressing any of the chimeric receptors described herein. In some embodiments, the vector is a viral vector (e.g., a retroviral vector, a lentiviral vector, or an adeno-associated viral vector).

In some embodiments, the immune cell as described herein is a T lymphocyte or an NK cell, both of which may be activated and/or expanded ex vivo. In some instances, the T lymphocyte may be engineered (e.g., having reduced or eliminated expression of T cell receptors) to reduce its graft versus host effects when given to a subject.

In yet another aspect, described herein are pharmaceutical compositions (or kits as described herein) that comprise any of the immune cells that express the chimeric receptor described herein and a pharmaceutically acceptable carrier or excipient. In some embodiments, the pharmaceutical composition further comprises an Fc-containing polypeptide that binds to the chimeric receptor described herein. In some embodiments, the Fc-containing polypeptide is an antibody. In certain embodiments, the Fc-containing polypeptide is afucosylated in its Fc domain.

In some embodiments, the Fc-containing polypeptide comprises one or more mutations in the Fc region such that the mutated Fc-containing polypeptide has an enhanced binding activity to the chimeric receptor as compared with its wild-type counterpart. In some embodiments, the Fc-containing polypeptide is an antibody containing an amino acid substitution at one or more positions corresponding to S239, F243, R292, S298, Y300, V305, A330, I332, E333, K334, and P396 of a wild-type antibody. Unless indicated otherwise, the numbering system referring to positions in an antibody (e.g., in the Fc domain thereof) used herein is according to the EU index. In some embodiments, the amino acid substitution is S239D, S239K, F243L, R292P, S298A, Y300L, V305I, A330L, I332E, I332D, E333A, K334A, P396L, or a combination thereof. In certain embodiments, the immune cell expresses any chimeric receptor described herein, which comprises an amino acid substitution at one or more positions corresponding to 122, 134, 160, and 164 in SEQ ID NO: 18. Such Fc-containing polypeptides may be afucosylated in its Fc domain.

In some embodiments, the immune cell expresses a chimeric receptor, which comprises the CD16A mutant V160Q, the CD16A mutant V160W, or the CD16A mutant K122L, and the Fc-containing polypeptide can be an afucosylated full-length antibody. In some embodiments, the immune cell expresses a chimeric receptor which comprises an amino acid substitution at one or more positions corresponding to 122, 134, 160, and 164 in SEQ ID NO: 18, and the Fc-containing polypeptide can be an antibody containing an amino acid substitution at one or more positions corresponding to S239, F243, R292, S298, Y300, V305, A330, I332, E333, K334, and P396 of a wild-type antibody.

In some embodiments, the immune cell expresses a chimeric receptor, which comprises the CD16A mutant Y134A/N164Q, and the Fc-containing polypeptide can be an antibody containing (i) S239D, A330L, and I332E substitutions, or (ii) S239D and I332E substitutions as compared with the wild-type counterpart. In certain embodiments, the immune cell expresses a chimeric receptor, which comprises the CD16A mutant Y134A, and the Fc-containing polypeptide can be an antibody containing (i) S239D, A330L, and I332E substitutions, or (ii) S239D and I332E substitutions as compared with the wild-type counterpart.

In some embodiments, the immune cell expresses a chimeric receptor, which comprises the CD16A mutant K122L, and the Fc-containing polypeptide can be an antibody containing (i) S298A, E333A, and K334A substitutions, or (ii) F243L, R292P, Y300L, V305I, and P396L substitutions as compared with the wild-type counterpart. In some embodiments, the immune cell expresses a chimeric receptor, which comprises the CD16A mutant V160Q, and the Fc-containing polypeptide can be an antibody containing (i) S298A, E333A, and K334A substitutions, (ii) S239D, A330L, and I332E substitutions, (iii) S239D and I332E substitutions, or (iv) F243L, R292P, Y300L, V305I, and P396L substitutions as compared with the wild-type counterpart. In some embodiments, the immune cell expresses a chimeric receptor, which comprises the CD16A mutant V160W, and the Fc-containing polypeptide can be an antibody containing (i) S298A, E333A, and K334A substitutions, (ii) S239D, A330L, and I332E substitutions, (iii) S239D and I332E substitutions, or (iv) F243L, R292P, Y300L, V305I, and P396L substitutions as compared with the wild-type counterpart.

Another aspect of the the present disclosure features a kit for an antibody-coupled T cell receptor (ACTR) immunotherapy, comprising any of the immune cells described herein expressing any of the the chimeric receptor described herein; and an Fc-containing polypeptide that binds the chimeric receptor. In some embodiments, the kit comprises any of the specific combinations of the immune cells and Fc-containing polypeptides as described herein.

Another aspect of the the present disclosure features a method for enhancing the efficacy of an antibody-based immunotherapy, the method comprising administering to a subject in need thereof (i) a therapeutically effective amount of any immune cell described herein that express any of the chimeric receptors described herein, and (ii) a therapeutically effective amount of an Fc-containing polypeptide that binds the chimeric receptor as also described herein, for example, the specific immune cell/Fc-containing polypeptide combinations described herein

In some embodiments, the subject has a cancer, including, but not limited to, carcinoma, lymphoma, sarcoma, blastoma, and leukemia. In some examples, the cancer can be a cancer of B-cell origin (e.g., B-lineage acute lymphoblastic leukemia, B-cell chronic lymphocytic leukemia, and B-cell non-Hodgkin's lymphoma), breast cancer, gastric cancer, neuroblastoma, osteosarcoma, lung cancer, melanoma, prostate cancer, colon cancer, renal cell carcinoma, ovarian cancer, rhabdomyosarcoma, leukemia, and Hodgkin's lymphoma.

In some embodiments, the immune cell is a T lymphocyte or an NK cell, which may be autologous (obtained from the same patient) or allogenic (obtained from a donor of the same species as the recipient). In some embodiments, prior to the administration step, the autologous T lymphocyte or autologous NK cells are activated and/or expanded ex vivo. In some embodiments, the allogeneic T lymphocyte is engineered to reduce graft-versus-host effects or host-versus-graft effects. In some embodiments, the endogenous T cell receptor of the allogeneic T lymphocyte has been inhibited or eliminated. In some embodiments, prior to the administration step, the allogenic T lymphocyte or allogenic NK cell are activated and/or expanded ex vivo.

In some embodiments, the Fc-containing polypeptide is afucosylated in its Fc domain. In some embodiments, the Fc-containing polypeptide is a therapeutic antibody. In some embodiments, the therapeutic antibody is selected from the group consisting of Rituximab, Trastuzumab, hu14.18K322A, Epratuzumab, Cetuximab, and Labetuzumab.

Another aspect of the the present disclosure features a method for preparing an immune cell expressing a chimeric receptor, comprising (i) providing a population of any of the immune cells described herein; (ii) introducing into the immune cells a nucleic acid encoding any chimeric receptor described herein; and (iii) culturing the immune cells under conditions allowing for expression of the chimeric receptor.

In some embodiments, the method further comprises (iv) activating the immune cells expressing the chimeric receptor. In some embodiments, the immune cells comprise T lymphocytes, which are activated in the presence of one or more of anti-CD3 antibody, anti-CD28 antibody, IL-2, IL-7, IL-15, and phytohemoagglutinin. In certain embodiments, the immune cells comprise NK cell, which are activated in the presence of one or more of 4-1BB ligand, anti-4-1BB antibody, IL-15 protein, IL-15 receptor antibody, IL-2 protein, IL-21 protein, K562 cell line.

In some embodiments, the population of immune cells is derived from peripheral blood mononuclear cells (PBMC). In some embodiments, the immune cells comprise T lymphocytes and/or NK cells. In some embodiments, the immune cells are derived from a human cancer patient, such as from the bone marrow or a tumor of a human cancer patient. In some embodiments, the immune cells are derived from a healthy donor.

In some embodiments, the nucleic acid encoding the chimeric receptor is inserted into a vector, such as a viral vector or a transposon, which may be introduced into a suitable immune cell by lentiviral transduction, retroviral transduction, adeno-associated viral transduction, DNA electroporation, or transposon electroporation. In other embodiments, the nucleic acid encoding the chimeric receptor may be an RNA molecule, which may be introduced into the immune cell via RNA electroporation.

Also within the scope of the present disclosure are any of the chimeric receptors (e.g., an ACTR variant) that comprise (a) an extracellular domain as described herein (e.g., an scFv binding to a mutated Fc fragment or a mutated extracellular ligand-binding domain of an Fc receptor); and (b) a cytoplasmic signaling domain, as described herein, host cells (such as immune cells as those described herein) expressing such ACTR variants, or pharmaceutical compositions comprising the host cells for use in treating cancer (e.g., a carcinoma, a lymophoma, a sarcoma, a blastoma, or a leukemia), as well as the use of the chimeric receptors, the host cells expressing such, or the pharmaceutical compositions comprising such in manufacturing a medicament for use in cancer treatment.

The details of one of more embodiments of the disclosure are set forth in the description below. Other features or advantages of the present disclosure will be apparent from the detailed description of several embodiments and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 is a cartoon showing the basic strategy to generate variant ACTRs with reduced engagement of endogenously-expressed antibodies and preferential engagement of variant antibodies. Endogenous antibodies and therapeutic antibodies engage effectively with ACTR. Variant ACTRs as described herein reduce or eliminate interaction with endogenous antibodies. Variant antibodies as described herein can productively engage with the ACTR variant.

FIG. 2 is a graph showing binding of rituximab and afucosylated rituximab to Jurkat cells expressing ACTR variants. The geometric mean (GM) of fluorescence, which is a measure of antibody binding to ACTR-expressing cells, is plotted as a function of antibody concentration. Cells expressing ACTR variant 1 bind to rituximab while those expressing ACTR variant 26 do not bind to rituximab at the antibody concentrations tested. The afucosylated antibody shows binding to ACTR variant 26, demonstrating that absence of the fucose restores binding to this ACTR variant.

FIG. 3 is a graph showing activation of Jurkat NFAT reporter cells expressing ACTR variants in the presence of rituximab (closed symbols) or afucosylated rituximab (open symbols). Luminescence is a measure of luciferase upregulation, which is regulated by upregulation of NFAT upon cell activation. Jurkat NFAT reporter cells expressing ACTR variant 1 show concentration-dependent activation in the presence of rituximab. Jurkat NFAT reporter cells expressing ACTR variant 26 show concentration-dependent activation in the presence of afucosylated rituximab but not in the presence of rituximab.

FIG. 4 is a set of graphs showing the expression and activity levels of example ACTR variants expressed on T cells. A: T-cells transduced with retrovirus encoding ACTR variant 1 (left panel) and ACTR variant 26 (right panel) express similar levels and amounts of ACTR. (B) T-cells expressing ACTR variant 1, ACTR variant 26, or mock were incubated with luciferase-expressing target Daudi cells in the presence of increasing concentrations of rituximab or afucosylated rituximab for 44 hr. Luciferase activity was measured and percent cytotoxicity was calculated relative to reactions without antibody. Percent cytotoxicity is plotted as a function and antibody concentration. ACTR variant 1-expressing T-cells mediate antibody-dependent cytotoxicity in the presence of rituximab, whereas ACTR variant 26-expressing T-cells mediate antibody-dependent cytotoxicity in the presence of afucosylated rituximab. No cytotoxicity was observed with ACTR variant 26 T-cells in the presence of rituximab or with mock T-cells in the presence of afucosylated rituximab.

FIG. 5 is a graph showing activation of Jurkat NFAT reporter cells expressing ACTR variant 26 in the presence of rituximab (black closed circles), afucosylated rituximab (black open circles), rituximab heavy chain Fc variant 174 (black closed squares), rituximab heavy chain Fc variant 175 (black open squares), rituximab heavy chain Fc variant 176 (gray closed triangles), rituximab heavy chain Fc variant 177 (gray open triangles). Luminescence is a measure of luciferase upregulation, which is regulated by upregulation of NFAT upon cell activation. Jurkat NFAT reporter cells expressing ACTR variant 25 show concentration-dependent activation in the presence of afucosylated rituximab and rituximab heavy chain Fc variants, but not in the presence of rituximab.

FIG. 6 is a series of graphs showing activation of untransduced T-cells (mock) and T-cells bearing ACTR variants SEQ ID NO: 1 and SEQ ID NO: 16 in the presence of Daudi target cells and rituximab. Activation was measured by evaluating (A) CD25 MFI, (B) CD69 MFI, and (C) IFNγ production as a function of rituximab concentration.

FIG. 7 is a series of graphs showing activation of untransduced T-cells (mock) and T-cells bearing ACTR variants SEQ ID NO: 1 and SEQ ID NO: 41 in the presence of Daudi target cells and rituximab. Activation was measured by evaluating (A) CD25 expression (MFI), (B) CD69 expression (MFI), and (C) IFNγ production as a function of rituximab concentration.

FIG. 8 is a series of graphs showing activation of untransduced T-cells (mock) and T-cells bearing ACTR variants SEQ ID NO: 1 and SEQ ID NO: 54 in the presence of Daudi target cells and rituximab. Activation was measured by evaluating (A) CD25 expression (MFI), (B) CD69 expression (MFI), and (C) IFNγ production as a function of rituximab concentration.

FIG. 9 is a series of graphs showing activation of untransduced T-cells (mock) and T-cells bearing ACTR variants SEQ ID NO: 1 and SEQ ID NO: 6 in the presence of Daudi target cells and rituximab. Activation was measured by evaluating (A) CD25 expression (MFI), (B) CD69 expression (MFI), and (C) IFNγ production as a function of rituximab concentration.

FIG. 10 is a series of graphs showing activation of untransduced T-cells (mock) and T-cells bearing ACTR variants SEQ ID NO: 1 and SEQ ID NO: 7 in the presence of Daudi target cells and rituximab. Activation was measured by evaluating (A) CD25 expression (MFI), (B) CD69 expression (MFI), and (C) IFNγ production as a function of rituximab concentration.

FIG. 11 is a series of graphs showing activation ACTR-expressing Jurkat cells with an NFAT reporter in the presence of BCMA-expressing H929 target cells and anti-BCMA antibodies. The non-zero dotted line on each graph indicates the half-maximal luminescence value of the highest luminescence value measured with the same ACTR variant and the afucosylated Fc-enhanced antibody. Results are shown for: (A) ACTR variant SEQ ID NO: 1; (B) ACTR variant SEQ ID NO: 2; (C) ACTR variant SEQ ID NO: 6; (D) ACTR variant SEQ ID NO: 7; (E) ACTR variant SEQ ID NO: 16; and (F) ACTR variant SEQ ID NO: 54 with wild-type, afucosylated, Fc-enhanced (heavy chain SEQ ID NO: 74), and afucosylated Fc-enhanced (heavy chain SEQ ID NO: 74) antibodies.

FIG. 12 is a set of graphs showing activation ACTR-expressing Jurkat cells with an NFAT reporter in the presence of CD19-expressing Raji target cells and an Fc-enhanced anti-CD19 antibody. Results are shown for: (A) ACTR variants SEQ ID 1, 16, 41, 54, and 58; and (B) ACTR variants SEQ ID 2, 5, 6, 15, 39, 40, and 43.

FIG. 13 is a series of graphs showing activity of ACTR-expressing T-cells in the presence of Her2-expressing SKBR3 cells and trastuzumab or afucosylated trastuzumab. Results are shown for: (A) cytotoxicity using ACTR variants SEQ ID NO: 5 and SEQ ID NO: 6 and wild-type ACTR SEQ ID NO: 1; (B) cytotoxicity using ACTR variant SEQ ID NO: 7 and wild-type ACTR SEQ ID NO: 1; and (C) IFNγ release for ACTR variants SEQ ID NOs: 5, 6, and 7 and wild-type ACTR SEQ ID NO: 1.

FIG. 14 is a series of graphs showing of IFNγ released from ACTR-bearing T-cells in the presence of Daudi target cells and rituximab or afucosylated rituximab. Results are shown for: (A) ACTR variants SEQ ID NOs: 5 and 6 and wild-type ACTR SEQ ID NO: 1; (B) ACTR variants SEQ ID NOs: 7 and 15 and wild-type ACTR SEQ ID NO: 1; and (C) ACTR variant SEQ ID NO: 54 and wild-type ACTR SEQ ID NO: 1.

FIG. 15 is a series of graphs showing IL2 release from ACTR-bearing T-cells in the presence of Daudi target cells and rituximab or afucosylated rituximab. Results are shown for: (A) ACTR variants SEQ ID NOs: 5 and 6 and wild-type ACTR SEQ ID NO: 1; (B) ACTR variants SEQ ID NOs: 7 and 15 and wild-type ACTR SEQ ID NO: 1; and (C) ACTR variant SEQ ID NO: 54 and wild-type ACTR SEQ ID NO: 1.

FIG. 16 is a series of graphs showing Daudi target cell toxicity in the presence of ACTR-bearing T-cells and rituximab or afucosylated rituximab. Results are shown for: (A) ACTR variant SEQ ID NO: 54 and wild-type ACTR SEQ ID NO: 1; (B) ACTR variant SEQ ID NO: 16 and wild-type ACTR SEQ ID NO: 1; and (C) ACTR variant SEQ ID NO: 41 and wild-type ACTR SEQ ID NO: 1.

FIG. 17 is a set of graphs showing (A) IFNγ and (B) IL2 release from ACTR-bearing T-cells in the presence of Daudi target cells and rituximab or afucosylated rituximab. Results are shown for ACTR variants SEQ ID NOs: 16 and 41 and wild-type ACTR (SEQ ID NO: 1).

FIG. 18 is graph showing untransduced (mock) or ACTR-bearing T-cell proliferation in the presence of Raji target cells and rituximab or afucosylated rituximab. The CD3-positive cell count was evaluated by flow cytometry as a measure of an increase in T-cells, which is reflective of proliferation. Results are shown for ACTR variants SEQ ID NOs: 16 and 54 and wild-type ACTR (SEQ ID NO: 1).

FIG. 19 is a set of graphs showing IFNγ release from ACTR-bearing T-cells in the presence of Daudi target cells and rituximab or Fc-enhanced variants of rituximab. Results are shown for (A) ACTR variant SEQ ID NO: 16 and wild-type ACTR (SEQ ID NO: 1); and (B) ACTR variant SEQ ID NO: 41 and wild-type ACTR (SEQ ID NO: 1).

FIG. 20 is a set of graphs showing IL2 release from ACTR-bearing T-cells in the presence of Daudi target cells and rituximab or Fc-enhanced variants of rituximab. Results are shown for (A) ACTR variant SEQ ID NO: 16 and wild-type ACTR (SEQ ID NO: 1); and (B) ACTR variant SEQ ID NO: 41 and wild-type ACTR (SEQ ID NO: 1).

FIG. 21 is a series of graphs showing IL2 release from ACTR-bearing T-cells in the presence of Daudi target cells and rituximab or Fc-enhanced variants of rituximab, with or without fucose. Results are shown for: (A) ACTR variant SEQ ID NO: 16 and rituximab or rituximab with heavy chain SEQ ID NO: 28 or 29; (B) ACTR variant SEQ ID NO: 7 and rituximab or rituximab with heavy chain SEQ ID NO: 27 or 28; and (C) ACTR variant SEQ ID NO: 7 and rituximab or rituximab with heavy chain SEQ ID NO: 29 or 30.

FIG. 22 is a graph showing IL2 release from ACTR-bearing T-cells expressing ACTR variant SEQ ID NO: 54 in the presence of Daudi target cells and Fc-enhanced variants of rituximab (SEQ ID NOs: 27 and 28), with or without fucose.

FIG. 23 is a graph showing Daudi target cell toxicity in the presence of untransduced (mock) or ACTR-bearing T-cells and rituximab or afucosylated rituximab with heavy chain variant SEQ ID 29. Results are shown for ACTR variants of SEQ ID NOs: 16, 41, and 54 as well as wild-type ACTR (SEQ ID NO: 1).

FIG. 24 is a series of graphs showing ACTR-bearing T-cell proliferation in the presence of Raji target cells and rituximab or Fc-enhanced rituximab variants with or without fucose. The CD3-positive cell count was evaluated by flow cytometry as a measure of an increase in T-cells, which is reflective of proliferation. Results are shown for (A) ACTR variant SEQ ID NO: 16 and fucosylated or afucosylated rituximab or rituximab with heavy chain SEQ ID NO: 28 or 29; (B) ACTR variant SEQ ID NO: 41 and fucosylated or afucosylated rituximab or rituximab with heavy chain SEQ ID NO: 28 or 29; (C) ACTR variant SEQ ID NO: 54 and fucosylated or afucosylated rituximab or rituximab with heavy chain SEQ ID NO: 27 or 28; and (D) ACTR variant SEQ ID NO: 54 and wild-type rituximab or rituximab with heavy chain SEQ ID NO: 29 or 30. Results are also shown using wild-type ACTR (SEQ ID NO: 1).

FIG. 25 is a set of graphs showing activation of T-cells bearing ACTR variants SEQ ID NO: 16 and SEQ ID NO: 54 in the presence of Daudi target cells and rituximab, afucosylated rituximab, and afucosylated rituximab with heavy chain variant SEQ ID NO: 29. Results for (A) CD25 expression and (B) CD69 expression are shown.

FIG. 26 is a series of graphs showing activation of ACTR-expressing Jurkat cells with an NFAT reporter in the presence of CD20-expressing Daudi target cells and rituximab, afucosylated rituximab, or Fc-enhanced versions of rituximab. Results are shown for (A) ACTR variant SEQ ID NO: 6; (B) ACTR variant SEQ ID NO: 7; (C) ACTR variant SEQ ID NO: 16; (D) ACTR variant SEQ ID NO: 41; and (E) ACTR variant SEQ ID NO: 54.

FIG. 27 is a series of graphs showing activation of ACTR-expressing Jurkat cells with an NFAT reporter in the presence of CD20-expressing Daudi target cells and rituximab, afucosylated rituximab, rituximab with heavy chain variant SEQ ID NO: 28, and afucosylated rituximab with heavy chain variant SEQ ID NO: 28. Results are shown for: (A) ACTR variant SEQ ID NO: 5; (B) ACTR variant SEQ ID NO: 15; (C) ACTR variant SEQ ID NO: 16; and (D) ACTR variant SEQ ID NO: 54.

FIG. 28 is a series of graphs showing activation of ACTR-expressing Jurkat cells with an NFAT reporter in the presence of CD20-expressing Daudi target cells and rituximab, afucosylated rituximab, rituximab with heavy chain variant SEQ ID 28, and afucosylated rituximab with heavy chain variant SEQ ID 28. Results are shown for: (A) ACTR variant SEQ ID NO: 6; (B) ACTR variant SEQ ID NO: 41; and (C) ACTR variant SEQ ID: 58.

FIG. 29 is a series of graphs showing activation of ACTR-expressing Jurkat cells with an NFAT reporter in the presence of CD20-expressing Daudi target cells and rituximab with heavy chain variant SEQ ID 29, rituximab with heavy chain variant SEQ ID 76, and rituximab with heavy chain variant SEQ ID 77. Results are shown for: (A) ACTR variant SEQ ID 2; (B) ACTR variant SEQ ID 42; and (C) ACTR variant SEQ ID 42.

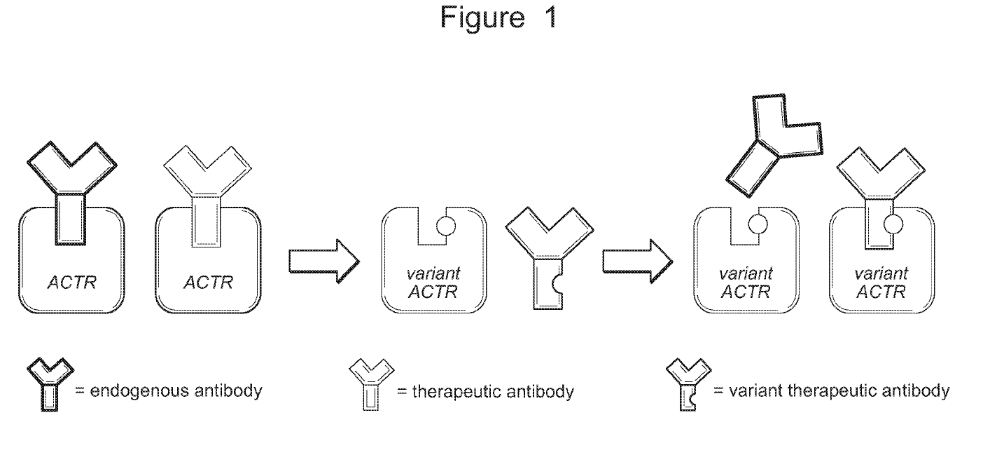

### DETAILED DESCRIPTION OF DISCLOSURE

Antibody-based immunotherapies are used to treat a wide variety of diseases, including many types of cancer. Such a therapy often depends on recognition of cell surface molecules that are differentially expressed on cells for which elimination is desired (e.g., target cells such as cancer cells) relative to normal cells (e.g., non-cancer cells) (Weiner et al. Cell (2012) 148(6): 1081-1084). Several antibody-based immunotherapies have been shown in vitro to facilitate antibody-dependent cell-mediated cytotoxicity of target cells (e.g. cancer cells), and for some it is generally considered that this is the mechanism of action in vivo, as well. ADCC is a cell-mediated innate immune mechanism whereby an effector cell of the immune system, such as natural killer (NK) cells, T cells, monocyte cells, macrophages, or eosinophils, actively lyses target cells (e.g., cancer cells) recognized by specific antibodies.

Described herein are ACTR variants that have been designed to have reduced binding activity to molecules (e.g., immunoglobulin molecules) having a wild-type Fc fragment, which refers to an Fc fragment having an amino acid sequence of a naturally-occurring Fc fragment and is fucosylated at a glycosylation site therein. A wild-type Fc fragment may be a portion of a molecule produced in a subject endogenously, for example, an endogenous antibody. Alternatively, it may be a portion of a recombinantly produced molecule such as an antibody, which has the same amino acid sequence of a naturally-occurring Fc fragment and substantially the same post-translational modification patterns such as glycosylation.

The ACTR variants are capable of binding to molecules containing Fc fragments that are afucosylated, contain mutations in the regions involved in Fc receptor/Fc interaction, or both. The ACTR variants and immune cells expressing such would confer a number of advantages in antibody-based immunotherapies. For example, use of the ACTR variants or immune cells expressing the variants may increase the efficacy of antibody-based immunotherapies and/or reduce undesired side effects. The ACTR variants may comprise a mutated extracellular ligand-binding fragment of an Fc receptor, which contains mutation at one or more residues involved in Fc receptor/Fc interaction, thereby reducing or eliminating competitive binding of the ACTR variants by endogenous antibodies or other molecules containing a wild-type Fc domain. Furthermore, by reducing or preventing binding of immune cells expressing the ACTR variants to proteins containing wild-type Fc fragments, for example endogenous antibodies that are bound to or may bind to non-target cells (e.g., normal tissue cells), ADCC of non-target cells and autoimmune responses may also be reduced.

Also disclosed herein are methods of using the ACTR variants and immune cells expressing the variants in the presence of a molecule comprising an Fc-containing polypeptide such as an antibody that is capable of binding to the ACTR variant. The Fc domain of such a polypeptide is modified to allow for interaction with the ACTR variant described herein. For example, the Fc domain may be afucosylated and/or mutated at one or more residues involved in Fc/Fc receptor interaction. The methods described herein may result in enhanced specificity of the effector functions of the immune cell for target cells (e.g., ADCC of cancer cells) and further reduce undesired side effects.

The Fc-containing polypeptide (e.g., antibodies) that bind the ACTR variants, such as therapeutic antibodies in afucosylated or mutated form as described herein, recognize a target such as a cell surface molecule, receptor, or carbohydrate on the surface of a target cell (e.g., a cancer cell). Immune cells that express an ACTR variant capable of binding such Fc-containing polypeptides (e.g., antibodies) recognize the target cell-bound antibodies and this receptor/antibody engagement stimulates the immune cell to perform effector functions such as release of cytotoxic granules or expression of cell-death-inducing molecules, leading to cell death of the target cell recognized by the Fc-containing molecules.

I. Chimeric Receptor Variants (ACTR Variants)

The chimeric receptor variants (ACTR variants) described herein comprise an extracellular domain, which can be a mutated extracellular ligand-binding domain of an Fc receptor (“mutated Fc binder”) or a scFv binding to a mutated Fc fragment, and a cytoplasmic signaling domain. The mutated Fc binder and the scFv fragment have a reduced binding activity to a wild-type Fc fragment as described herein but is capable of binding to a modified Fc domain, for example, an afucosylated Fc domain or a mutated Fc domain as described herein. Any of the Fc fragment described herein, including wild-type and modified, can be part of a polypeptide (an Fc-containing polypeptide), which may be an antibody. The wild-type Fc fragment may be part of an endogenous antibody molecule, part of a recombinantly produced antibody, or a recombinantly-produced Fc-containing polypeptide.

The ACTR variants have no or low binding activity to molecules having a wild-type Fc fragment, such as endogenous antibodies. Reducing or eliminating binding of an ACTR variant to the wild-type Fc fragment may result in a reduction of the activity of the ACTR variant induced by molecules (e.g., endogenous antibodies) containing a wild-type Fc fragment (e.g. the effector function of a host cell expressing the chimeric receptor variant, such as ADCC).

The chimeric receptor variants described herein may further comprise one or more additional domains, such as a transmembrane domain; zero, one or more co-stimulatory signaling domains; a hinge domain; or a combination thereof.

The chimeric receptor variants described herein are configured such that, when expressed on a host cell, the mutated Fc binder or the scFv fragment is located extracellularly for binding to a target molecule (e.g., an antibody or an Fc-containing polypeptide) and the co-stimulatory signaling domain is located in the cytoplasm for triggering activation and/or effector signaling. In some embodiments, a chimeric receptor variant construct as described herein comprises, from N-terminus to C-terminus, the mutated Fc binder and the cytoplasmic signaling domain. In some embodiments, a chimeric receptor variant construct as described herein comprises, from N-terminus to C-terminus, the mutated Fc binder, the transmembrane domain, and the cytoplasmic signaling domain. In some embodiments, a chimeric receptor variant construct as described herein comprises, from N-terminus to C-terminus, the mutated Fc binder, the co-stimulatory signaling domain, and the cytoplasmic signaling domain. In some embodiments, a chimeric receptor variant construct as described herein comprises, from N-terminus to C-terminus, the mutated Fc binder, the cytoplasmic signaling domain, and a co-stimulatory signaling domain. In some embodiments, a chimeric receptor variant construct as described herein comprises, from N-terminus to C-terminus, the mutated Fc binder, the transmembrane domain, and the cytoplasmic signaling domain. In some embodiments, a chimeric receptor variant construct as described herein comprises, from N-terminus to C-terminus, the mutated Fc binder, the transmembrane domain, the cytoplasmic signaling domain, and the co-stimulatory signaling domain. In some embodiments, a chimeric receptor variant construct as described herein comprises, from N-terminus to C-terminus, the mutated Fc binder, the transmembrane domain, the co-stimulatory signaling domain, and the cytoplasmic signaling domain. In some embodiments, the chimeric receptor does not comprise a co-stimulatory domain, but one or more separate polypeptides can be co-used with the ACTR variants to provide co-stimulatory signals in trans.

Any of the chimeric receptor variants described herein comprising a transmembrane domain may further comprise a hinge domain, which may be located at the C-terminus of the mutated Fc binder and the N-terminus of the transmembrane domain. Alternatively or in addition, the chimeric receptor variant constructs described herein may contain two or more co-stimulatory signaling domains, which may link to each other or be separated by the cytoplasmic signaling domain. The mutated Fc binder and cytoplasmic signaling domain, and optionally a transmembrane domain and/or co-stimulatory signaling domain, may be linked to each other directly, or via a peptide linker.

A. Extracellular Domains of the ACTR Variants

In some embodiments, the extracellular domain(s) of the ACTR variants described herein are mutated extracellular ligand-binding domains of Fc receptors (“mutated Fc binders”). The mutated Fc binder may contain a mutation at one or more residues relative to the wild-type counterpart that are involved in Fc/Fc receptor binding. Residues of an Fc fragment that is involved in Fc/Fc receptor binding include both residues that directly bind to the Fc receptor and residues that do not directly bind, but contribute to the binding of the Fc fragment to the Fc receptor (e.g., those that are essential to the formation of the binding pocket).

The extracellular ligand-binding domain of a suitable Fc receptors known in the art may be used for making the chimeric receptors described herein and may be subjected to mutation of one or more residues involved in the Fc/Fc receptor interaction. In general, an extracellular ligand-binding domain of an Fc receptor (“Fc binder”) is capable of binding to the Fc domain of an immunoglobulin (e.g., IgG, IgA, IgM, or IgE) of a suitable mammal (e.g., human, mouse, rat, goat, sheep, or monkey). Suitable Fc binders may be derived from naturally-occurring proteins such as mammalian Fc receptors and be subjected to mutation of one or more residues to reduce its binding activity to a wild-type Fc fragment.

As also used herein, an “Fc receptor” is a cell surface bound receptor that is expressed on the surface of many immune cells (including B cells, dendritic cells, natural killer (NK) cells, macrophage, neutrophils, mast cells, and eosinophils) and exhibits binding specificity to the Fc domain of an antibody. Fc receptors are typically comprised of at least 2 immunoglobulin (Ig)-like domains with binding specificity to an Fc (fragment crystallizable) portion of an antibody. In some instances, binding of an Fc receptor to an Fc portion of the antibody may trigger antibody dependent cell-mediated cytotoxicity (ADCC) effects.

In some embodiments, the mutated Fc binder is an extracellular ligand-binding domain of a mammalian Fc receptor that comprises a mutation at one or more residues relative to its wild-type counterpart (e.g., wild-type extracellular ligand-binding domain of a mammalian Fc receptor). As used herein, “a wild-type Fc receptor” refers to an Fc receptor that exists in nature, including polymorphism variants. In some embodiments, the mutations of the mutated Fc binder are made relative to the amino acid sequence of its wild-type counterpart. In some examples, the mutated Fc binder is derived from a wild-type CD16A for example, the wild-type CD16A set forth as SEQ ID NO: 18.

The Fc receptor used for constructing a chimeric receptor variant as described herein may be a naturally-occurring polymorphism variant (e.g., the CD16 V158 polymorphism variant having the amino acid sequence of SEQ ID NO: 18) and one or more mutations can be introduced at one or more residues involved in the interaction of the polymorphism variant with a naturally-occurring Fc domain of an immunoglobulin. Such mutations may reduce the binding activity of the Fc receptor for the naturally occurring Fc domain of an immunoglobulin.

TABLE 1
Exemplary Polymorphisms in Fc Receptors
(Kim, et al. J. Mol. Evol. (2001) 53(1): 1-9)

| Amino Acid | | | | | | | | | | |
|---|---|---|---|---|---|---|---|---|---|---|
| Number | 19 | 48 | 65 | 89 | 105 | 130 | 134 | 141 | 142 | 158 |
| FCR10 | R | S | D | I | D | G | F | Y | T | V |
| P08637 | R | S | D | I | D | G | F | Y | I | F |
| S76824 | R | S | D | I | D | G | F | Y | I | V |
| J04162 | R | N | D | V | D | D | F | H | I | V |
| M31936 | S | S | N | I | D | D | F | H | I | V |
| M24854 | S | S | N | I | E | D | S | H | I | V |
| X07934 | R | S | N | I | D | D | F | H | I | V |
| X14356 (FcγRII) | N | N | N | S | E | S | S | S | I | I |
| M31932 (FcγRI) | S | T | N | R | E | A | F | T | I | G |
| X06948 (FcαϵI) | R | S | E | S | Q | S | E | S | I | V |

Fc receptors are classified based on the isotype of the immunoglobulins to which it is able to bind. For example, Fc-gamma receptors (FcγR) generally bind to IgG antibodies, such as one or more subtype thereof (i.e., IgG1, IgG2, IgG3, IgG4); Fc-alpha receptors (FcaR) generally bind to IgA antibodies; and Fc-epsilon receptors (FcεR) generally bind to IgE antibodies. In some embodiments, the mutated Fc receptor is any one of an Fc-gamma receptor, an Fc-alpha receptor, or an Fc-epsilon receptor that comprises a mutation at one or more residues involved in interaction of the Fc receptor with an immunoglobulin. Examples of Fc-gamma receptors include, without limitation, CD64A, CD64B, CD64C, CD32A, CD32B, CD16A, and CD16B. In some embodiments, the Fc receptor than binds to IgG is FcRn. An example of an Fc-alpha receptor is FcαR1/CD89. Examples of Fc-epsilon receptors include, without limitation, FcεRI and FcεRII/CD23. The table below lists exemplary Fc receptors for use in constructing the chimeric receptors described herein and their binding activity to corresponding Fc domains:

TABLE 2
Exemplary Fc Receptors

| Receptor name | Principal antibody ligand | Affinity for ligand |
|---|---|---|
| FcγRI (CD64) | IgG1 and IgG3 | High (Kd~10⁻⁹ M) |
| FcγRIIA (CD32) | IgG | Low (Kd > 10⁻⁷ M) |
| FcγRIIB1 (CD32) | IgG | Low (Kd > 10⁻⁷ M) |
| FcγRIIB2 (CD32) | IgG | Low (Kd > 10⁻⁷ M) |
| FcγRIIIA (CD16a) | IgG | Low (Kd > 10⁻⁶ M) |
| Fc±RIIIB (CD16b) | IgG | Low (Kd > 10⁻⁶ M) |
| FcεRI | IgE | High (Kd~10⁻¹⁰ M) |
| FcεRII (CD23) | IgE | Low (Kd > 10⁻⁷ M) |
| FcαRI (CD89) | IgA | Low (Kd > 10⁻⁶ M) |
| Fcα/μR | IgA and IgM | High for IgM, Mid for IgA |
| FcRn | IgG | |

Selection of the ligand-binding domain of an Fc receptor for use in the chimeric receptors described herein will be apparent to one of skill in the art. For example, it may depend on factors such as the isotype of the antibody to which binding of the Fc receptor is desired or the binding affinity of the Fc receptor to its ligand, an Fc domain, for example of an antibody for use with the chimeric receptor.

Any of the mutated Fc binders described herein may have a suitable binding activity for a modified Fc domain, which may be afucosylated, mutated, or both. Similarly, any of the Fc binders described herein may be subjected to mutation to achieve a suitable (e.g., reduced or eliminated) binding activity to a wild-type Fc fragment. As used here, “binding activity” may encompass the activity induced by interaction of any of the chimeric receptors described herein with a target molecule, such as a desired activity (e.g., ADCC activity, gene expression, etc.). In some embodiments, the binding activity of the mutated Fc binder for a wild-type Fc fragment is about 1.5-fold, 2-fold, 3-fold, 4-fold, 5-fold, 6-fold, 7-fold, 8-fold, 9-fold, 10-fold, 20-fold, 30-fold, 40-fold, 50-fold, or at least 100-fold reduced as compared to binding activity of the Fc binder (in the absence of the one or more mutations) for the wild-type Fc fragment.

The binding activity of a chimeric receptor variant comprising a mutated Fc binder (e.g., an extracellular ligand-binding domain of an Fc receptor) or its wild-type counterpart for a wild-type Fc fragment can be determined by a variety of methods including physical binding assays, ADCC (cytotoxicity) assays, assessing expression of one or more genes, and/or activation of a signaling pathway in the cell expressing the chimeric receptor and/or a target cell.

In some embodiments, the Fc binders described herein may be subjected to mutation to achieve a suitable (e.g., reduced or eliminated) binding affinity to a wild-type Fc fragment. As used herein, “binding affinity” refers to the apparent association constant or K_{A}. The K_{A} is the reciprocal of the dissociation constant, K_{D}. The mutated extracellular ligand-binding domain of an Fc receptor domain of the chimeric receptors described herein may have a binding affinity K_{D} of at least 10⁻⁵, 10⁻⁶, 10⁻⁷, 10⁻⁸, 10⁻⁹, 10⁻¹⁰M or lower for a wild-type Fc fragment. In some embodiments, the mutated Fc binder has a reduced binding affinity for a specific wild-type Fc fragment, isotype, or subtype(s) thereof, as compared to the binding affinity of the mutated Fc binder to another Fc fragment, isotype of antibodies or subtypes thereof (e.g., an afucosylated antibody or an antibody that comprises one or more mutations relative to a wild-type antibody). In some embodiments, the binding affinity of the mutated Fc binder for a wild-type Fc fragment is about 1.5-fold, 2-fold, 3-fold, 4-fold, 5-fold, 6-fold, 7-fold, 8-fold, 9-fold, 10-fold, 20-fold, 30-fold, 40-fold, 50-fold, or at least 100-fold reduced as compared to binding affinity of the Fc binder (in the absence of the one or more mutations) for the wild-type Fc fragment.

The binding affinity of a chimeric receptor variant comprising a mutated Fc binder (e.g., an extracellular ligand-binding domain of an Fc receptor) or its wild-type counterpart for an Fc domain can be determined by a variety of methods including, without limitation, equilibrium dialysis, equilibrium binding, flow cytometery, gel filtration, ELISA, surface plasmon resonance, or spectroscopy.

In general, the terms “about” and “approximately” mean within an acceptable error range for the particular value as determined by one of ordinary skill in the art, which will depend in part on how the value is measured or determined, i.e., the limitations of the measurement system. For example, in regard to the binding activity of a chimeric receptor variant “about” can mean within an acceptable standard deviation, per the practice in the art. Alternatively, “about” can mean a range of up to ±30%, preferably up to ±20%, more preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Aspects of the present disclosure relate to mutation of one or more residues of an extracellular ligand-binding domain of an Fc receptor. In some embodiments, the one or more mutations result in a reduction in the binding activity of the extracellular ligand-binding domain of an Fc receptor to a protein containing a wild-type Fc fragment, such as an immunoglobulin or an Fc fusion protein. Residues of the extracellular ligand-binding domain of an Fc receptor that may be involved in interaction, direct or indirect, with a wild-type Fc fragment may be identified, for example, by assessing protein models of the interaction between an Fc receptor and an antibody or Fc domain. (See, for example, Lu et al. Proc. Natl. Acad. Sci. USA (2015) 112(3): 833-838; Radaev et al. J. Biol. Chem. (2011) 276 (19) 16469-16477; Mizushima et al. Genes Cells (2011) 11:1071-1080; Ahmed et al. J. Struct. Biol. (2016) 194:78-89; Ferrera et al. Proc. Natl. Acad. Sci. USA (2011) 108(31):12669-12674; and Sondermann et al. Nature (2000) 406 (6793):267-273). In some embodiments, one or more residues of the extracellular ligand-binding domain of an Fc receptor involved in direct interaction, or predicted to be in direct interaction, with a wild-type Fc fragment may be mutated, for example to reduce the direct interaction. In some embodiments, one or more residues of the extracellular ligand-binding domain of an Fc receptor involved in indirect interaction, or predicted to indirectly interact, with a wild-type Fc fragment may be mutated, for example to reduce interaction between the Fc receptor and the wild-type Fc fragment.

As would be appreciated by one of skill in the art, Fc receptors belonging to different superfamilies may share similar structure-functional correlation even if their primary amino acid sequences are different. Structural and sequence comparisons among Fc receptors were known in the art. See, e.g., Lu et al. J. Biol. Chem (2011) 286(47): 40608-40613. Mutation of an amino acid in a corresponding position in an Fc receptor belonging to different families or superfamilies may be made by comparing the secondary and/or tertiary structure of the Fc receptors to identify the relevant functional domains. In some embodiments, residues involved in the interaction between an Fc receptor and an Fc fragment may be identified based on sequence and/or structural alignment with other Fc receptors for which such residues are known, e.g., the FcγR reported in Lu et al., 2011. In some embodiments, the one or more mutations are of residues of the Fc receptor that are located or predicted to be located at the interface between the Fc receptor and an Fc region. In some embodiments, the one or more mutations are located in the Fc fragment binding pocket of the Fc receptor. In some embodiments, the one or more mutations are located in the D2 region of the extracellular ligand-binding domain of an Fc receptor. In some embodiments, the one or more mutations are located outside of the D2 region of the extracellular ligand-binding domain of an Fc receptor.

Without wishing to be bound by any particular theory, the one or more mutations may alter (enhance, reduce, or eliminate) glycosylation of the Fc receptor, which may thereby modulate (e.g., reduce or enhance) binding activity of the mutated Fc receptor to a wild-type Fc fragment. For example, the one or more mutations introduced into the extracellular domain of an Fc receptor may eliminate or reduce glycosylation of the Fc receptor, which in turn lead to reduced binding activity to a wild-type Fc fragment. In some embodiments, the immune cells expressing the ACTR are expanded under growth conditions that alter (enhance, reduce, or eliminate) glycosylation of the Fc receptor portion in the ACTR. In some embodiments, the immune cells expressing the ACTR are modified, for example, to express one or more glycosylation enzymes or glycosylation pathways, resulting in altered (enhanced, reduced, or eliminated) glycosylation of the Fc receptor portion in the ACTR.

As used herein, the term “mutation” may include a substitution mutation in which an amino acid is replaced with a different amino acid, or deletion mutation in which the amino acid at a given position is removed. The binding activity of a mutated Fc binder thus prepared to a wild-type Fc fragment and/or an antibody for use with the chimeric receptor can be verified by conventional methods and/or those described herein.

In some embodiments, the mutated extracellular ligand-binding domain of an Fc receptor comprises an amino acid sequence that is at least 90% (e.g., 91, 92, 93, 94, 95, 96, 97, 98, 99%) identical to the amino acid sequence of the extracellular ligand-binding domain of a wild-type Fc-gamma receptor, an Fc-alpha receptor, or an Fc-epsilon receptor. In some embodiments, the mutated extracellular ligand-binding domain of an Fc receptor comprises an amino acid sequence that is at least 90% (e.g., 91, 92, 93, 94, 95, 96, 97, 98, 99%) identical to the amino acid sequence of the extracellular ligand-binding domain of a wild-type Fc-gamma receptor, an Fc-alpha receptor, or an Fc-epsilon receptor, with regard to the residues involved in the interaction (direct or indirect) of the Fc receptor with a wild-type Fc fragment. The “percent identity” of two amino acid sequences can be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the disclosure. Where gaps exist between two sequences, Gapped BLAST can be utilized as described in Altschul et al. Nucleic Acids Res. 25(17):3389-3402, 1997. When utilizing BLAST and Gapped BLAST programs, the default parameters of the respective programs (e.g., XBLAST and NBLAST) can be used.

In some embodiments, the mutated extracellular ligand-binding domain of an Fc receptor may contain up to 10 (e.g., 9, 8, 7, 6, 5, 4, 3, 2, or 1) mutations (e.g., amino acid residue substitutions) as relative to the wild-type counterpart.

In some examples, the mutated ligand-binding domain of an Fc receptor can be derived from FcεRI, FcεRI/CD23, FcαRI/CD89, FcαμR, or FcRn. In some examples, the mutated extracellular ligand-binding domain of an Fc receptor can be derived from CD16A, CD16B, CD32A, CD32B, CD32C, CD64A, CD64B, CD64C, or a naturally-occurring polymorphism variant thereof as described herein (e.g., CD16A V158, CD16A F158, or any other example of a naturally-occurring polymorphism presented in Table 1). In some examples, the mutated ligand-binding domain of an Fc receptor is derived from CD16, such as CD16A, and comprises one or more mutations relative to the amino acid sequence of its wild-type counterpart.

The amino acid sequences of the CD16A V158 polymorphism variant are provided below. SEQ ID NO: 17 represents the amino acid sequence of the precursor receptor (including the signal sequence, which is underlined), and SEQ ID NO: 18 represents the amino acid sequence of the mature protein.

(CD16A V158, precursor protein)
SEQ ID NO: 17
MWQLLLPTALLLLVSAGMRTEDLPKAVVFLEPQWYRVLEKDSVTLKCQ
GAYSPEDNSTQWFHNESLISSQASSYFIDAATVDDSGEYRCQTNLSTL
SDPVQLEVHIGWLLLQAPRWVFKEEDPIHLRCHSWKNTALHKVTYLQN
GKGRKYFHHNSDFYIPKATLKDSGSYFCRGLVGSKNVSSETVNITITQ
GLAVSTISSFFPPGYQVSFCLVMVLLFAVDTGLYFSVKTNIRSSTRDW
KDHKFKWRKDPQDK
(CD16A V158, mature protein)
SEQ ID NO: 18
GMRTEDLPKAVVFLEPQWYRVLEKDSVTLKCQGAYSPEDNSTQWFHNE
SLISSQASSYFIDAATVDDSGEYRCQTNLSTLSDPVQLEVHIGWLLLQ
APRWVFKEEDPIHLRCHSWKNTALHKVTYLQNGKGRKYFHHNSDFYIP
KATLKDSGSYFCRGLVGSKNVSSETVNITITQGLAVSTISSFFPPGYQ
VSFCLVMVLLFAVDTGLYFSVKTNIRSSTRDWKDHKFKWRKDPQDK

In some embodiments, the mutated Fc binder is derived from CD16A. In some embodiments, the CD16A is a natural polymorphism, such as V158, described herein (SEQ ID NO:18) or F158. It is appreciated in the art that the V158 (or F158) polymorphism is referred to as such and corresponds to the amino acid at position 160 of the CD16A mature protein sequence. In some embodiments, the mutation is a substitution mutation of one or more amino acids corresponding to W92, K122, Y134, H136, V160, F160, G161, K163, and/or N164 in SEQ ID NO: 18 (in boldface above). As used herein, a position in any given sequence that corresponds to a position in a reference sequence refers to the counterpart position in the given sequence relative to the position in the reference sequence, even though the position may be numbered differently in the two sequences (e.g., due to a different numbering system or a different starting position used). Such a counterpart position can be readily identified by aligning the given sequence with the reference sequence following routine practice.

It would also be evident to one of skill in the art that the amino acids corresponding to W92, K122, Y134, H136, G161, K163, and/or N164 of the CD16A mature protein sequence may also be referred to in the art as W90, K120, Y132, H134, G159, K161, and/or N162. Selection of a suitable amino acid to substitute at a particular position will be evident to one of skill in the art and may be based on factors such as the properties of the side chain of the specific amino acid. In some embodiments, the one or more mutations is W92F, W92K, W92R, W92V, K122D, K122E, K122R, K122M, K122L, K122N, Y134W, Y134A, H136Y, H136W, H136F, V160W, V160K, V160D, V160Q, V160N, G161W, G161F, K163D, K163E, N164A and/or N164Q. It should be appreciated that the extracellular ligand-binding domain of an Fc receptor may further comprise mutation of any one or more additional residues that are not involved in the interaction of the Fc receptor and a wild-type Fc fragment. In some embodiments, the just-noted one or more mutations are the only mutations in a mutated Fc binder.

It would be evident to one of skill in the art that similar mutations may be made extracellular ligand-binding domains of different Fc receptors. For example, the corresponding amino acids of extracellular ligand-binding domains of a different Fc receptor may be identified by aligning the amino acid sequence of SEQ ID NO: 18 with the amino acid sequence of the extracellular ligand-binding domains of the different Fc receptor, using sequence alignment algorithms, such as CLUSTALW.

In other embodiments, the mutated Fc receptor can be derived from a non-CD16 receptor, such as CD32 or CD64, or others disclosed herein. The mutation(s) may occur in the residues that are involved in, or predicted to be involved in, direct or indirect interaction with the corresponding a Fc fragment. In some embodiments, the mutation(s) may occur in a domain of the Fc receptor (e.g., the D2 domain) that is involved in, or predicted to be involved in, direct or indirect interaction with an Fc fragment. Such functional domains are either known in the art (see Lu et al., 2011) or can be identified by performing sequence/structural alignment with Fc receptors having known sequence/structure correlation (e.g., FcγR disclosed in Lu et al., 2011). In some examples, the mutated Fc receptor may contain one or more mutations at positions corresponding to W92, K122, Y134, H136, V160, G161, K163, and/or N164 in SEQ ID NO: 18, which can be identified by performing structure/sequence alignment between SEQ ID NO:18 and the parent Fc receptor of the mutated Fc binder.

Specific examples of CD16A mutants include CD16A mutant V160Q, V160W, Y134A, K122L, and Y134A/N164Q (mutation positions correspond to positions 160, 134, 122, and 164 in SEQ ID NO: 18). In these specific examples, the called out amino acid substitutions are the only mutations relative to the wild-type CD16A counterpart (e.g., SEQ ID NO:18).

Also within the scope of the present disclosure are combinations of mutations in the extracellular ligand-binding domain of an Fc receptor. In some embodiments, the mutated extracellular ligand-binding domain of a Fc receptor comprises a mutation of at least 1, 2, 3, 4, 5, 6, 7, 8, 9, or at least 10 residues, relative to the wild-type counterpart, that are involved in interaction between the Fc receptor and a wild-type Fc fragment.

Alternatively, the extracellular domain of the chimeric receptor variant described herein may be a single chain antibody fragment that preferentially binds to a mutated Fc fragment as relative to a wild-type Fc fragment. A molecule is said to exhibit “preferential binding” if it reacts or associates more frequently, more rapidly, with greater duration and/or with greater affinity with a particular target antigen than it does with alternative targets. An antibody “preferentially binds” to a target antigen if it binds with greater affinity, avidity, more readily, and/or with greater duration than it binds to other substances. For example, an antibody that preferentially binds to a mutated Fc fragment is an antibody that binds this target antigen with greater affinity, avidity, more readily, and/or with greater duration than it binds to other antigens such as a wild-type Fc fragment. It is also understood by reading this definition that, for example, an antibody that preferentially binds to a first target antigen may or may not specifically or preferentially bind to a second target antigen. As such, “preferential binding” does not necessarily require (although it can include) exclusive binding.

The mutated Fc fragment may contain mutations at a suitable number of positions such that the mutated Fc fragment could induce antibodies having no or low cross reactivity to a wild-type Fc fragment. In some examples, the mutated Fc fragment shares at least 85% sequence identity (e.g., 90%, 95%, or 98%) with a wild-type Fc fragment. In some embodiments, the scFv fragment in the chimeric receptor variant does not bind a wild-type Fc fragment.

B. Cytoplasmic Signaling Domain

Any cytoplasmic signaling domain can be used to construct the chimeric receptors described herein. In general, a cytoplasmic signaling domain relays a signal, such as interaction of an extracellular ligand-binding domain with its ligand, to stimulate a cellular response, such inducing an effector function of the cell (e.g., ADCC).

In some embodiments, the cytoplasmic signaling domain comprises an immunoreceptor tyrosine-based inhibition motif (ITIM). In some embodiments, the cytoplasmic signaling domain comprises an immunoreceptor tyrosine-based activation motif (ITAM). An “ITIM” and an “ITAM” as used herein, are conserved protein motifs that are generally present in the tail portion of signaling molecules expressed in many immune cells.

The ITIM motif comprises the amino acid sequence S/I/V/LxYxxI/V/L. Upon stimulation of an ITIM, the motif becomes phosphorylated and reduces activation of molecules involved in cell signaling, thereby transducing an inhibitory signal. In some examples, the cytoplasmic domain comprising an ITIM is of a Killer-cell immunoglobulin-like receptor (KIR).

The ITAM motif may comprises two repeats of the amino acid sequence YxxL/I separated by 6-8 amino acids, wherein each x is independently any amino acid, producing the conserved motif YxxL/Ix₍₆₋₈₎YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways. In some examples, the cytoplasmic signaling domain comprising an ITAM is of CD3ζ or FcεR1γ. In other examples, the ITAM-containing cytoplasmic signaling domain is not derived from human CD3ζ. In yet other examples, the ITAM-containing cytoplasmic signaling domain is not derived from an Fc receptor, when the extracellular ligand-binding domain of the same chimeric receptor variant construct is derived from CD16A.

C. Transmembrane Domain

In some embodiments, the chimeric receptors described herein further comprise a transmembrane domain. Any transmembrane domain for use in the chimeric receptors can be in any form known in the art. As used herein, a “transmembrane domain” refers to any protein structure that is thermodynamically stable in a cell membrane, preferably a eukaryotic cell membrane. Transmembrane domains compatible for use in the chimeric receptors used herein may be obtained from a naturally-occurring protein. Alternatively, it can be a synthetic, non-naturally occurring protein segment, e.g., a hydrophobic protein segment that is thermodynamically stable in a cell membrane.

Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times).

Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain of the chimeric receptor variant described herein is derived from a Type I single-pass membrane protein. Single-pass membrane proteins include, but are not limited to, CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B. In some embodiments, the transmembrane domain is from a membrane protein selected from the following: CD8α, CD8β, 4-1BB/CD137, CD28, CD34, CD4, FcεRIγ, CD16, OX40/CD134, CD3ζ, CD3ε, CD3γ, CD3δ, TCRα, CD32, CD64, VEGFR2, FAS, and FGFR2B. In some examples, the transmembrane domain is of CD8α. In some examples, the transmembrane domain is of 4-1BB/CD137. In other examples, the transmembrane domain is of CD28 or CD34. In yet other examples, the transmembrane domain is not derived from human CD8α. In some embodiments, the transmembrane domain of the chimeric receptor variant is a single-pass alpha helix.

Transmembrane domains from multi-pass membrane proteins may also be compatible for use in the chimeric receptors described herein. Multi-pass membrane proteins may comprise a complex (at least 2, 3, 4, 5, 6, 7 or more) alpha helices or a beta sheet structure. Preferably, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side. Either one or multiple helix passes from a multi-pass membrane protein can be used for constructing the chimeric receptor variant described herein.

Transmembrane domains for use in the chimeric receptors described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 B1 and PCT Publication No. WO 2000/032776 A2, the relevant disclosures of which are incorporated by reference herein.

In some embodiments, the amino acid sequence of the transmembrane domain does not comprise cysteine residues. In some embodiments, the amino acid sequence of the transmembrane domain comprises one cysteine residue. In some embodiments, the amino acid sequence of the transmembrane domain comprises two cysteine residues. In some embodiments, the amino acid sequence of the transmembrane domain comprises more than two cysteine residues (e.g., 3, 4, 5 or more).

The transmembrane domain may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence.

The hydropathy, or hydrophobic or hydrophilic characteristics of a protein or protein segment, can be assessed by any method known in the art, for example the Kyte and Doolittle hydropathy analysis.

D. Co-Stimulatory Signaling Domains

Many immune cells require co-stimulation, in addition to stimulation of an antigen-specific signal, to promote cell proliferation, differentiation and survival, as well as to activate effector functions of the cell. In some embodiments, the chimeric receptors described herein comprise a co-stimulatory signaling domain. In some embodiments, the chimeric receptors described herein comprise a more than one co-stimulatory signaling domain. The term “co-stimulatory signaling domain,” as used herein, refers to at least a portion of a protein that mediates signal transduction within a cell to induce an immune response, such as an effector function. The co-stimulatory signaling domain of the chimeric receptor variant described herein can be a cytoplasmic signaling domain from a co-stimulatory protein, which transduces a signal and modulates responses mediated by immune cells, such as T cells, NK cells, macrophages, neutrophils, or eosinophils.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the chimeric receptors described herein. The type(s) of co-stimulatory signaling domain is selected based on factors such as the type of the immune cells in which the chimeric receptors would be expressed (e.g., T cells, NK cells, macrophages, neutrophils, or eosinophils) and the desired immune effector function (e.g., ADCC effect). Examples of co-stimulatory signaling domains for use in the chimeric receptors can be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACl/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. In some embodiments, the co-stimulatory signaling domain is of 4-1BB, CD28, OX40, ICOS, CD27, GITR, HVEM, TIM1, LFA1(CD11a) or CD2, or any variant thereof. In other embodiments, the co-stimulatory signaling domain is not derived from 4-1BB.

Also within the scope of the present disclosure are variants of any of the co-stimulatory signaling domains described herein, such that the co-stimulatory signaling domain is capable of modulating the immune response of the immune cell. In some embodiments, the co-stimulatory signaling domains comprises up to 10 amino acid residue variations (e.g., 1, 2, 3, 4, 5, or 8) as compared to a wild-type counterpart. Such co-stimulatory signaling domains comprising one or more amino acid variations may be referred to as variants.

Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation. For example, mutation of residues 186 and 187 of the native CD28 amino acid sequence may result in an increase in co-stimulatory activity and induction of immune responses by the co-stimulatory domain of the chimeric receptor. In some embodiments, the mutations are substitution of a lysine at each of positions 186 and 187 with a glycine residue of the CD28 co-stimulatory domain, referred to as a CD28_{LL→GG} variant. Additional mutations that can be made in co-stimulatory signaling domains that may enhance or reduce co-stimulatory activity of the domain will be evident to one of ordinary skill in the art. In some embodiments, the co-stimulatory signaling domain is of 4-1BB, CD28, OX40, or CD28_{LL→GG} variant.

In some embodiments, the chimeric receptors may comprise more than one co-stimulatory signaling domain (e.g., 2, 3 or more). In some embodiments, the chimeric receptor variant comprises two or more of the same co-stimulatory signaling domains, for example, two copies of the co-stimulatory signaling domain of CD28. In some embodiments, the chimeric receptor variant comprises two or more co-stimulatory signaling domains from different co-stimulatory proteins, such as any two or more co-stimulatory proteins described herein. Selection of the type(s) of co-stimulatory signaling domains may be based on factors such as the type of host cells to be used with the chimeric receptors (e.g., immune cells such as T cells, NK cells, macrophages, neutrophils, or eosinophils) and the desired immune effector function. In some embodiments, the chimeric receptor variant comprises two co-stimulatory signaling domains. In some embodiments, the two co-stimulatory signaling domains are CD28 and 4-1BB. In some embodiments, the two co-stimulatory signaling domains are CD28_{LL→GG} variant and 4-1BB.

Any of the co-stimulatory domains, or a combination thereof, may be part of the ACTR variants described herein. ACTR variants that contain a co-stimulatory signaling domain may be co-used (co-introduced into a host cell) with a separate polypeptide, which can be a co-stimulatory factor or comprises the co-stimulatory domain thereof. The separate polypeptide may comprise the same co-stimulatory domain as the ACTR variant, or a different co-stimulatory domain. ACTR variants that contain a co-stimulatory signaling domain may also be co-used with a separate polypeptide comprising a ligand of a co-stimulatory factor, which can be the same as or different from that used in the ACTR variant. See, e.g., Zhao, et al. Cancer Cell (2015) 28:415-428.

Alternatively, ACTR variants that do not contain a co-stimulatory domain can be co-used (co-introduced into a host cell) with a separate polypeptide, which can be a co-stimulatory factor or comprises the co-stimulatory domain thereof. ACTR variants that do not contain a co-stimulatory signaling domain may also be co-used with a separate polypeptide comprising a ligand of a co-stimulatory factor.

E. Hinge Domain

In some embodiments, the chimeric receptors described herein further comprise a hinge domain that is located between the extracellular ligand-binding domain and the transmembrane domain. A hinge domain is an amino acid segment that is generally found between two domains of a protein and may allow for flexibility of the protein and movement of one or both of the domains relative to one another. Any amino acid sequence that provides such flexibility and movement of the extracellular ligand-binding domain of an Fc receptor relative to the transmembrane domain of the chimeric receptor variant can be used.

The hinge domain may contain about 10-200 amino acids, e.g., 15-150 amino acids, 20-100 amino acids, or 30-60 amino acids. In some embodiments, the hinge domain may be of about 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, or 200 amino acids in length.

In some embodiments, the hinge domain is a hinge domain of a naturally-occurring protein. Hinge domains of any protein known in the art to comprise a hinge domain are compatible for use in the chimeric receptors described herein. In some embodiments, the hinge domain is at least a portion of a hinge domain of a naturally-occurring protein and confers flexibility to the chimeric receptor. In some embodiments, the hinge domain is of CD8α. In some embodiments, the hinge domain is a portion of the hinge domain of CD8α, e.g., a fragment containing at least 15 (e.g., 20, 25, 30, 35, or 40) consecutive amino acids of the hinge domain of CD8α.

Hinge domains of antibodies, such as an IgG, IgA, IgM, IgE, or IgD antibody, are also compatible for use in the chimeric receptors described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge domains for the chimeric receptors described herein. In some embodiments, the hinge domain between the C-terminus of the extracellular ligand-binding domain of an Fc receptor and the N-terminus of the transmembrane domain is a peptide linker, such as a (GlyₓSer)ₙ linker, wherein x and n, independently can be an integer between 3 and 12, including 3, 4, 5, 6, 7, 8, 9, 10, 11, 12, or more. In some embodiments, the hinge domain is (Gly₄Ser)ₙ (SEQ ID NO: 79), wherein n can be an integer between 3 and 60, including 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more. In some embodiments, the hinge domain is (Gly₄Ser)₃ (SEQ ID NO: 19). In some embodiments, the hinge domain is (Gly₄Ser)₆ (SEQ ID NO: 20). In some embodiments, the hinge domain is (Gly₄Ser)₉ (SEQ ID NO: 21). In some embodiments, the hinge domain is (Gly₄Ser)₁₂ (SEQ ID NO: 22). In some embodiments, the hinge domain is (Gly₄Ser)ᵢₛ (SEQ ID NO: 23). In some embodiments, the hinge domain is (Gly₄Ser)₃₀ (SEQ ID NO: 24). In some embodiments, the hinge domain is (Gly₄Ser)₄₅ (SEQ ID NO: 25). In some embodiments, the hinge domain is (Gly₄Ser)₆₀ (SEQ ID NO: 26).

In other embodiments, the hinge domain is an extended recombinant polypeptide (XTEN), which is an unstructured polypeptide consisting of hydrophilic residues of varying lengths (e.g., 10-200 amino acid residues, 20-150 amino acid residues, 30-100 amino acid residues, or 40-80 amino acid residues). Amino acid sequences of XTEN peptides will be evident to one of skill in the art and can be found, for example, in U.S. Pat. No. 8,673,860, which is herein incorporated by reference. In some embodiments, the hinge domain is an XTEN peptide and comprises 60 amino acids. In some embodiments, the hinge domain is an XTEN peptide and comprises 30 amino acids. In some embodiments, the hinge domain is an XTEN peptide and comprises 45 amino acids. In some embodiments, the hinge domain is an XTEN peptide and comprises 15 amino acids.

F. Signal Peptide

In some embodiments, the chimeric receptor variant also comprises a signal peptide (also known as a signal sequence) at the N-terminus of the polypeptide. In general, signal sequences are peptide sequences that target a polypeptide to the desired site in a cell. In some embodiments, the signal sequence targets the chimeric receptor variant to the secretory pathway of the cell and will allow for integration and anchoring of the chimeric receptor variant into the lipid bilayer. Signal sequences including signal sequences of naturally-occurring proteins or synthetic, non-naturally-occurring signal sequences, that are compatible for use in the chimeric receptors described herein will be evident to one of skill in the art. In some embodiments, the signal sequence from CD8α. In some embodiments, the signal sequence is from CD28. In other embodiments, the signal sequence is from the murine kappa chain. In yet other embodiments, the signal sequence is from CD16. An example signal sequence is provided by amino acid residues 1-16 of SEQ ID NO: 17.

Amino acid sequences of the example ACTR variants are provided below.

ACTR variant 1
(SEQ ID NO: 1)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 26
(SEQ ID NO: 2)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHDV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 89
(SEQ ID NO: 3)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHEV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 90
(SEQ ID NO: 4)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHRV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 92
(SEQ ID NO: 5)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHMV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 93
(SEQ ID NO: 6)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHLV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 94
(SEQ ID NO: 7)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLWGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 95
(SEQ ID NO: 8)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLDGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 96
(SEQ ID NO: 9)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLKGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 97
(SEQ ID NO: 10)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVFSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 98
(SEQ ID NO: 11)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVWSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 99
(SEQ ID NO: 12)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFYHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 100
(SEQ ID NO: 13)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFFHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 101
(SEQ ID NO: 14)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFWHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 102
(SEQ ID NO: 15)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKWFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 103
(SEQ ID NO: 16)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKAFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 104
(SEQ ID NO: 31)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKQVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 105
(SEQ ID NO: 32)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKAVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 106
(SEQ ID NO: 33)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHDV
TYLQNGKGRKYFHHNSDFYIPKATLKDSGSYFCRGLVGSDNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 107
(SEQ ID NO: 34)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHDV
TYLQNGKGRKYFHHNSDFYIPKATLKDSGSYFCRGLVGSDQVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 108
(SEQ ID NO: 35)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHEV
TYLQNGKGRKYFHHNSDFYIPKATLKDSGSYFCRGLVGSENVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 109
(SEQ ID NO: 36)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHEV
TYLQNGKGRKYFHHNSDFYIPKATLKDSGSYFCRGLVGSEQVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 110
(SEQ ID NO: 37)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSDNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 111
(SEQ ID NO: 38)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSENVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 112
(SEQ ID NO: 39)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSDQVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 113
(SEQ ID NO: 40)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSEQVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 114
(SEQ ID NO: 41)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKAFHHNSDFYIPKATLKDSGSYFCRGLVGSKQVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 115
(SEQ ID NO: 42)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHDV
TYLQNGKGRKYFHHNSDFYIPKATLKDSGSYFCRGLVGSKQVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 116
(SEQ ID NO: 43)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHEV
TYLQNGKGRKYFHHNSDFYIPKATLKDSGSYFCRGLVGSKQVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 117
(SEQ ID NO: 44)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHDV
TYLQNGKGRKAFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 118
(SEQ ID NO: 45)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHNV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 119
(SEQ ID NO: 46)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHNV
TYLQNGKGRKYFHHNSDFYIPKATLKDSGSYFCRGLVGSKQVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 120
(SEQ ID NO: 47)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKAFHHNSDFYIPKATLKDSGSYFCRGLWGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 121
(SEQ ID NO: 48)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHDV
TYLQNGKGRKAFHHNSDFYIPKATLKDSGSYFCRGLVGSKQVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 122
(SEQ ID NO: 49)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHEV
TYLQNGKGRKAFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 123
(SEQ ID NO: 50)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHEV
TYLQNGKGRKAFHHNSDFYIPKATLKDSGSYFCRGLVGSKQVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 124
(SEQ ID NO: 51)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHDV
TYLQNGKGRKAFHHNSDFYIPKATLKDSGSYFCRGLWGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 125
(SEQ ID NO: 52)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHDV
TYLQNGKGRKAFHHNSDFYIPKATLKDSGSYFCRGLWGSKQVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 126
(SEQ ID NO: 53)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLNGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 127
(SEQ ID NO: 54)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLQGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 128
(SEQ ID NO: 55)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLWGSKQVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 129
(SEQ ID NO: 56)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHMV
TYLQNGKGRKYFHHNSDFYIPKATLKDSGSYFCRGLVGSKQVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 130
(SEQ ID NO: 57)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHLV
TYLQNGKGRKYFHHNSDFYIPKATLKDSGSYFCRGLVGSKQVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 131
(SEQ ID NO: 58)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKWFHHNSDFYIPKATLKDSGSYFCRGLVGSKQVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 132
(SEQ ID NO: 59)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGKLLLQAPRWVFKEEDPIHLRCHSWKNTALHKV
TYLQNGKGRKYFHHNSDFYIPKATLKDSGSYFCRGLDGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 133
(SEQ ID NO: 60)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGRLLLQAPRWVFKEEDPIHLRCHSWKNTALHKV
TYLQNGKGRKYFHHNSDFYIPKATLKDSGSYFCRGLDGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 134
(SEQ ID NO: 61)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGFLLLQAPRWVFKEEDPIHLRCHSWKNTALHKV
TYLQNGKGRKYFHHNSDFYIPKATLKDSGSYFCRGLDGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 135
(SEQ ID NO: 62)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGVLLLQAPRWVFKEEDPIHLRCHSWKNTALHKV
TYLQNGKGRKYFHHNSDFYIPKATLKDSGSYFCRGLDGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 136
(SEQ ID NO: 63)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGKLLLQAPRWVFKEEDPIHLRCHSWKNTALHKV
TYLQNGKGRKYFHHNSDFYIPKATLKDSGSYFCRGLDGSKQVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 137
(SEQ ID NO: 64)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGRLLLQAPRWVFKEEDPIHLRCHSWKNTALHKV
TYLQNGKGRKYFHHNSDFYIPKATLKDSGSYFCRGLDGSKQVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 138
(SEQ ID NO: 65)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGFLLLQAPRWVFKEEDPIHLRCHSWKNTALHKV
TYLQNGKGRKYFHHNSDFYIPKATLKDSGSYFCRGLDGSKQVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 139
(SEQ ID NO: 66)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGVLLLQAPRWVFKEEDPIHLRCHSWKNTALHKV
TYLQNGKGRKYFHHNSDFYIPKATLKDSGSYFCRGLDGSKQVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 140
(SEQ ID NO: 67)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHMV
TYLQNGKGRKYFHHNSDFYIPKATLKDSGSYFCRGLWGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 141
(SEQ ID NO: 68)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHMV
TYLQNGKGRKAFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
ACTR variant 142
(SEQ ID NO: 69)
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHMV
TYLQNGKGRKAFHHNSDFYIPKATLKDSGSYFCRGLWGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR

Production

Any of the chimeric receptors described herein can be prepared by a routine method, such as recombinant technology. Methods for preparing the chimeric receptors herein involve generation of a nucleic acid that encodes a polypeptide comprising each of the domains of the chimeric receptors, including the mutated extracellular ligand-binding domain of an Fc receptor and the cytoplasmic signaling domain. In some embodiments, the nucleic acid also encodes any one or more of a transmembrane domain, a co-stimulatory signaling domain, and a hinge domain between the mutated extracellular ligand-binding domain of an Fc receptor and the transmembrane domain. The nucleic acid encoding the chimeric receptor variant may also encode a signal sequence. In some embodiments, the nucleic acid sequence encodes any one of the exemplary chimeric receptors provided by SEQ ID NO: 1-16 and 31-69.

Sequences of each of the components of the chimeric receptors may be obtained via routine technology, e.g., PCR amplification from any one of a variety of sources known in the art. In some embodiments, sequences of one or more of the components of the chimeric receptors are obtained from a human cell. Alternatively, the sequences of one or more components of the chimeric receptors can be synthesized. Sequences of each of the components (e.g., domains) can be joined directly or indirectly (e.g., using a nucleic acid sequence encoding a peptide linker) to form a nucleic acid sequence encoding the chimeric receptor, using methods such as PCR amplification or ligation. Mutation of one or more residues, for example one or more residues within the extracellular ligand-binding domain that are involved in interaction of the Fc receptor with an antibody, may be made in the nucleic acid sequence encoding said domain prior to or after joining the sequences of each of the components. Alternatively, the nucleic acid encoding the chimeric receptor variant may be synthesized. In some embodiments, the nucleic acid is DNA. In other embodiments, the nucleic acid is RNA.

Any of the chimeric receptor variant proteins, nucleic acid encoding such, and expression vectors carrying such nucleic acid can be mixed with a pharmaceutically acceptable carrier to form a pharmaceutical composition, which is also within the scope of the present disclosure.

II. Immune Cells Expressing Chimeric Receptors

Host cells expressing the chimeric receptor variants (ACTR variants) described herein provide a specific population of cells that can recognize target cells (e.g., cancer cells) bound by non-naturally occurring antibodies or Fc-fusion proteins. The host cells expressing the chimeric receptor variants described herein do not recognize or have reduced activity towards wild-type Fc fragments, thereby reducing or preventing recognition of non-target cells (e.g., cells bound by antibodies or proteins containing wild-type Fc fragments).

Engagement of the mutated extracellular ligand-binding domain of a chimeric receptor variant construct expressed on such host cells (e.g., immune cells) with the Fc portion of an antibody or an Fc-fusion protein transmits an activation signal to the cytoplasmic signaling domain, and optionally the one or more co-stimulatory domains, of the chimeric receptor variant construct, which in turn activates cell proliferation and/or effector functions of the host cell, such as ADCC effects triggered by the host cells. In some embodiments, the chimeric receptors also comprise one or more co-stimulatory signaling domain(s). Such configuration may allow for robust activation of multiple signaling pathways within the cell. The mutated extracellular ligand-binding domain of the chimeric receptors described herein reduce or prevent binding of the host cell expressing the chimeric receptor variant to a wild-type Fc fragment, thereby reducing or preventing competitive binding of wild-type Fc fragments and enhancing the efficacy of the antibody-immunotherapy. The reduced or eliminated binding of host cells expressing the chimeric receptors to wild-type Fc fragments may also reduce or prevent autoimmune reactions in which the effector functions (e.g., ADCC) of the host cell are activated subsequent to binding of a wild-type Fc fragment to a non-target cell.

In some embodiments, the host cells are immune cells, such as T cells, NK cells, macrophages, neutrophils, eosinophils, or any combination thereof. In some embodiments, the immune cells are T cells. In some embodiments, the immune cells are NK cells. In other embodiments, the immune cells can be established cell lines, for example, NK-92 cells.

The population of immune cells can be obtained from any source, such as peripheral blood mononuclear cells (PBMCs), bone marrow, tissues such as spleen, lymph node, thymus, or tumor tissue. A source suitable for obtaining the type of host cells desired would be evident to one of skill in the art. In some embodiments, the population of immune cells is derived from PBMCs. In some embodiments, the population of immune cells is derived from a human cancer patient, such as from the bone marrow or from a tumor in a human cancer patient. In some embodiments, the population of immune cells is derived from a healthy donor. The type of host cells desired (e.g., immune cells such as T cells, NK cells, macrophages, neutrophils, eosinophils, or any combination thereof) may be expanded within the population of cells obtained by co-incubating the cells with stimulatory molecules, for example, anti-CD3 and anti-CD28 antibodies may be used for expansion of T cells.

To construct the immune cells that express any of the chimeric receptor variant constructs described herein, expression vectors for stable or transient expression of the chimeric receptor variant construct may be constructed via conventional methods as described herein and introduced into immune host cells. For example, nucleic acids encoding the chimeric receptor variants may be cloned into a suitable expression vector, such as a viral vector in operable linkage to a suitable promoter. The nucleic acids and the vector may be contacted, under suitable conditions, with a restriction enzyme to create complementary ends on each molecule that can pair with each other and be joined with a ligase. Alternatively, synthetic nucleic acid linkers can be ligated to the termini of the nucleic acid encoding the chimeric receptors. The synthetic linkers may contain nucleic acid sequences that correspond to a particular restriction site in the vector. The selection of expression vectors/plasmids/viral vectors would depend on the type of host cells for expression of the chimeric receptors, but should be suitable for integration and replication in eukaryotic cells.

A variety of promoters can be used for expression of the chimeric receptors described herein, including, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters for expression of the chimeric receptors include any constitutively active promoter in an immune cell. Alternatively, any regulatable promoter may be used, such that its expression can be modulated within an immune cell.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; 5′- and 3′-untranslated regions for mRNA stability and translation efficiency from highly-expressed genes like α-globin or (3-globin; SV40 polyoma origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the chimeric receptor. See section VI below. Suitable vectors and methods for producing vectors containing transgenes are well known and available in the art. Examples of the preparation of vectors for expression of chimeric receptors can be found, for example, in US2014/0106449, herein incorporated by reference in its entirety.

In some embodiments, the chimeric receptor variant construct or the nucleic acid encoding said chimeric receptor variant is a DNA molecule. In some embodiments, the chimeric receptor variant construct or the nucleic acid encoding said chimeric receptor variant is a transposon. In some embodiments, the chimeric receptor variant construct or the nucleic acid encoding said chimeric receptor variant is a plasmid. In some embodiments, chimeric receptor variant construct or the nucleic acid encoding said chimeric receptor variant is a DNA plasmid may be electroporated to immune cells (see, e.g., Till, et al. Blood (2012) 119(17): 3940-3950). In some embodiments, the nucleic acid encoding the chimeric receptor variant is an RNA molecule, which may be electroporated to immune cells.

Any of the vectors comprising a nucleic acid sequence that encodes a chimeric receptor variant construct described herein is also within the scope of the present disclosure. Such a vector may be delivered into host cells such as host immune cells by a suitable method. Methods of delivering vectors to immune cells are well known in the art and may include DNA, RNA, or transposon electroporation; transfection reagents such as liposomes or nanoparticles to deliver DNA, RNA, or transposons; delivery of DNA, RNA, transposons, or protein by mechanical deformation (see, e.g., Sharei et al. Proc. Natl. Acad. Sci. USA (2013)110(6): 2082-2087); or viral transduction. In some embodiments, the vectors for expression of the chimeric receptors are delivered to host cells by viral transduction. Exemplary viral methods for delivery include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors, and adeno-associated virus (AAV) vectors (see, e.g., PCT Publication Nos. WO 94/12649, WO 93/03769; WO 93/19191; WO 94/28938; WO 95/11984 and WO 95/00655). In some embodiments, the vectors for expression of the chimeric receptors are retroviruses. In some embodiments, the vectors for expression of the chimeric receptors are lentiviruses. In some embodiments, the vectors for expression of the chimeric receptors are gamma-retroviruses. In some embodiments, the vectors for expression of the chimeric receptors are adeno-associated viruses.

In examples in which the vectors encoding chimeric receptor variants are introduced to the host cells using a viral vector, viral particles that are capable of infecting the immune cells and carry the vector may be produced by any method known in the art and can be found, for example in PCT Application No. WO 1991/002805A2, WO 1998/009271 A1, and U.S. Pat. No. 6,194,191. The viral particles are harvested from the cell culture supernatant and may be isolated and/or purified prior to contacting the viral particles with the immune cells.

Following introduction into the host cells a vector encoding any of the chimeric receptor variants provided herein, the cells are cultured under conditions that allow for expression of the chimeric receptor. In examples in which the nucleic acid encoding the chimeric receptor variant is regulated by a regulatable promoter, the host cells are cultured in conditions wherein the regulatable promoter is activated. In some embodiments, the promoter is an inducible promoter and the immune cells are cultured in the presence of the inducing molecule or in conditions in which the inducing molecule is produced. Determining whether the chimeric receptor variant is expressed will be evident to one of skill in the art and may be assessed by any known method, for example, detection of the chimeric receptor variant-encoding mRNA by quantitative reverse transcriptase PCR (qRT-PCR) or detection of the chimeric receptor variant protein by methods including Western blotting, fluorescence microscopy, and flow cytometry. Alternatively, expression of the chimeric receptor variant may take place in vivo after the immune cells are administered to a subject.

Alternatively, expression of a chimeric receptor variant construct in any of the immune cells disclosed herein can be achieved by introducing RNA molecules encoding the chimeric receptor variant constructs. Such RNA molecules can be prepared by in vitro transcription or by chemical synthesis. The RNA molecules can then introduced into suitable host cells such as immune cells (e.g., T cells, NK cells, macrophages, neutrophils, eosinophils, or any combination thereof) by, e.g., electroporation, transfection reagents, viral transduction or mechanical deformation of cells. For example, RNA molecules can be synthesized and introduced into host immune cells following the methods described in Rabinovich et al., Human Gene Therapy, 17:1027-1035 and WO WO2013/040557.

Methods for preparing host cells expressing any of the chimeric receptor variants described herein may also comprise activating the host cells ex vivo or in vivo. Activating a host cell means stimulating a host cell into an activate state in which the cell may be able to perform effector functions (e.g., ADCC). Methods of activating a host cell will depend on the type of host cell used for expression of the chimeric receptors. For example, T cells may be activated ex vivo in the presence of one or more molecule such as an anti-CD3 antibody, an anti-CD28 antibody, IL-2, IL-17, IL-15, or phytohemoagglutinin. In other examples, NK cells may be activated ex vivo in the presence of one or molecules such as a 4-1BB ligand, an anti-4-1BB antibody, IL-15, an anti-IL-15 receptor antibody, IL-2, IL12, IL-21, and K562 cells. In some embodiments, the host cells expressing any of the chimeric receptor variants described herein are activated ex vivo prior to administration to a subject. Determining whether a host cell is activated will be evident to one of skill in the art and may include assessing expression of one or more cell surface markers associated with cell activation, expression or secretion of cytokines, and cell morphology.

The methods of preparing host cells expressing any of the chimeric receptor variants described herein may comprise expanding the host cells ex vivo. Expanding host cells may involve any method that results in an increase in the number of cells expressing chimeric receptors, for example, allowing the host cells to proliferate or stimulating the host cells to proliferate. Methods for stimulating expansion of host cells will depend on the type of host cell used for expression of the chimeric receptors and will be evident to one of skill in the art. In some embodiments, the host cells expressing any of the chimeric receptor variants described herein are expanded ex vivo prior to administration to a subject.

In some embodiments, the host cells expressing the chimeric receptor variants are expanded and activated ex vivo prior to administration of the cells to the subject.

IV. Application of Immune Cells Expressing ACTR Variants in Immunotherapy

Host cells (e.g., immune cells) expressing the chimeric receptor variants (the encoding nucleic acids or vectors comprising such) described herein are useful enhancing the efficacy of an antibody-based immunotherapy and reducing autoimmune responses in a subject, particularly immunotherapies involving the use of non-naturally occurring antibodies that are capable of binding to the ACTR variants expressed on the immune cells. As used herein, the term “subject” refers to any mammal, such as a human, monkey, mouse, rabbit, or domestic mammal. In some embodiments, the subject is a human. In some embodiments, the subject is a human cancer patient. In some embodiments, the subject has been treated or is being treated with any of the non-naturally occurring antibodies capable of being bound by the mutated extracellular ligand-binding domain of an Fc receptor.

The immune cells can be mixed with a pharmaceutically acceptable carrier to form a pharmaceutical composition, which is also within the scope of the present disclosure. In some embodiments, the pharmaceutical composition also includes a non-naturally occurring antibody.

To perform the methods described herein, a therapeutically effective amount of the immune cells expressing any of the chimeric receptor variant constructs described herein and a therapeutically effective amount of a non-naturally occurring antibody that binds the chimeric receptor variant can be co-administered to a subject in need of the treatment. As used herein the term “therapeutically effective amount” refers to that quantity of a compound, cell population (e.g., immune cells expressing the chimeric receptors described herein), nucleic acid, antibody, or pharmaceutical composition (e.g., a composition comprising immune cells such as T lymphocytes and/or NK cells) comprising a chimeric receptor variant of the disclosure, and optionally further comprising a non-naturally occurring antibody that binds the chimeric receptor variant or another anti-tumor molecule comprising the Fc portion (e.g., a fusion protein constituted by a ligand (e.g., cytokine, immune cell receptor) binding a tumor surface receptor combined with the Fc-portion of an immunoglobulin or Fc-containing DNA or RNA)) that is sufficient to result in a desired activity upon administration to a subject in need thereof. Within the context of the present disclosure, the term “therapeutically effective amount” refers to that quantity of a compound, cell population, nucleic acid, or pharmaceutical composition that is sufficient to delay the manifestation, arrest the progression, relieve or alleviate at least one symptom of a disorder treated by the methods of the present disclosure. Note that when a combination of active ingredients is administered the effective amount of the combination may or may not include amounts of each ingredient that would have been effective if administered individually.

The immune cells expressing the chimeric receptor variants described herein may be autologous to the subject, i.e., the immune cells are obtained from the subject in need of the treatment, genetically engineered for expression of the chimeric receptor variant constructs, and then administered to the same subject. Administration of autologous cells to a subject may result in reduced rejection of the host cells as compared to administration of non-autologous cells. Alternatively, the host cells are allogeneic cells, i.e., the cells are obtained from a first subject, genetically engineered for expression of the chimeric receptor variant construct, and administered to a second subject that is different from the first subject but of the same species. For example, allogeneic immune cells may be derived from a human donor and administered to a human recipient who is different from the donor.

The T lymphocyte may be an allogeneic T lymphocyte. Such T lymphocytes may be from donors with partially matched HLA subtypes or with epigenetic profiles with reduced chance for inducing graft versus host disease. Alternatively, virally-selected T lymphocytes may be used. In some examples, the allogeneic T cells can be engineered to reduce the graft versus host effects. For example, the expression of the endogenous T cell receptor can be inhibited or eliminated. Alternatively or in addition, expression of one or more components of the Major Histocompatibility Complex (MHC) Class I and/or Class II complex (e.g., β-2-microglobulin) can be reduced or eliminated. In other examples, a natural killer cell inhibitory receptor can be expressed on the T lymphocyte.

In general, antibody-based immunotherapy is used to treat, alleviate, or reduce the symptoms of any disease or disorder for which the immunotherapy is considered useful in a subject. In the context of the present disclosure insofar as it relates to any of the disease conditions recited herein, the terms “treat,” “treatment,” and the like mean to relieve or alleviate at least one symptom associated with such condition, or to slow or reverse the progression of such condition. Within the meaning of the present disclosure, the term “treat” also denotes to arrest, delay the onset (i.e., the period prior to clinical manifestation of a disease) and/or reduce the risk of developing or worsening a disease. For example, in connection with cancer the term “treat” may mean eliminate or reduce a patient's tumor burden, or prevent, delay or inhibit metastasis, etc.

As described herein, the chimeric receptor variants comprise a mutated extracellular ligand-binding domain of an Fc receptor or an scFv that binds a modified Fc fragment as described herein. In some embodiments, host cells expressing the chimeric receptors described herein are administered in the presence of or in combination with a non-naturally occurring antibody (e.g. a therapeutic antibody that has been modified and/or mutated, or an afucosylated therapeutic antibody, or a therapeutic antibody that has been modified and/or mutated and is afucosylated). In immunotherapy, the non-naturally-occurring antibody (e.g. a modified and/or mutated therapeutic antibody) may bind to a cell surface antigen that is differentially expressed on cancer cells (i.e., not expressed on non-cancer cells or expressed at a lower level on non-cancer cells). Examples of antigens or target molecules that are bound by therapeutic antibodies and indicate that the cell expressing the antigen or target molecule should be subjected to ADCC include, without limitation, CD17/L1-CAM, CD19, CD20, CD22, CD30, CD33, CD37, CD52, CD56, CD70, CD79b, CD138, CEA, DS6, EGFR, EGFRvIII, ENPP3, FR, GD2, GPNMB, HER2, IL-13Rα2, Mesothelin, MUC1, MUC16, Nectin-4, PSMA, and SCL44A4. One advantage of the chimeric receptor variants described herein is that due to the reduced binding affinity of the chimeric receptor variant to wild-type Fc fragments, undesired effects (e.g., ADCC of non-target cells (non-cancer cells) that are bound by antibodies containing a wild-type Fc fragment, such as endogenous antibodies are reduced.

The efficacy of an antibody-based immunotherapy may be assessed by any method known in the art and would be evident to a skilled medical professional. For example, the efficacy of the antibody-based immunotherapy may be assessed by survival of the subject or tumor or cancer burden in the subject or tissue or sample thereof. In some embodiments, the immune cells are administered to a subject in need of the treatment in an amount effective in enhancing the efficacy of an antibody-based immunotherapy by at least 20%, e.g., 50%, 80%, 100%, 2-fold, 5-fold, 10-fold, 20-fold, 50-fold, 100-fold or more, as compared to the efficacy in the absence of the immune cells.

In some embodiments, the immune cells expressing any of the chimeric receptor variants disclosed herein are administered to a subject who has been treated or is being treated with a non-naturally occurring antibody that binds the chimeric receptor variant, such as an Fc-containing therapeutic agent (e.g., an Fc-containing therapeutic protein). The immune cells expressing any one of the chimeric receptor variants disclosed herein may be co-administered with a non-naturally occurring antibody that binds the chimeric receptor variant. For example, the immune cells may be administered to a human subject simultaneously with a non-naturally occurring antibody that binds the chimeric receptor variant. Alternatively, the immune cells may be administered to a human subject during the course of an antibody-based immunotherapy using a non-naturally occurring antibody that binds the chimeric receptor variant. In some examples, the immune cells and the non-naturally occurring antibody that binds the chimeric receptor variant can be administered to a human subject at least 4 hours apart, e.g., at least 12 hours apart, at least 1 day apart, at least 3 days apart, at least one week apart, at least two weeks apart, or at least one month apart.

Any antibody or Fc-containing protein known in the art may be modified or mutated to allow interaction of the mutated extracellular ligand-binding domain of an Fc receptor of the chimeric receptor variant with the non-naturally occurring antibody. In some embodiments, the immune cells expressing chimeric receptor variants are co-used with a non-naturally occurring antibody that binds the chimeric receptor variant to enhance the efficacy of the antibody-based immunotherapy and/or to reduce autoimmune effects. As used herein, the term “non-naturally occurring antibody” refers to an antibody or population of antibodies that does not occur in nature, e.g., an endogenous antibody of the subject. In some embodiments, the non-naturally occurring antibody has been modified or mutated relative to its wild-type counterpart, for example, having altered post-translational modification as relative to an endogenous antibody having the same amino acid sequences.

In some embodiments, the non-naturally occurring antibodies comprise one or more mutations relative to the wild-type Fc domain, such a mutation may be referred to as a compensatory mutation, which can be one or more mutations in the non-naturally occurring antibody that restores or allows interaction between the mutated antibody and the corresponding chimeric receptor variant (ACTR variant) as described herein. The non-naturally-occurring antibody may comprise one or more mutations in residues of the Fc region involved in the interaction between the Fc region and an Fc receptor to allow for interaction between the Fc region of the antibody and the mutated extracellular ligand-binding domain of an Fc receptor. In some embodiments, one or more mutations may be made in a portion of an antibody, (e.g., a therapeutic antibody) or a molecule containing an Fc domain that is involved in interaction with an Fc receptor. In some embodiments, the one or more mutations allow for interaction between the antibody and the mutated Fc binder of a chimeric receptor variant that did not occur in absence of the one or more mutations in the antibody. In some embodiments, the one or more mutations in the antibody are located in the hinge and/or CH2 domain of the antibody. Examples of mutations in the antibody known in the art and can be found, for example, in U.S. Pat. Nos. 7,601,335, 8,188,231, and 9,120,856, and include substitution mutations of amino acid residues S239, F243, R292, S298, Y300, V305, A330, I332, E333, K334, or P396 (using EU index numbering as described in Kabat et al., (1991), Sequences of Proteins of Immunological Interest, 5th Ed.). In some embodiments, the one or more mutations in the Fc fragment can be S239D, F243L, R292P, S298A, Y300L, V305I, A330L, I332E, I332D, E333A, K334A, and/or P396L. See, for example, Shields et al. J. Biol. Chem. (2001) 276(9):6591-6604; Lazar et al. Proc. Natl. Acad. Sci. USA (2006) 103(11): 4005-4010; Stavenhagen et al. Cancer Res. (2007) 67(18): 8882-8890; Isoda et al. PLoS One (2015) 10(10): e0140120; Lu et al. J. Immunol. Met. (2011) 365:132-141; Liu et al. J. Biol. Chem. (2014) 289(6): 3571-3590; and Smith et al. Proc. Natl. Acad. Sci. USA (2012) 109(16):6181-6186. See also U.S. Pat. Nos. 6,737,056, 7,662,925, 7,317,091, and 8,217,147. The relevant disclosures of the referenced publications are incorporated by reference for the purposes or subject matter referenced herein.

Examples of therapeutic antibodies comprising mutations include, without limitation ocaratuzumab (AME/Lilly), margetuximab (Macrogenics), MOR00208 (MOR/Xencor), ecromeximab (Kyowa/Life Sci. Pharma.), PF-04605412 (Pfizer/Xencor), hu14.18K322A, Adalimumab, Ado-Trastuzumab emtansine, Alemtuzumab, Basiliximab, Bevacizumab, Belimumab, Brentuximab, Canakinumab, Cetuximab, Daclizumab, Denosumab, Dinoutuzimab, Eculizumab, Efalizumab, Epratuzumab, Gemtuzumab, Golimumab, Infliximab, Ipilimumab, Labetuzumab, Natalizumab, Obinutuzumab, Ofatumumab, Omalizumab, Palivizumab, Panitumumab, Pertuzumab, Ramucirumab, Rituximab, Tocilizumab, Trastuzumab, Ustekinumab, Vedolizumab, mogamulizumab (Koywa/BioWa), obinutuzumab (Glycart/Roche), ublituximab (LFB), imgatuzumab (Glycart/Roche), BIW-8962 (Kyowa/BioWa), MDX-1401 (Medarex/BMS/BioWa), KB004 (KaloBios), ARGX-110 (arGEN-X), and ARGX-111 (arGEN-X).

In some embodiments, the therapeutic antibody comprising mutations is rituximab. Amino acid sequences of exemplary rituximab variants are provided below. In some examples, the antibody comprises an Fc domain that is identical to the Fc domain in any of SEQ ID NOs: 27-30. In one example, the Fc-containing polypeptide is an antibody comprising a heavy chain that has an amino acid sequence of any one of SEQ ID NOs: 27-30. The Fc domains, as defined by cleavage with papain, are shown in boldface. In some embodiments, the therapeutic antibody is an anti-CD19 antibody, an anti-BCMA antibody, an anti-GPC3 antibody, or trastuzumab. Amino acid sequences of exemplary variants are provided below.

Rituximab heavy chain variant 174
(SEQ ID NO: 27)
QVQLQQPGAELVKPGASVKMSCKASGYTFTSYNMHWVKQTPGRGLEWIGAIYPGNGDTSYNQKFKGKATLTADKS
SSTAYMQLSSLTSEDSAVYYCARSTYYGGDWYFNVWGAGTTVTVSAASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYVDGVEVHNAKTKPREEQY
NATYRVVSVLTVLHQDWLNGKEYKCKVSNKALPAPIAATISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEALHNHYTQKSLSLSPG
K
Rituximab heavy chain variant 175
(SEQ ID NO: 28)
QVQLQQPGAELVKPGASVKMSCKASGYTFTSYNMHWVKQTPGRGLEWIGAIYPGNGDTSYNQKFKGKATLTADKS
SSTAYMQLSSLTSEDSAVYYCARSTYYGGDWYFNVWGAGTTVTVSAASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCD
KTHTCPPCPAPELLGGPDVFLFPPKPKDTLMISRTPEVTCVVVDVSHEDPEVKFNWYVDGVEVHNAKTKPREEQY
NSTYRVVSVLTVLHQDWLNGKEYKCKVSNKALPLPEEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEALHNHYTQKSLSLSPG
K
Rituximab heavy chain variant 176
(SEQ ID NO: 29)
QVQLQQPGAELVKPGASVKMSCKASGYTFTSYNMHWVKQTPGRGLEWIGAIYPGNGDTSYNQKFKGKATLTADKS
SSTAYMQLSSLTSEDSAVYYCARSTYYGGDWYFNVWGAGTTVTVSAASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCD
KTHTCPPCPAPELLGGPDVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPEEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEALHNHYTQKSLSLSPG
K
Rituximab heavy chain variant 177
(SEQ ID NO: 30)
QVQLQQPGAELVKPGASVKMSCKASGYTFTSYNMHWVKQTPGRGLEWIGAIYPGNGDTSYNQKFKGKATLTADKS
SSTAYMQLSSLTSEDSAVYYCARSTYYGGDWYFNVWGAGTTVTVSAASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCD
KTHTCPPCPAPELLGGPSVFLLPPKPKDTLMISRTPEVTCVVVDVSHEDPEVKFNWYVDGVEVHNAKTKPPEEQY
NSTLRVVSILTVLHQDWLNGKEYKCKVSNKALPAPIEKTISKAKGQPREPQVYTLPPSRDELTKNQVSLTCLVKG
FYPSDIAVEWESNGQPENNYKTTPLVLDSDGSFFLYSKLTVDKSRWQQGNVFSCSVMHEALHNHYTQKSLSLSPG
K
Anti-CD19 light chain
(SEQ ID NO: 70)
MYRMQLLSCIALSLALVTNSDIVMTQSPATLSLSPGERATLSCRSSKSLQNVNGNTYLYWFQQKPGQSPQLLIYR
MSNLNSGVPDRFSGSGSGTEFTLTISSLEPEDFAVYYCMQHLEYPITFGAGTKLEIKRTVAAPSVFIFPPSDEQL
KSGTASVVCLLNNFYPREAKVQWKVDNALQSGNSQESVTEQDSKDSTYSLSSTLTLSKADYEKHKVYACEVTHQG
LSSPVTKSFNRGEC
Anti-CD19 heavy chain
(SEQ ID NO: 71)
MYRMQLLSCIALSLALVTNSEVQLVESGGGLVKPGGSLKLSCAASGYTFTSYVMHWVRQAPGKGLEWIGYINPYN
DGTKYNEKFQGRVTISSDKSISTAYMELSSLRSEDTAMYYCARGTYYYGTRVFDYWGQGTLVTVSSASTKGPSVF
PLAPSSKSTSGGTAALGCLVKDYFPEPVTVSWNSGALTSGVHTFPAVLQSSGLYSLSSVVTVPSSSLGTQTYICN
VNHKPSNTKVDKKVEPKSCDKTHTCPPCPAPELLGGPDVFLEPPKPKDTLMISRTPEVTCVVVDVSHEDPEVQFN
WYVDGVEVHNAKTKPREEQFNSTFRVVSVLTVVHQDWLNGKEYKCKVSNKALPAPEEKTISKTKGQPREPQVYTL
PPSREEMTKNQVSLTCLVKGFYPSDIAVEWESNGQPENNYKTTPPMLDSDGSFFLYSKLTVDKSRWQQGNVFSCS
VMHEALHNHYTQKSLSLSPGK
Wild-type anti-BCMA light chain
(SEQ ID NO: 72)
MYRMQLLSCIALSLALVTNSDIVLTQSPPSLAMSLGKRATISCRASESVTILGSHLIHWYQQKPGQPPTLLIQLA
SNVQTGVPARFSGSGSRTDFTLTIDPVEEDDVAVYYCLQSRTIPRTFGGGTKLEIKRTVAAPSVFIFPPSDEQLK
SGTASVVCLLNNFYPREAKVQWKVDNALQSGNSQESVTEQDSKDSTYSLSSTLTLSKADYEKHKVYACEVTHQGL
SSPVTKSFNRGEC
Wild-type anti-BCMA heavy chain
(SEQ ID NO: 73)
MYRMQLLSCIALSLALVTNSQIQLVQSGPELKKPGETVKISCKASGYTFTDYSINWVKRAPGKGLKWMGWINTET
REPAYAYDFRGRFAFSLETSASTAYLQINNLKYEDTATYFCALDYSYAMDYWGQGTSVTVSSASTKGPSVFPLAP
SSKSTSGGTAALGCLVKDYFPEPVTVSWNSGALTSGVHTFPAVLQSSGLYSLSSVVTVPSSSLGTQTYICNVNHK
PSNTKVDKKVEPKSCDKTHTCPPCPAPELLGGPSVFLEPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKSRWQQGNVFSCSVMHE
ALHNHYTQKSLSLSPGK
Fc-enhanced anti-BCMA heavy chain (S239D, I332E; SEQ ID NO: 74)
MYRMQLLSCIALSLALVTNSQIQLVQSGPELKKPGETVKISCKASGYTFTDYSINWVKRAPGKGLKWMGWINTET
REPAYAYDFRGRFAFSLETSASTAYLQINNLKYEDTATYFCALDYSYAMDYWGQGTSVTVSSASTKGPSVFPLAP
SSKSTSGGTAALGCLVKDYFPEPVTVSWNSGALTSGVHTFPAVLQSSGLYSLSSVVTVPSSSLGTQTYICNVNHK
PSNTKVDKKVEPKSCDKTHTCPPCPAPELLGGPDVFLEPPKPKDTLMISRTPEVTCVVVDVSHEDPEVKFNWYVD
GVEVHNAKTKPREEQYNSTYRVVSVLTVLHQDWLNGKEYKCKVSNKALPAPEEKTISKAKGQPREPQVYTLPPSR
DELTKNQVSLTCLVKGFYPSDIAVEWESNGQPENNYKTTPPVLDSDGSFFLYSKLTVDKSRWQQGNVFSCSVMHE
ALHNHYTQKSLSLSPGK
Wild-type trastuzumab light chain
(SEQ ID NO: 75)
MYRMQLLSCIALSLALVTNSDIQMTQSPSSLSASVGDRVTITCRASQDVNTAVAWYQQKPGKAPKLLIYSASFLY
SGVPSRFSGSRSGTDFTLTISSLQPEDFATYYCQQHYTTPPTFGQGTKVEIKRTVAAPSVFIFPPSDEQLKSGTA
SVVCLLNNFYPREAKVQWKVDNALQSGNSQESVTEQDSKDSTYSLSSTLTLSKADYEKHKVYACEVTHQGLSSPV
TKSFNRGEC
Wild-type trastuzumab heavy chain
(SEQ ID NO: 78)
MYRMQLLSCIALSLALVTNSEVQLVESGGGLVQPGGSLRLSCAASGFNIKDTYIHWVRQAPGKGLEWVARIYPTN
GYTRYADSVKGRFTISADTSKNTAYLQMNSLRAEDTAVYYCSRWGGDGFYAMDYWGQGTLVTVSSASTKGPSVFP
LAPSSKSTSGGTAALGCLVKDYFPEPVTVSWNSGALTSGVHTFPAVLQSSGLYSLSSVVTVPSSSLGTQTYICNV
NHKPSNTKVDKKVEPKSCDKTHTCPPCPAPELLGGPSVFLEPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDGSFFLYSKLTVDKSRWQQGNVFSCSV
MHEALHNHYTQKSLSLSPGK
Rituximab heavy chain variant S239K
(SEQ ID NO: 76)
MYRMQLLSCIALSLALVTNSQVQLQQPGAELVKPGASVKMSCKASGYTFTSYNMHWVKQTPGRGLEWIGAIYPGN
GDTSYNQKFKGKATLTADKSSSTAYMQLSSLTSEDSAVYYCARSTYYGGDWYFNVWGAGTTVTVSAASTKGPSVF
PLAPSSKSTSGGTAALGCLVKDYFPEPVTVSWNSGALTSGVHTFPAVLQSSGLYSLSSVVTVPSSSLGTQTYICN
VNHKPSNTKVDKKVEPKSCDKTHTCPPCPAPELLGGPKVFLEPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVDKSRWQQGNVFSCS
VMHEALHNHYTQKSLSLSPGK
Rituximab heavy chain variant S239K, I332E
(SEQ ID NO: 77)
MYRMQLLSCIALSLALVTNSQVQLQQPGAELVKPGASVKMSCKASGYTFTSYNMHWVKQTPGRGLEWIGAIYPGN
GDTSYNQKFKGKATLTADKSSSTAYMQLSSLTSEDSAVYYCARSTYYGGDWYFNVWGAGTTVTVSAASTKGPSVF
PLAPSSKSTSGGTAALGCLVKDYFPEPVTVSWNSGALTSGVHTFPAVLQSSGLYSLSSVVTVPSSSLGTQTYICN
VNHKPSNTKVDKKVEPKSCDKTHTCPPCPAPELLGGPKVFLEPPKPKDTLMISRTPEVTCVVVDVSHEDPEVKFN
WYVDGVEVHNAKTKPREEQYNSTYRVVSVLTVLHQDWLNGKEYKCKVSNKALPAPEEKTISKAKGQPREPQVYTL
PPSRDELTKNQVSLTCLVKGFYPSDIAVEWESNGQPENNYKTTPPVLDSDGSFFLYSKLTVDKSRWQQGNVFSCS
VMHEALHNHYTQKSLSLSPGK

Alternatively or in addition, the non-naturally occurring antibodies are modified to reduce, eliminate, or add one or more sugar moieties. In some embodiments, the non-naturally occurring antibodies are afucosylated antibodies. The terms “afucosylated” and “non-fucosylated” may be used interchangeably throughout and refer to an antibody that has reduced or absent fucosylation. In some embodiments, the non-naturally occurring antibodies are modified to add one or more additional glycosylation sites. In some embodiments, the non-naturally occurring antibodies are produced under conditions that result in altered glycosylation of the antibody.

As a set of non-limiting examples, the antibody may comprise a mutation or substitution at one or more of positions S239, F243, R292, S298, Y300, V305, A330, I332, E333, K334, and P396 of a wild-type antibody, wherein the numbering is according to the EU index. For example, the amino acid substitution may be, but is not limited to one or more of S239D, F243L, R292P, S298A, Y300L, V305I, A330L, I332E, I332D, E333A, K334A, and P396L. The antibody may be any antibody including, but not limited to, therapeutic antibodies such as an anti-CD20 antibody (e.g., Rituximab), an anti-CD19 antibody, an anti-BCMA antibody, or an anti-Her2 antibody (e.g., Trastuzumab).

An ACTR variant for use in the disclosed compositions and methods may comprise an amino acid substitution at one or more positions corresponding to 122, 134, 160, and 164 in SEQ ID NO: 18 (e.g., CD16A mutant V160Q, CD16A mutant V160W, or CD16A mutant K122L), and the Fc-containing polypeptide to be co-used with the ACTR variant may be afucosylated in its Fc domain and/or may comprise an amino acid substitution at one or more positions corresponding to S239, F243, R292, S298, Y300, V305, A330, I332, E333, K334, and P396 of a wild-type antibody.

Provided below are exemplary combinations of ACTR variants and Fc-containing polypeptide variants for co-use in any of the methods or compositions described herein. These combinations are merely illustrative and in no way limit the present disclosure:
a) an ACTR variant comprising the CD16A mutant Y134A/N164Q; and an antibody comprising (i) S239D, A330L, and I332E substitutions, or (ii) S239D and I332E substitutions as compared with the wild-type counterpart;
b) an ACTR variant comprising the CD16A mutant Y134A; and an antibody comprising (i) S239D, A330L, and I332E substitutions, or (ii) S239D and I332E substitutions as compared with the wild-type counterpart;
c) an ACTR variant comprising the CD16A mutant K122L; and an antibody comprising (i) S298A, E333A, and K334A substitutions, or (ii) F243L, R292P, Y300L, V305I, and P396L substitutions as compared with the wild-type counterpart;
d) an ACTR variant comprising the CD16A mutant V160Q; and an antibody comprising (i) S298A, E333A, and K334A substitutions, (ii) S239D, A330L, and I332E substitutions, (iii) S239D and I332E substitutions, or (iv) F243L, R292P, Y300L, V305I, and P396L substitutions as compared with the wild-type counterpart; and
e) an ACTR variant comprising the CD16A mutant V160W; and an antibody comprising (i) S298A, E333A, and K334A substitutions, (ii) S239D, A330L, and I332E substitutions, (iii) S239D and I332E substitutions, or (iv) F243L, R292P, Y300L, V305I, and P396L substitutions as compared with the wild-type counterpart.

It is appreciated in the art, that glycosylation of the Fc region of antibodies, particularly residue Asn of the CH2 domains, plays a critical role in the interaction between the Fc region and an Fc receptor. See, for example, Nose M, et al Proc Natl Acad Sci USA (1983)80:6632-6636. In some embodiments, the non-naturally occurring antibody is not glycosylated at residue Asn297. In some embodiments, the non-naturally occurring antibody is an afucosylated antibody, for example an antibody from which the fucose moieties are not present. In some embodiments, the non-naturally occurring antibody comprises mutation of one or more residue in the Fc region that is glycosylated, thereby resulting in an antibody that has reduced glycosylation or is not glycosylated.

In some embodiments, the antibody may be modified after production (e.g., post-translationally or after isolation) to reduce or eliminate the fucose moieties present on the antibody. Examples of afucosylated therapeutic antibodies include, without limitation, mogamulizumab (Koywa/BioWa), obinutuzumab (Glycart/Roche), ublituximab (LFB), imgatuzumab (Glycart/Roche), BIW-8962 (Kyowa/BioWa), MDX-1401 (Medarex/BMS/BioWa), KB004 (KaloBios), ARGX-110 (arGEN-X), and ARGX-111 (arGEN-X).

To practice the method disclosed herein, an effective amount of the immune cells expressing chimeric receptors, non-naturally occurring antibodies including Fc-containing therapeutic agents (e.g., Fc-containing therapeutic proteins such as Fc fusion proteins and therapeutic antibodies that have been modified or mutated to allow interaction with the chimeric receptors), or compositions thereof can be administered to a subject (e.g., a human cancer patient) in need of the treatment via a suitable route, such as intravenous administration. Any of the immune cells expressing chimeric receptors, non-naturally occurring antibodies including Fc-containing therapeutic agents, or compositions thereof may be administered to a subject in an effective amount. As used herein, an effective amount refers to the amount of the respective agent (e.g., the host cells expressing chimeric receptors, non-naturally occurring antibody, or compositions thereof) that upon administration confers a therapeutic effect on the subject. Determination of whether an amount of the cells or compositions described herein achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human. In some embodiments, the subject is a human cancer patient.

In some embodiments, the subject is a human patient suffering from a cancer, which can be carcinoma, lymphoma, sarcoma, blastoma, or leukemia. Examples of cancers for which administration of the cells and compositions disclosed herein may be suitable include, for example, lymphoma, breast cancer, gastric cancer, neuroblastoma, osteosarcoma, lung cancer, skin cancer, prostate cancer, colon cancer, renal cell carcinoma, ovarian cancer, rhabdomyosarcoma, leukemia, mesothelioma, pancreatic cancer, head and neck cancer, retinoblastoma, glioma, glioblastoma, and thyroid cancer.

Any of the immune cells expressing chimeric receptors described herein and/or non-naturally occurring antibodies that bind to the chimeric receptors may be prepared or administered in a pharmaceutically acceptable carrier or excipient as a pharmaceutical composition.

The phrase “pharmaceutically acceptable,” as used in connection with compositions, cells, and/or nucleic acids of the present disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions, cells, and/or nucleic acids to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20^{th} Ed. (2000) Lippincott Williams and Wilkins, Ed. K. E. Hoover.

V. Combination Treatments

The compositions and methods described in the present disclosure may be utilized in conjunction with other types of therapy for cancer, such as chemotherapy, surgery, radiation, gene therapy, and so forth. Such therapies can be administered simultaneously or sequentially (in any order) with the antibody-based immunotherapy described herein.

When co-administered with an additional therapeutic agent, suitable therapeutically effective dosages for each agent may be lowered due to the additive action or synergy.

The antibody-based immunotherapies described herein can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 41BB, OX40, etc.).

Non-limiting examples of other therapeutic agents useful for combination with antibody-based immunotherapies described herein include without limitation: (i) anti-angiogenic agents (e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000)); (ii) a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof; and (iii) chemotherapeutic compounds such as, e.g., pyrimidine analogs (5-fluorouracil, floxuridine, capecitabine, gemcitabine and cytarabine), purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In some embodiments, radiation or radiation and chemotherapy are used in combination with the antibody-based immunotherapies described herein.

For examples of additional useful agents see also Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

Kits for Therapeutic Use

The present disclosure also provides kits for use of the chimeric receptors in enhancing antibody-dependent cell-mediated cytotoxicity and/or enhancing an antibody-based immunotherapy, while reducing or preventing undesired side effects, such as autoimmunity and binding wild-type Fc fragments. Such kits may include one or more containers comprising a first pharmaceutical composition that comprises any nucleic acid or host cells (e.g., immune cells such as those described herein), and a pharmaceutically acceptable carrier, and a second pharmaceutical composition that comprises a non-naturally occurring antibody (e.g., a therapeutic antibody that has been modified or mutated to allow interaction with the chimeric receptor) and a pharmaceutically acceptable carrier.

In some embodiments, the kit can comprise instructions for use in any of the methods described herein. The included instructions can comprise a description of administration of the first and second pharmaceutical compositions to a subject to achieve the intended activity, e.g., enhancing ADCC activity, and/or enhancing the efficacy of an antibody-based immunotherapy, in a subject. The kit may further comprise a description of selecting a subject suitable for treatment based on identifying whether the subject is in need of the treatment. In some embodiments, the instructions comprise a description of administering the first and second pharmaceutical compositions to a subject who is in need of the treatment.

The instructions relating to the use of the chimeric receptors and the first and second pharmaceutical compositions described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the pharmaceutical composition is a chimeric receptor variant as described herein.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

This invention is not limited in its application to the details of construction and the arrangements of component set forth in the description herein or illustrated in the drawings. The invention is capable of other embodiments and of being practice or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items. As also used in this specification and the appended claims, the singular forms “a,” “an,” and “the” include plural references unless the context clearly dictates otherwise.

General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Antibody Binding Activity of Wild-Type ACTR and ACTR Variants

DNA sequences encoding wild-type ACTR (variant 1; SEQ ID NO: 1) and ACTR variant 26 (SEQ ID NO: 2) were generated by standard molecular cloning techniques or chemical synthesis and cloned into plasmids downstream of the T7 RNA promoter element. To generate mRNA, plasmids were linearized by restriction digestion at a site downstream of the ACTR stop codon and purified using a Qiaquick PCR Purification Kit (Qiagen; Hilden, Germany). Linearized plasmid was used as a template for mRNA generation using the T7 mScript mRNA production system (CellScript; Madison, Wis.) according to the manufacturer's instructions. Briefly, 1 μg of linearized plasmid was transcribed with T7 RNA polymerase in a 20-μL reaction volume at 37° C. for 30 min. The DNA template was digested by the addition of DNase I and subsequent incubation at 37° C. for 15 min. The reaction was purified using a MEGAclear Kit (Life Technologies; Carlsbad, Calif.) according to the manufacturer's instructions and RNA was eluted in 50 μL of H₂O. The mRNA was capped at its 5′-end with a Cap 1 structure using ScriptCap 2′-O-Methyltransferase and ScriptCap Capping enzyme in a 100-μL reaction volume for 30 min at 37° C. Subsequently, a poly (A) tail was added to the 3′-end of the capped mRNA using A-Plus Poly (A) polymerase in a 123.5-μL reaction volume at 37° C. for 30 min. The final product was purified using a MEGAclear Kit (Life Technologies; Carlsbad, Calif.) according to the manufacturer's instructions and RNA was eluted in 50 μL of H₂O. The concentration of the mRNA product was determined by measuring its absorbance at 260 nm and using a conversion factor of 40 μg/mL/absorbance unit. mRNA integrity was verified by visual inspection compared to a molecular weight marker ladder (1 kb DNA ladder, New England Biolabs; Ipswich, Mass.) after agarose gel electrophoresis using a 1.2% E-Gel with SYBR Safe DNA Gel Stain (Life Technologie; Carlsbad, Calif.).

mRNA encoding each ACTR variant was electroporated into Jurkat cells to mediate expression of the chimeric receptor variant protein on the cell surface using the Neon Transfection System (Life Technologies; Carlsbad, Calif.) and grown for 16-20 hr in a CO₂ (5%) incubator at 37° C. prior to use. Electroporated cells (3×10⁷) were harvested in a 50-mL conical tube and pelleted by centrifugation at 500×g for 5 min. The supernatant was removed by aspiration and cells were washed two times by resuspension in 5 mL of flow cytometry (FC) buffer (DPBS, 0.2% bovine serum albumin (BSA), 0.2% sodium azide), centrifugation at 500×g for 5 min, and aspiration of the supernatant. The final cell pellets were resuspended in FC buffer at a density of 3.5×10⁶ cells/mL. Jurkat cells (100 μL per well) were aliquoted into each of 12 wells per ACTR variant per binding experiment in a 96-well V-bottom plate. The plate was placed in a CO₂ (5%) incubator at 37° C. until ready for use (˜10-30 min).

Antibodies used in this experiment were CD20-specific rituximab or a low-/afucosylated form of rituximab (afucosylated rituximab). Rituxan® (Genentech; South San Francisco, Calif.) was used directly or produced by expression in HEK 293F cells (Life Technologies; Carlsbad, Calif.). For expression, two different plasmids encoding the heavy and light chains of rituximab were transduced into HEK293F cells. Antibody was purified using protein-A affinity chromatography. Afucosylated rituximab antibody was purchased from InvivoGen (San Diego, Calif.) or generated by expression in HEK 293F cells using the same procedures used for production and purification of rituximab. The cells were grown in the presence of 2F-peracetyl-fucose (Calbiochem; San Diego, Calif.), which is a fucosylation inhibitor. Afucosylated antibodies are known to mediate tighter binding to the CD16 Fc receptor when compared to their fucosylated counterparts (Shields et al, J. Biol. Chem. (2002) 277:26733-40).

A range of concentrations (0-2.47 μM) of rituximab or afucosylated rituximab was generated in FC buffer in a 96-well deep well plate. Cells were pelleted by centrifugation at 800×g for 2 min and the supernatant was removed by turning the plate upside down with a sharp, flicking motion. Antibody (100 μL) was added to each well. Binding reactions were carried out with ACTR-variant 1-bearing cells and rituximab and with ACTR-variant 26-bearing cells and rituximab or afucosylated rituximab. Binding reactions were incubated in a CO₂ (5%) incubator at 37° C. for 30 minutes.

After the binding reaction, cells were stained with antibodies for flow cytometry analysis. Cells were pelleted in the 96-well plate by centrifugation at 800×g for 2 min and the supernatant was removed by turning the plate upside down with a sharp, flicking motion. Cells were washed two times by resuspension in 100 μL of cold (4° C.) FC buffer, centrifugation at 800×g for 2 min, and removal of the supernatant, as above. Cell-bound rituximab was detected with goat F(ab′)2 anti-human IgG antibody (α-IgG-PE; Southern Biotechnology Associates; Birmingham, Ala.). Antibodies were diluted according to the manufacturer's instructions. Antibodies were incubated with cells for 10 min at room temperature in the dark, washed two times with FC buffer, as above, resuspended in a final volume of 200 μL FC buffer, and transferred to a 96-well round bottom plate for flow cytometry analysis.

Flow cytometry data was collected using an Attune NxT Acoustic Focusing Cytometer and data analysis was performed using FlowJo version 10.07 (FlowJo; Ashland, Oreg.). An inclusion gate for live cells was used and the geometric mean (GM) of fluorescence within this gate was determined at each antibody concentration. The GM values were plotted as a function of rituximab concentration.

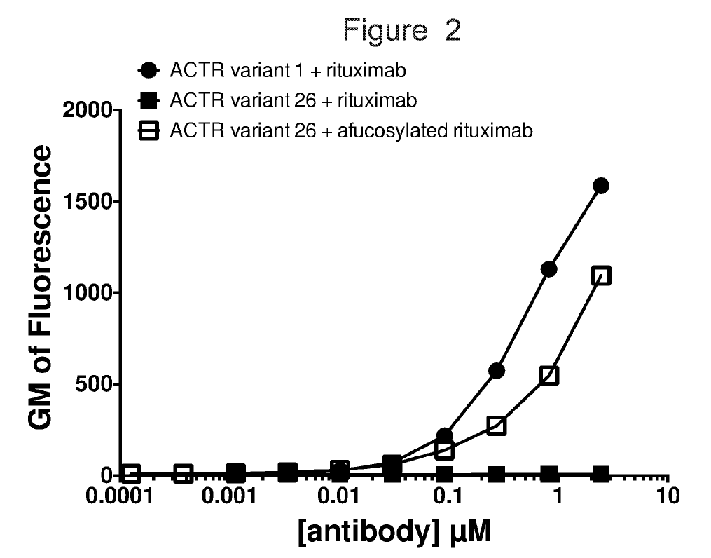

Cells expressing ACTR variant 1 bind well to rituximab while cells expressing ACTR variant 26 show no binding to rituximab at the antibody concentrations tested (FIG. 2). Cells expressing ACTR variant 26 show binding to afucosylated rituximab, demonstrating that this modification in glycosylation of the antibody restores binding to this variant ACTR sequence.

### Example 2: Jurkat Cell Activation with Various ACTR-Antibody Pairs

The ability of different ACTR-antibody pairs to activate Jurkat cells was analyzed in a reporter assay in Jurkat cells that is reflective of Jurkat cell activation. Jurkat cells were transduced with lentivirus encoding firefly luciferase downstream of a minimal CMV promoter element and tandem repeats of the nuclear factor of activated T-cells (NFAT) consensus binding site. In this cell line, upregulation of NFAT transcription factors results in binding to the transcriptional response elements and subsequent expression of luciferase, which is monitored by measuring light produce following luciferase cleavage of its substrate luciferin.

Jurkat cells with the NFAT reporter system (Jurkat-N) were electroporated with mRNAs encoding ACTR variants to mediate expression of the chimeric receptor variant protein on the cell surface using the Neon Transfection System (Life Technologies; Carlsbad, Calif.) and grown for 2 hr or 16-20 hr in a CO₂ (5%) incubator at 37° C. prior to use. Jurkat-N cells expressing the variant ACTR molecules were mixed at a 1:1 ratio with target Daudi cells expressing CD20 and varying concentrations of rituximab or afucosylated rituximab (0-66 nM) in a 100-μL reaction volume in RPMI-1640 media supplemented with 10% fetal bovine serum. Reactions were incubated for 5 hr in a CO₂ (5%) incubator at 37° C. Bright-Glo reagent (100 μL, Promega; Madison, Wis.) was added to lyse the cells and add the luciferin reagent. Reactions were incubated for 10 min in the dark and luminescence was measured using a Spectramax i3x system (Molecular Devices; Sunnyvale, Calif.) or an EnVision Multi-label plate reader (Perkin-Elmer; Waltham, Mass.). The luminescence value in the absence of antibody was considered background and was subtracted from values in the presence of antibody for each ACTR variant evaluated. Varying levels of ACTR expression was observed with the different ACTR variants, which likely influences the maximal activation signal for a given variant in a given experiment. Corrected luminescence was plotted as a function of antibody concentration and fit to the equation Y=max*X/(EC₅₀+X) where Y is the corrected luminescence, X is the antibody concentration, max is the maximum corrected luminescence, and EC₅₀ is the concentration of antibody that gives the half-maximal response.

The ability of different ACTR-antibody pairs to activate Jurkat-N cells, as measured by an increase in luminescence, was evaluated for ACTR variants 1, 26, 89, 90, 92, 93, 94, 95, 96, 97, 98, 99, 100, 101, 102, 103 (SEQ ID NOs: 1-16). These variants all contain amino acid variations within the CD16 region of the ACTR sequence at residues known to be important for CD16-antibody Fc interaction. Antibodies tested were rituximab and afucosylated rituximab.

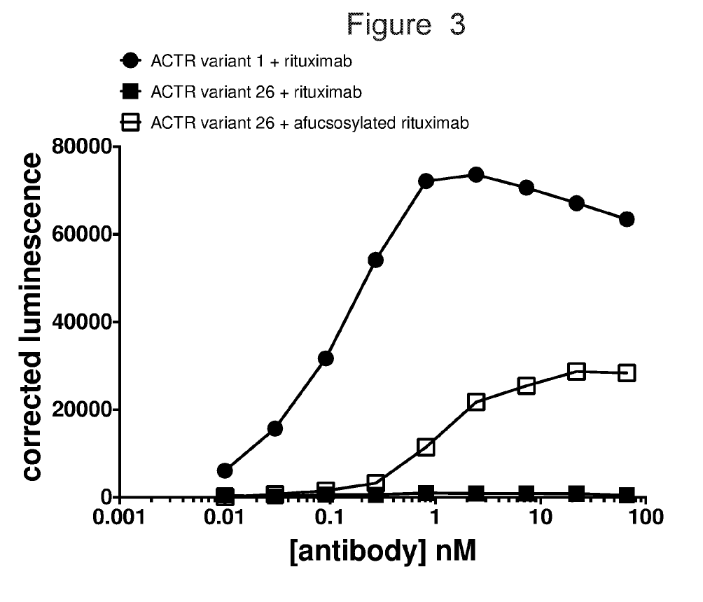

With ACTR variant 1, there is a dose-dependent increase in luminescence as a function of antibody concentration with an EC₅₀ of 0.043-0.094 nM in the presence of rituximab (FIG. 2; Table 3). With ACTR variant 26, there is no observable activation at rituximab concentrations up to 66 nM (FIG. 3), indicating an EC₅₀ of greater than 66 nM, which was the highest antibody concentration tested (Table 3); activation is observed in the presence of afucosylated rituximab (FIG. 3) with an EC₅₀ of 1.2-4.3 nM. ACTR variants 92, 93, 94, and 102 showed a similar profile with no detectable activation with rituximab (EC₅₀>66 nM; Table 3) and activation with afucosylated rituximab with EC₅₀s in the range of 0.13-1.9 nM (Table 3). ACTR variants 90, 96, 97, 98, 99, 100, and 101 showed activation with rituximab with EC₅₀s in the range of 0.17-2.7 nM and enhanced activation in the presence of afucosylated rituximab with EC₅₀s in the range of 0.020-0.071 nM (Table 3). ACTR variants 89, 95, and 103 showed no detectable activation with either rituximab or afucosylated rituximab (EC₅₀>66 nM; Table 3).

These results demonstrate that mutations in the CD16 region of ACTR that modulate the binding affinity of ACTR for antibody also modulate the ability of ACTR to activate Jurkat cells as measured by a reporter system. When incorporated into the ACTR sequence, mutations within the CD16 sequence that are at or near the interface of the CD16-Fc binding surface modulate the ability of ACTR to activate Jurkat cells. All variants that show some activation with rituximab show a concentration-shifted activation response to lower antibody concentrations in the presence of afucosylated rituximab, demonstrating that an increase in binding affinity results in an increase in activity. Some ACTR variants show no activation in the presence of rituximab at the antibody concentrations tested; with many of these variants, activation is restored in the presence of afucosylated rituximab demonstrating that an increase in binding affinity results in an increase in activity.

TABLE 3
Jurkat cell activation with different ACTR variants

| ACTR | SEQ | Representative EC₅₀ (nM) | Representative EC₅₀ (nM) |
|---|---|---|---|
| variant | ID | (rituximab) | (afucosylated rituximab) |
| 1 | 1 | 0.043-0.094 | — |
| 26 | 2 | >66 | 1.3-0 4.2 |
| 89 | 3 | >66 | >66 |
| 90 | 4 | 0.17 | 0.040 |
| 92 | 5 | >66 | 0.73 |
| 93 | 6 | >66 | 0.23 |
| 94 | 7 | >66 | 0.13 |
| 95 | 8 | >66 | >66 |
| 96 | 9 | 0.54 | 0.020 |
| 97 | 10 | 2.7 | 0.065 |
| 98 | 11 | 0.82 | 0.033 |
| 99 | 12 | 0.37 | 0.018 |
| 100 | 13 | 1.4 | 0.071 |
| 101 | 14 | 1.9 | 0.080 |
| 102 | 15 | >66 | 1.9 |
| 103 | 16 | >66 | >66 |

### Example 3: Cytotoxicity Assay

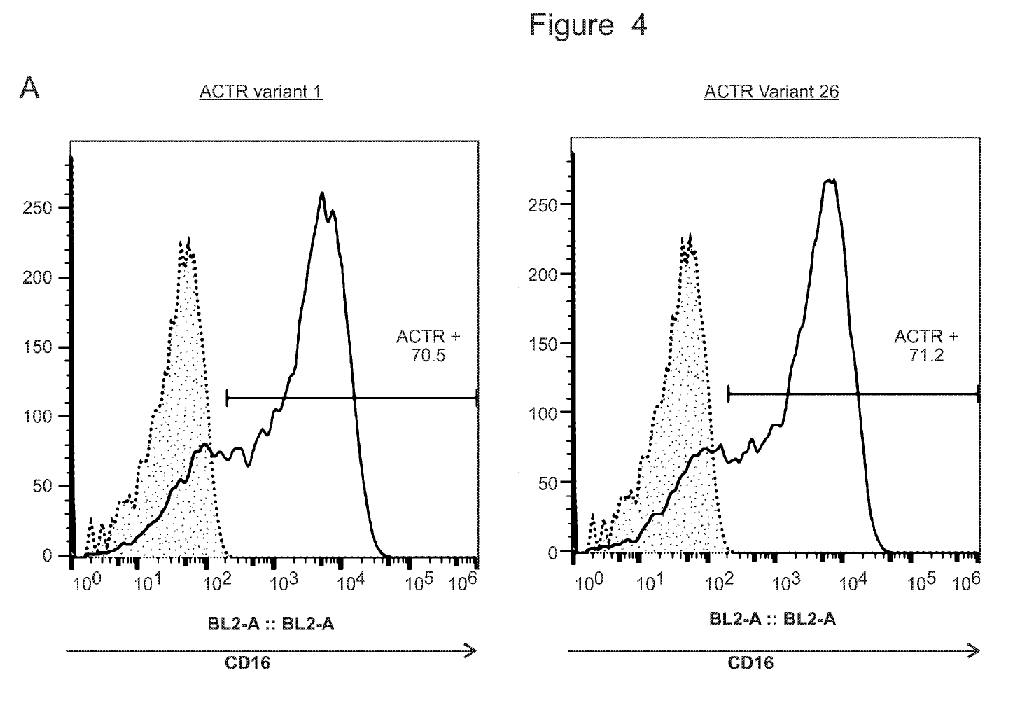

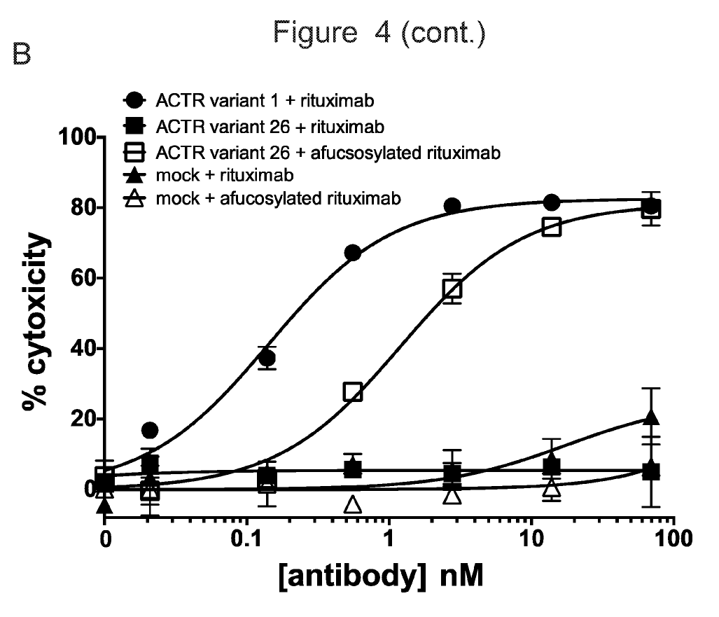

Gamma-retrovirus was generated that encoded ACTR variant 1 or ACTR variant 26. These viruses were used to infect primary human T-cells to generate cells that express ACTR variant 1 or ACTR variant 26 on their cell surface. Antibody staining for CD16 expression followed by flow cytometry demonstrated that a similar amount of ACTR was expressed in the ACTR variant 1 and ACTR variant 26 cells (FIG. 4A). These cells were used in cytotoxicity assays with CD20-positive Daudi target cells that constitutively expressed firefly luciferase and CD20-targeting rituximab or afucosylated rituximab. Mock T-cells with no ACTR were used as a control in this experiment.

T-cells (effector; E) and Daudi target cells (target; T) were incubated at a 3:1 effector-to-target ratio (30,000 target cells; 90,000 effector cells) in the presence of different concentrations of rituximab or afucosylated rituximab (0-70 nM) in a 100-μL reaction volume in RPMI 1640 media supplemented with 10% fetal bovine serum. Reactions were incubated in a CO2 (5%) incubator at 37° C. for 44 hr. A 100-μL volume of Bright-Glo luciferase assay reagent (Promega; Madison, Wis.) was added and incubated at room temperature for 10 minutes. Luminescence was measured using an Envision multilabel reader (PerkinElmer; Waltham, Mass.). The percentage of live cells was determined by dividing the luminescence signal of a given sample by the luminescence signal in the absence of antibody for each T-cell type and multiplying by 100. The percent cytotoxicity was determined by subtracting the percent live cells from 100. Percent cytotoxicity was plotted as a function of antibody concentration and fit to the equation Y=max*X/(EC50+X) where Y is the percent cytotoxicity, X is the antibody concentration, max is the maximum percent cytotoxicity, and EC50 is the concentration of antibody that gives the half-maximal response.

When target Daudi cells were incubated with mock T-cells and increasing concentrations of either rituximab or afucosylated rituximab, little to no cytotoxicity was observed (FIG. 4B). When target Daudi cells were incubated with ACTR variant 1 T-cells and increasing concentrations of rituximab, a concentration-dependent increase in cytotoxicity was observed, giving an EC50 of 0.14 nM (FIG. 4B). When target Daudi cells were incubated with ACTR variant 26 T-cells and increasing concentrations of rituximab little to no cytotoxicity was observed, similar to experiments with mock T-cells. When target Daudi cells were incubated with ACTR variant 26 T-cells and increasing concentrations of afucosylated rituximab, a concentration-dependent increase in cytotoxicity was observed giving an EC50 of 1.2 nM (FIG. 4B). These experiments demonstrate that the ACTR variant 26 is impaired for cytotoxic activity in the presence of rituximab and that activity is restored in the presence of the tighter-binding afucosylated rituximab.

### Example 4: Activation of Virally-Transduced ACTR Jurkat Cells with Fc-Mutated Antibodies

The ability of different ACTR-antibody pairs to activate Jurkat cells was analyzed in a reporter assay in Jurkat cells that is reflective of Jurkat cell activation. Jurkat cells were transduced with lentivirus encoding firefly luciferase downstream of a minimal CMV promoter element and tandem repeats of the nuclear factor of activated T-cells (NFAT) consensus binding site. In this cell line, upregulation of NFAT transcription factors results in binding to the transcriptional response elements and subsequent expression of luciferase, which is monitored by measuring light produce following luciferase cleavage of its substrate luciferin.

Jurkat cells with the NFAT reporter system (Jurkat-N) were transduced with gamma-retrovirus encoding ACTR variant 26 (SEQ ID NO: 2) to generate cells that stably expressed the ACTR variant. Jurkat-N cells expressing the variant 26 ACTR were mixed at a 1:1 ratio with target Daudi cells expressing CD20 and varying concentrations antibody (0-66 nM) in a 100-μL reaction volume in RPMI-1640 media supplemented with 10% fetal bovine serum. Reactions were incubated for 5 hr in a CO₂ (5%) incubator at 37° C. Bright-Glo reagent (100 μL, Promega; Madison, Wis.) was added to lyse the cells and add the luciferin reagent. Reactions were incubated for 10 min in the dark and luminescence was measured using a Spectramax i3x system (Molecular Devices; Sunnyvale, Calif.) or an EnVision Multi-label plate reader (Perkin-Elmer; Waltham, Mass.). The luminescence value in the absence of antibody was considered background and was subtracted from values in the presence of antibody for each ACTR variant evaluated. Varying levels of ACTR expression was observed with the different ACTR variants, which likely influences the maximal activation signal for a given variant in a given experiment. Corrected luminescence was plotted as a function of antibody concentration and fit to the equation Y=max*X/(EC₅₀+X) where Y is the corrected luminescence, X is the antibody concentration, max is the maximum corrected luminescence, and EC₅₀ is the concentration of antibody that gives the half-maximal response.

The ability of different antibodies to activate ACTR variant 26 Jurkat-N cells, as measured by an increase in luminescence, was evaluated with rituximab, afucosylated rituximab, and rituximab heavy chain Fc variants 174, 175, 176, and 177 (SEQ ID NOs: 27-30). The mutations introduced into these heavy chain Fc regions are known to enhance Fc interaction with FcγRI (CD16). Variant 174 contains mutations S298A, E333A, and K334A (Shields et al. J. Biol. Chem. (2001) 276(9): 6591-6604). Variant 175 contains mutations S239D, A330L, and I332E, and variant 176 contains mutations S239D and I332D (Lazar et al. Proc. Natl. Acad. Sci. USA (2006)103(11)). Variant 177 contains mutations F243L, R292P, Y300L, V305I, and P396L (Stavenhagen et al. Cancer Res (2007) 67(18):8882-8890). For antibody expression, two different plasmids encoding the light chain of rituximab and the rituximab heavy chain Fc variants were transduced into HEK293F cells. Antibody was purified using protein-A affinity chromatography.

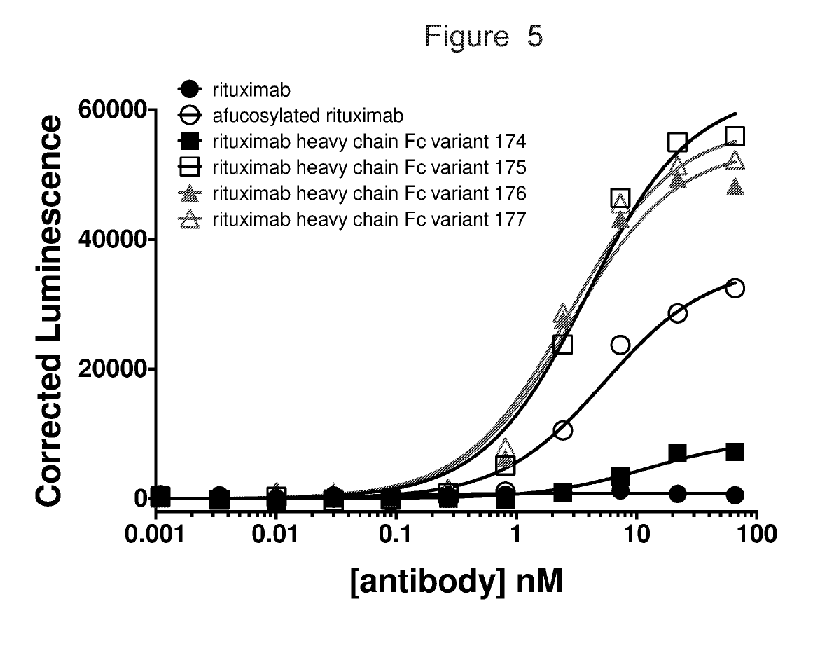

In the presence of rituximab, no activation was observed with virally-transduced ACTR variant 26 cells, similar to what was observed with mRNA-electroporated ACTR variant 26 cells (EC₅₀>66 nM; FIG. 4; Example 2). Concentration-dependent activation is observed in the presence of afucosylated rituximab, consistent with what was observed with mRNA-electroporated ACTR variant 26 cells (EC₅₀=5.4 nM; FIG. 4B; Example 2). Concentration-dependent activation was also observed in the presence of rituximab heavy chain Fc variants 175 (EC₅₀=3.9 nM), 176 (EC₅₀=2.8 nM), and 177 (EC₅₀=2.8 nM) (FIG. 5). Concentration-dependent activation was also observed in the presence of rituximab heavy chain variant 174 (EC₅₀=12 nM; FIG. 5), albeit to a much lower extent than the other Fc variants. These results demonstrated that both amino acid and carbohydrate modifications in the Fc region of an antibody that enhance interaction with wild-type Fc receptors like CD16 also restore activity.

### Example 5: Loss of Activity of ACTR Variants with Wild-Type Antibodies in Activation Assays

Gamma-retrovirus was generated that encoded ACTR variants listed in Table 1. These viruses were used to infect primary human T-cells to generate cells that express the ACTR variants on their cell surface. Antibody staining for CD16 expression followed by flow cytometry demonstrated that comparable amounts of ACTR were expressed across variants. These cells were used in activation assays with CD20-positive Daudi target cells and the CD20-targeting antibody rituximab. Mock T-cells with no ACTR were used as a control in this experiment. Activation of T-cells was evaluated by measuring T-cell release of interferon gamma (IFNγ), the increase in CD25 expression on T-cells, and the increase in CD69 expression on T-cells. CD25 and CD69 are both markers of T-cell activation and IFNγ release is increased upon T-cell activation.

T-cells (effector; E) and Daudi target cells (target; T) were incubated at a 4:1 effector-to-target ratio (60,000 target cells; 240,000 effector cells) in the presence of different concentrations of rituximab (0-2000 nM) in a 200-μL reaction volume in RPMI 1640 media supplemented with 10% fetal bovine serum. Reactions were incubated in a CO₂ (5%) incubator at 37° C. for 20-24 hr. Cells were pelleted by centrifugation and 100 μL of the supernatant was removed and frozen at −20° C. for subsequent analysis of IFNγ.

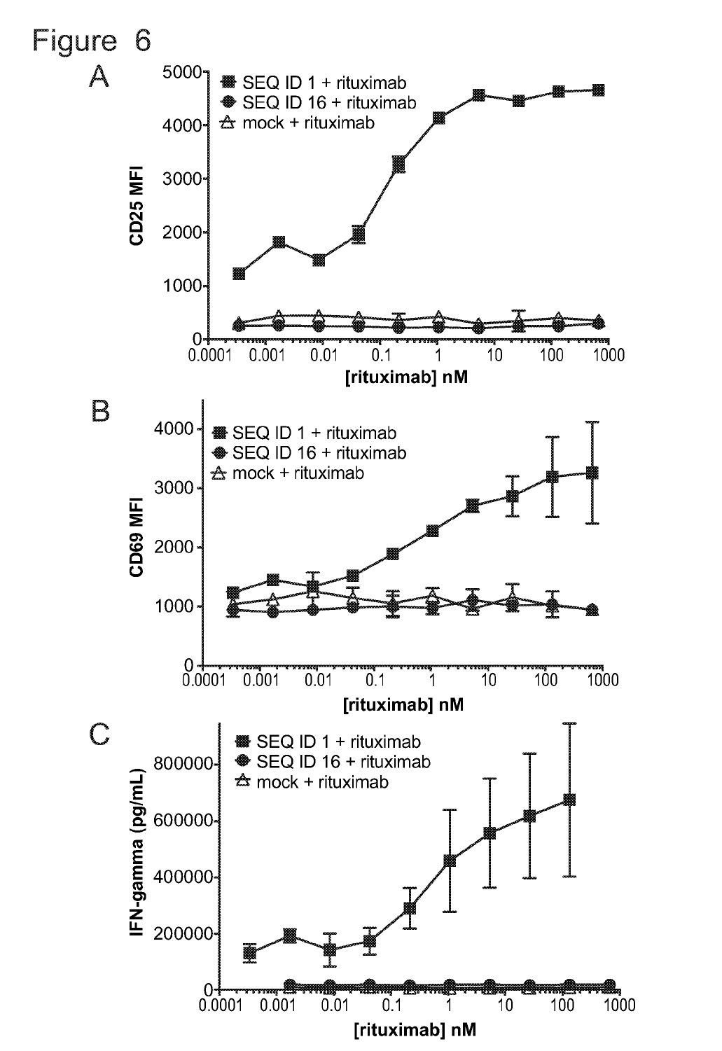

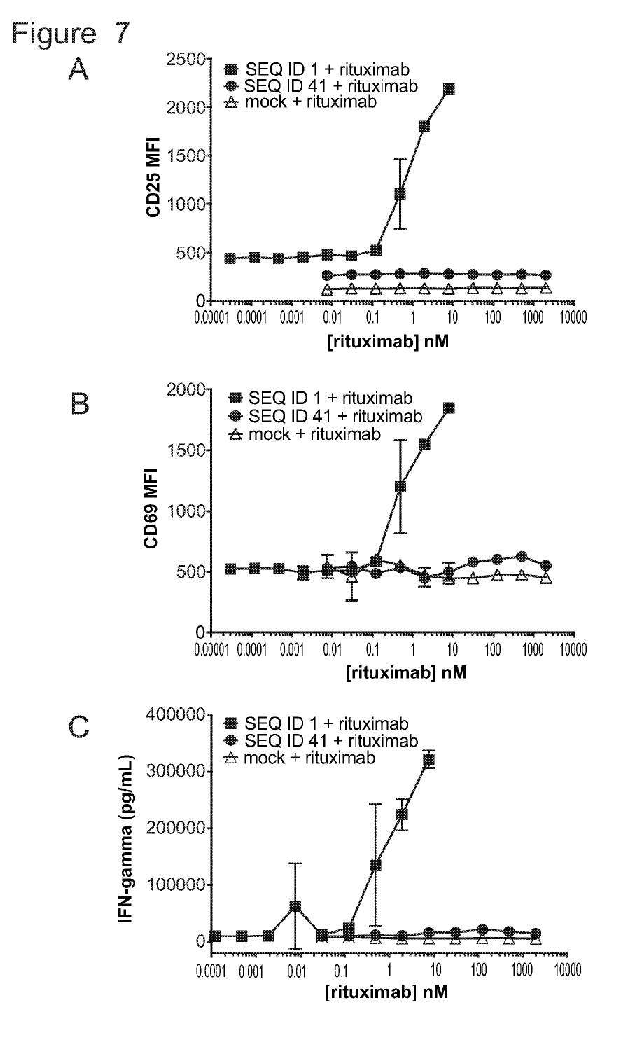

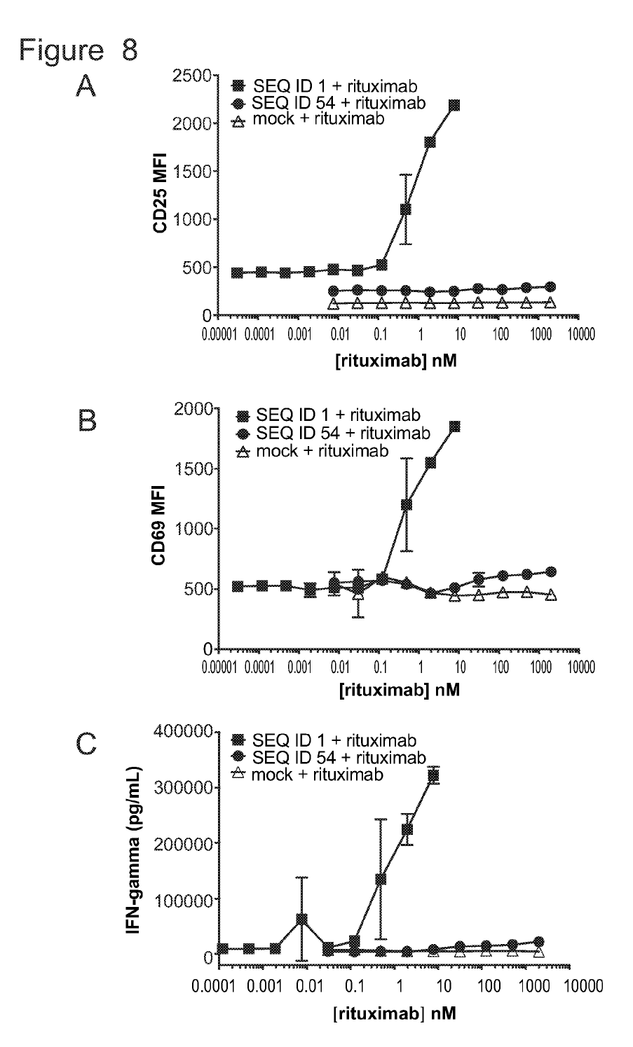

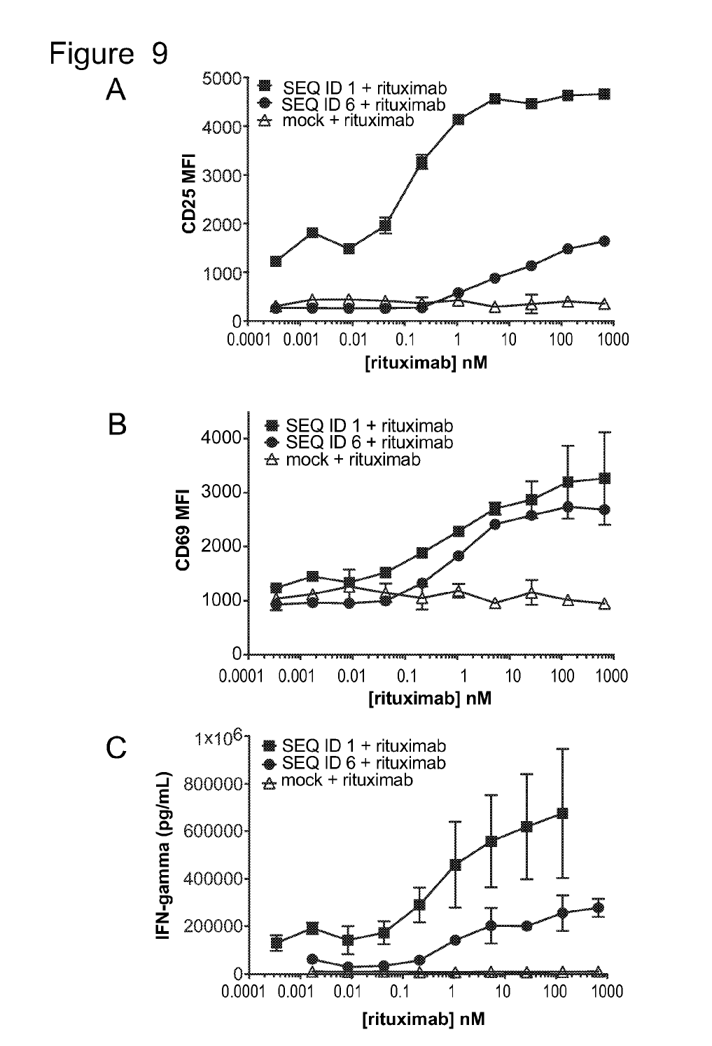

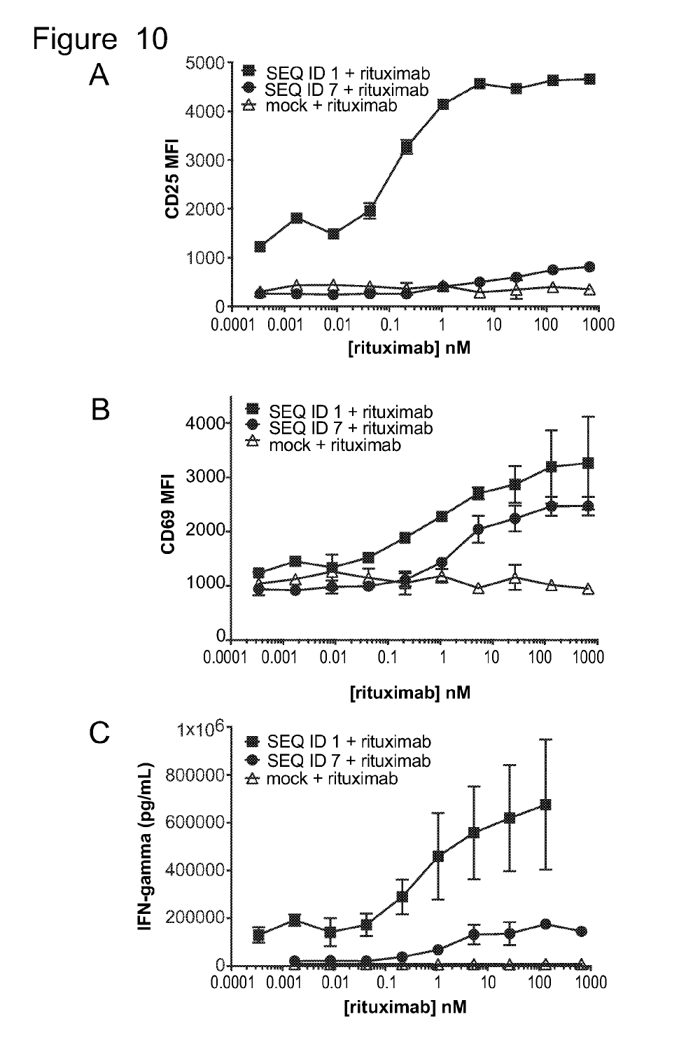

To evaluate expression of CD25 and CD69, cells were analyzed by flow cytometry. Pelleted cells were washed with DPBS and stained with fixability dye eFluor450 (Affymetrix eBioscience) for 30 min. Cells were washed with MACS buffer (autoMACS buffer plus bovine serum albumin; Miltenyi) and then stained with anti-CD3 antibody CD3 Alexa Fluor 488 Clone OKT3 (BioLegend), anti-CD16 antibody CD16 APC Clone B73.1 (BioLegend), anti-CD25 antibody CD25 PerCP Cy 5.5 Clone BC96 (Biolegend), and anti-CD69 antibody CD69 BV510 Clone FN50 (BioLegend) for 30 min on ice. Cells were washed with MACS buffer and then analyzed by flow cytometry. Flow cytometry data was analyzed using the FlowJo software package. The live, CD3+ T-cell populations were evaluated for CD25 and CD69 expression. The geometric mean of fluorescence intensity (MFI) of the CD25+ and CD69+ cells was determined within this cell population. There was low or no antibody concentration-dependent increase in CD25 and CD69 levels with ACTR variants SEQ ID NO: 16, SEQ ID NO: 41, or SEQ ID NO: 54 while ACTR variant SEQ ID 1 showed a rituximab-concentration-dependent increase (FIG. 6, panels A and B; FIG. 7, panels A and B; and FIG. 8, panels A and B). There was a modest increase in CD25 levels at higher rituximab concentrations with ACTR variant SEQ ID NO: 6 (FIG. 9, panel A) and a rituximab-concentration-dependent increase in CD69 levels was observed with ACTR variant SEQ ID NO: 6 (FIG. 9, panel B). There was low or no rituximab-concentration-dependent increase in CD25 levels with ACTR variants SEQ ID NO: 7 (FIG. 10, panel A) and a rituximab-concentration-dependent increase in CD69 levels was observed with ACTR variant SEQ ID NO: 7 (FIG. 10, panel B).

The percent change in CD25 and CD69 MFI for each variant relative to wild-type ACTR (SEQ ID NO: 1) was calculated by first subtracting the MFI of mock T-cells from the MFI of all ACTR variants, including wild-type ACTR, and then dividing the background-subtracted MFI for each variant by the background-subtracted MFI for wild-type ACTR. This number was then multiplied by 100 to give the percent relative MFI. For each variant, the value measured at the highest antibody concentration used in the experiment for both the variant and wild-type ACTR was used for the calculation; in most cases, this antibody concentration was much higher for the variant than for wild-type ACTR, whose activity is saturated at 7 nM or below. The relative CD25 and CD69 MFIs are shown in Table 4. Due to experimental variation, some values were determined to be negative due to the subtraction of the CD25 and CD69 MFI from mock cells from that measured with the ACTR variants. These differences are defined as “not significant” (N.S.) in Table 4. All variants showed a reduction in both CD25 and CD69 relative to wild-type ACTR when incubated in the presence of rituximab and CD20-expressing target cells, with the majority of variants having less than 15% of the CD25 and CD69 expression levels seen with wild-type ACTR.

TABLE 4
Activation of ACTR Variants Relative to Wild-Type
ACTR in the Presence of Rituximab

| | [antibody] | [antibody] | % | % | |
|---|---|---|---|---|---|
| ACTR | (wild-type | (variant | relative | relative | % |
| SEQ | ACTR) | ACTR) | CD25 | CD69 | relative |
| ID NO | (nM) | (nM) | MFI | MFI | IFNγ |
| mock | | | 0.00 | 0.00 | 0.00 |
| 1 | | | 100.00 | 100.00 | 100.00 |
| 2 | 133 | 667* | N.S.** | 18.40 | 3.31 |
| 3 | 133 | 667* | N.S.** | N.S.** | 0.95 |
| 5 | 133 | 667* | 1.43 | 37.14 | 18.33 |
| 6 | 133 | 667* | 29.87 | 74.90 | 40.37 |
| 7 | 133 | 667* | 10.71 | 65.65 | 20.40 |
| 8 | 133 | 667* | N.S.** | N.S.** | 3.52 |
| 15 | 133 | 667* | N.S.** | 41.40 | 13.18 |
| 16 | 133 | 667* | N.S.** | N.S.** | 1.25 |
| 33 | 7.81 | 2000 | 10.45 | 4.26 | N.S.** |
| 34 | 7.81 | 2000 | 8.15 | 3.32 | 0.94 |
| 35 | 7.81 | 2000 | 8.81 | 4.19 | 0.94 |
| 36 | 7.81 | 2000 | 11.48 | 5.44 | 1.20 |
| 37 | 7.81 | 2000 | 6.29 | N.S.** | 1.02 |
| 38 | 7.81 | 2000 | 10.54 | 3.63 | 0.46 |
| 39 | 7.81 | 2000 | 9.09 | N.S.** | 2.13 |
| 40 | 7.81 | 2000 | 12.73 | 15.89 | 5.73 |
| 41 | 7.81 | 2000 | 6.36 | 6.96 | 3.03 |
| 42 | 7.81 | 2000 | 6.02 | 8.89 | 15.46 |
| 43 | 7.81 | 2000 | 11.22 | 9.45 | 9.27 |
| 44 | 7.81 | 2000 | 15.20 | 4.44 | 0.09 |
| 45 | 125 | 2000 | 6.08 | 2.28 | 3.50 |
| 46 | 125 | 2000 | 12.34 | 5.30 | 4.17 |
| 47 | 125 | 2000 | 16.49 | 4.23 | 0.55 |
| 48 | 125 | 2000 | 12.89 | 5.07 | 0.96 |
| 49 | 125 | 2000 | 6.11 | 3.19 | 3.53 |
| 50 | 125 | 2000 | 10.08 | 5.84 | 1.71 |
| 51 | 125 | 2000 | 6.11 | 5.17 | 1.74 |
| 52 | 7.81 | 2000 | 4.66 | N.S.** | 1.49 |
| 53 | 7.81 | 2000 | 7.68 | N.S.** | N.S.** |
| 54 | 7.81 | 2000 | 7.80 | 13.53 | 5.61 |
| 55 | 7.81 | 2000 | 44.54 | 53.75 | 16.94 |
| 56 | 7.81 | 2000 | 25.55 | 24.04 | 24.49 |
| 57 | 7.81 | 2000 | 47.73 | 51.92 | 42.38 |
| 58 | 7.81 | 2000 | 11.70 | 12.09 | 12.52 |
| 59 | 7.81 | 2000 | 4.66 | 0.43 | 1.10 |
| 60 | 7.81 | 2000 | 7.05 | 1.36 | 3.76 |
| 61 | 7.81 | 2000 | 5.39 | N.S.** | N.S.** |
| 62 | 7.81 | 2000 | 9.65 | N.S.** | N.S.** |
| 63 | 125 | 2000 | 11.11 | 5.71 | 0.18 |
| 64 | 125 | 2000 | 9.87 | 6.88 | 0.14 |
| 65 | 125 | 2000 | 6.20 | 6.23 | 0.44 |
| 66 | 125 | 2000 | 9.87 | 6.71 | 0.75 |
| 67 | 80 | 2000 | N.S.** | N.S.** | 0.01 |
| 68 | 80 | 2000 | N.S.** | N.S.** | 0.11 |
| 69 | 80 | 2000 | N.S.** | N.S.** | N.S.** |

*133 nM for IFNγ values.
**N.S. represents values for which there was no significant variation as compared to mock T-cell results.

Previously-frozen supernatants were analyzed for IFNγ using the Meso Scale Discovery V-Plex Human IFNγ kit according to the manufacturer's protocol. Briefly, the Proinflammatory Panel 1 Calibrator Blend, SULFO-TAG Detection Antibody, and Read Buffer were prepared according to the manufacturer's protocol. Co-culture supernatants were thawed on ice and diluted in RP10 (RPMI 1640 with 10% fetal bovine serum) media to achieve values within the linear range of the assay. Proinflammatory calibrator blend or sample (50 μL) was added to the MSD plate. The plate was sealed, covered in foil, and incubated on a room temperature shaker for two hours at 600×g. The plate was washed three times with 150 μL phosphate buffered saline containing 0.05% Tween-20. Human IFNγ SULFO-TAG detection antibody (25 μL) was added to the plate. The plate was sealed, covered in foil, and incubated on a room temperature shaker for two hours at 600×g. The plate was washed three times with 150 μL phosphate buffered saline containing 0.05% Tween-20. Read buffer (150 μL) was added to the plate and the plates were run on the MSD Quickplex SQ 120.

Raw data was analyzed in the MSD workbench using a plate layout created for the Single Plex IFNγ MSD kits. Standard curves were adjusted to match the kit lot for each plate analyzed. Raw data in light units was extrapolated to cytokine concentration (pg/mL) using the Proinflammatory calibrator standard curve. Cytokine data were plotted as a function of antibody concentration. There was low or no rituximab-concentration-dependent increase in IFNγ levels with ACTR variants SEQ ID NO: 16, SEQ ID NO: 41, or SEQ ID NO: 54 while ACTR variant SEQ ID NO: 1 showed a rituximab-concentration-dependent increase (FIG. 6, panel C; FIG. 7, panel C; and FIG. 8, panel C). There was a modest rituximab-concentration-dependent increase in IFNγ levels with ACTR variants SEQ ID NO: 6 and SEQ ID NO: 7 at higher antibody concentrations tested (FIG. 9, panel C; FIG. 10, panel C).

The percent change in IFNγ for each variant relative to wild-type ACTR (SEQ ID NO: 1) was calculated by first subtracting the IFNγ levels of mock T-cells from the IFNγ levels of all ACTR variants, including wild-type ACTR, and then dividing the background-subtracted IFNγ levels for each variant by the background-subtracted IFNγ levels for wild-type ACTR. This number was then multiplied by 100 to give the percent relative IFNγ levels. For each variant, the value measured at the highest antibody concentration used in the experiment for both the variant and wild-type ACTR was used for the calculation; in most cases, this antibody concentration was much higher for the variant than for wild-type ACTR, whose activity is saturated at 7 nM or below. The relative IFNγ levels are shown in Table 4. Due to experimental variation, some values were determined to be negative due to the subtraction of the CD25 and CD69 MFI from mock cells from that measured with the ACTR variants. These differences are defined as “not significant” (N.S.) in Table 4. All variants showed a reduction in IFNγ levels relative to wild-type ACTR when incubated in the presence of rituximab and CD20-expressing target cells, with the majority of variants having less than 10% of the IFNγ levels expression levels seen with wild-type ACTR.

These experiments demonstrate that mutations in the CD16 region of ACTR result in a loss of T-cell activation in the presence of rituximab. The relative reduction in activity depends on the position and identity of the mutated amino acid.

### Example 6: Restoration of ACTR Variant Activity with Afucosylated Antibodies in T-Cell Assays

Gamma-retrovirus was generated that encoded ACTR variants SEQ ID NOs: 1, 5, 6, 7, 15, and 54. These viruses were used to infect primary human T-cells to generate cells that express the ACTR variants on their cell surface. These cells were used in activation assays with CD20-positive Daudi target cells and the CD20-targeting antibody rituximab and afucosylated rituximab. Activation of T-cells was evaluated by measuring T-cell release of the cytokines interferon gamma (IFNγ) and IL2.

Afucosylated rituximab was generated by transfecting two different plasmids encoding the heavy and light chains of the antibody into HEK293F cells. Cells were grown in the presence of 2F-peracetyl-fucose (Calbiochem; San Diego, Calif.), which is a fucosylation inhibitor. Afucosylated antibodies are known to mediate tighter binding to the CD16 Fc receptor when compared to their fucosylated counterparts (Shields et al, J. Biol. Chem. (2002) 277:26733-40). Antibody was purified from cell culture supernatants using protein-A affinity chromatography.

T-cells (effector; E) and Daudi target cells (target; T) were incubated at a 4:1 effector-to-target ratio (30,000 target cells; 120,000 effector cells) in the presence of different concentrations of rituximab or afucosylated rituximab (0-2000 nM) in a 200-μL reaction volume in RPMI 1640 media supplemented with 10% fetal bovine serum. Reactions were incubated in a CO₂ (5%) incubator at 37° C. for 20-24 hr. Cells were pelleted by centrifugation and 100 μL of the supernatant was removed and frozen at −20° C. for subsequent analysis of IFNγ and IL2.

Supernatants were analyzed for IFNγ and IL-2 using the Meso Scale Discovery V-Plex Human IFNγ and the V-Plex Human IL-2 kit according to the manufacturer's protocol. Briefly, the Proinflammatory Panel 1 Calibrator Blend, SULFO-TAG Detection Antibody, and Read Buffer were prepared according to the manufacturer's protocol. Co-culture supernatants were thawed on ice and diluted in RP10 (RPMI 1640 with 10% fetal bovine serum) media to achieve values within the linear range of the assay. Proinflammatory calibrator blend or sample (50 μL) was added to the MSD plate. The plate was sealed, covered in foil, and incubated on a room temperature shaker for two hours at 600×g. The plate was washed three times with 150 μL phosphate buffered saline containing 0.05% Tween-20. Human IFNγ or human IL-2 SULFO-TAG detection antibody (25 μL) was added to the plate. The plate was sealed, covered in foil, and incubated on a room temperature shaker for two hours at 600×g. The plate was washed three times with 150 μL phosphate buffered saline containing 0.05% Tween-20. Read buffer (150 μL) was added to the plate and the plates were run on the MSD Quickplex SQ 120.

Raw data was analyzed in the MSD workbench using a plate layout created for Single Plex IFNγ and Single Plex IL-2 MSD kits. Standard curves were adjusted to match the kit lot for each plate analyzed. Raw data in light units was extrapolated to cytokine concentration (pg/mL) using the Proinflammatory calibrator standard curve. Sample values were plotted in Graphpad Prism as the mean of two or three replicate values.

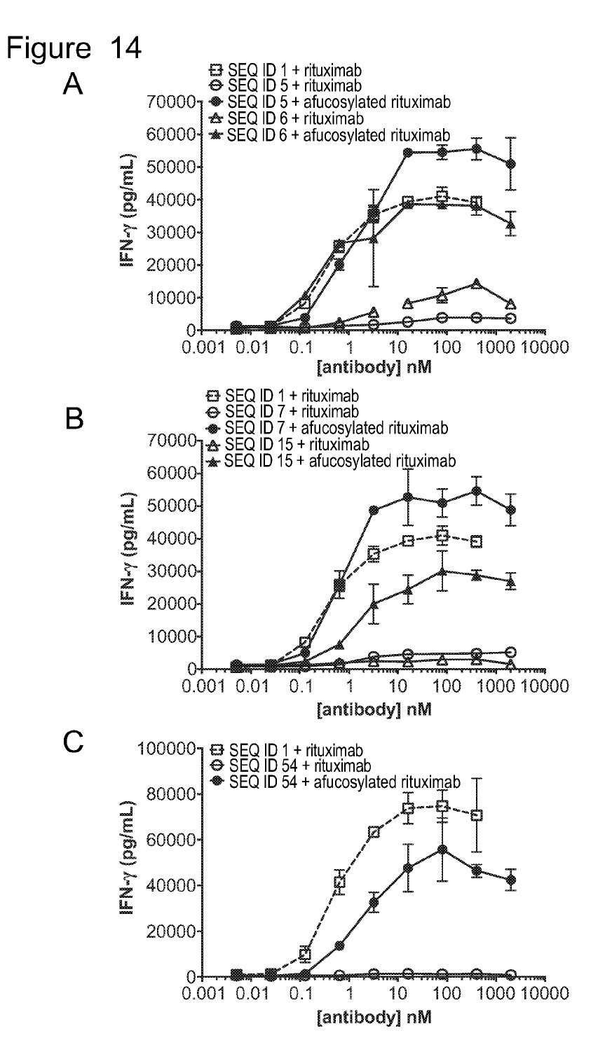

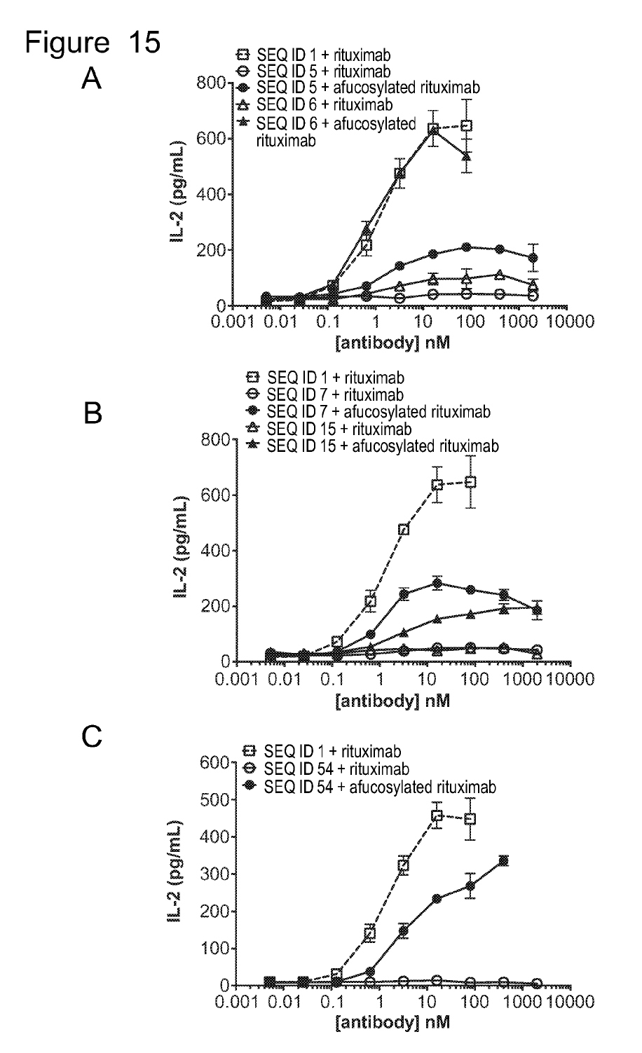

ACTR variant SEQ ID NO: 1 demonstrated a dose-dependent increase in IFNγ (FIG. 14) and IL2 (FIG. 15) as a function of rituximab concentration. ACTR variants SEQ ID NOs: 5, 6, 7, 15, and 54 show low to no increase in IFNγ (FIG. 14) or IL2 (FIG. 15) in the presence of rituximab. ACTR variants SEQ ID NOs: 5, 6, 7, 15, and 54 all demonstrated a dose-dependent increase in IFNγ (FIG. 14) as a function of afucosylated rituximab concentration. The concentration-response of IFNγ release with SEQ ID NOs: 5, 6, and 7 was similar to that observed with ACTR variant SEQ ID NO: 1 and rituximab. ACTR variants SEQ ID NOs: 5, 6, 7, 15, and 54 all demonstrated a dose-dependent increase in IL2 (FIG. 15) as a function of afucosylated rituximab concentration. The concentration-response of IL2 release with SEQ ID NO: 6 was similar to that observed with ACTR variant SEQ ID NO: 1 and rituximab.

Additionally, gamma-retrovirus was generated that encoded ACTR variants SEQ ID NOs: 1, 16, 41, and 54. These viruses were used to infect primary human T-cells to generate cells that express the ACTR variants on their cell surface. These cells were used in cytotoxicity assays with CD20-positive Daudi target cells and the CD20-targeting antibody rituximab and afucosylated rituximab.

For cytotoxicity experiments, Daudi cells that had been transduced with lentivirus encoding renilla luciferase were used; these cells constitutively expressed renilla luciferase.

T-cells (effector; E) and Daudi target cells (target; T) were incubated at a 4:1 effector-to-target ratio (30,000 target cells; 120,000 effector cells) in the presence of different concentrations of rituximab or afucosylated rituximab (0-7000 nM) in a 200-μL reaction volume in RPMI 1640 media supplemented with 10% fetal bovine serum. Reactions were incubated in a CO₂ (5%) incubator at 37° C. for 40-48 hr. Cells were pelleted by centrifugation and 2 μL of supernatant was removed for cytokine analysis. An additional 75 μL of the supernatant was removed and mixed with 75 μL of Renilla Glo substrate (Promega) according to the manufacturer's instructions. Reactions were incubated for 10 min in the dark and luminescence was measured using a Spectramax i3x system (Molecular Devices; Sunnyvale, Calif.) or an EnVision Multi-label plate reader (Perkin-Elmer; Waltham, Mass.). Percent cytotoxicity was calculated for each sample by dividing the total luminescence measured by the luminescence from a lysed sample of Daudi cells alone, subtracting this value from 1, and multiplying by 100. The percent cytotoxicity was plotted as a function of antibody concentration.

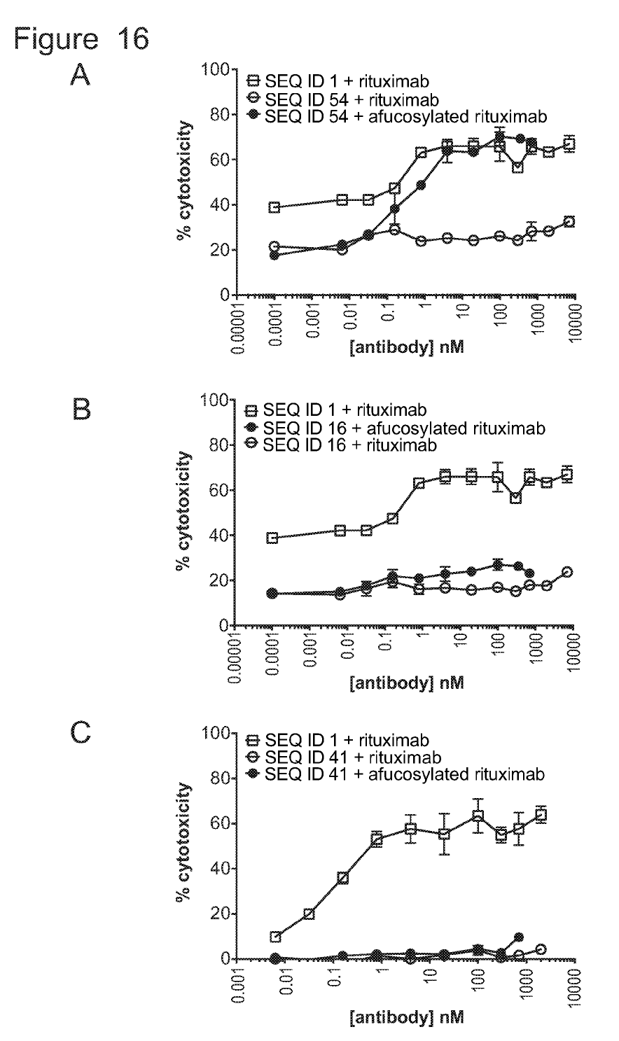

ACTR variant SEQ ID NO: 1 showed a dose-dependent increase in cytotoxicity as a function of rituximab concentration (FIG. 16). ACTR variants SEQ ID NOs: 54, 16, and 41 showed no antibody-dependent cytotoxicity in the presence of rituximab (FIG. 16). ACTR variant SEQ ID NO: 54 showed a dose-dependent increase in cytotoxicity as a function afucosylated rituximab concentration (FIG. 16, panel A) and had a concentration-response similar to that observed with ACTR variant SEQ ID NO: 1 and rituximab. ACTR variants SEQ ID NOs: 16 and 41 showed no antibody-dependent cytotoxicity in the presence of afucosylated rituximab (FIG. 16, panels B and C).

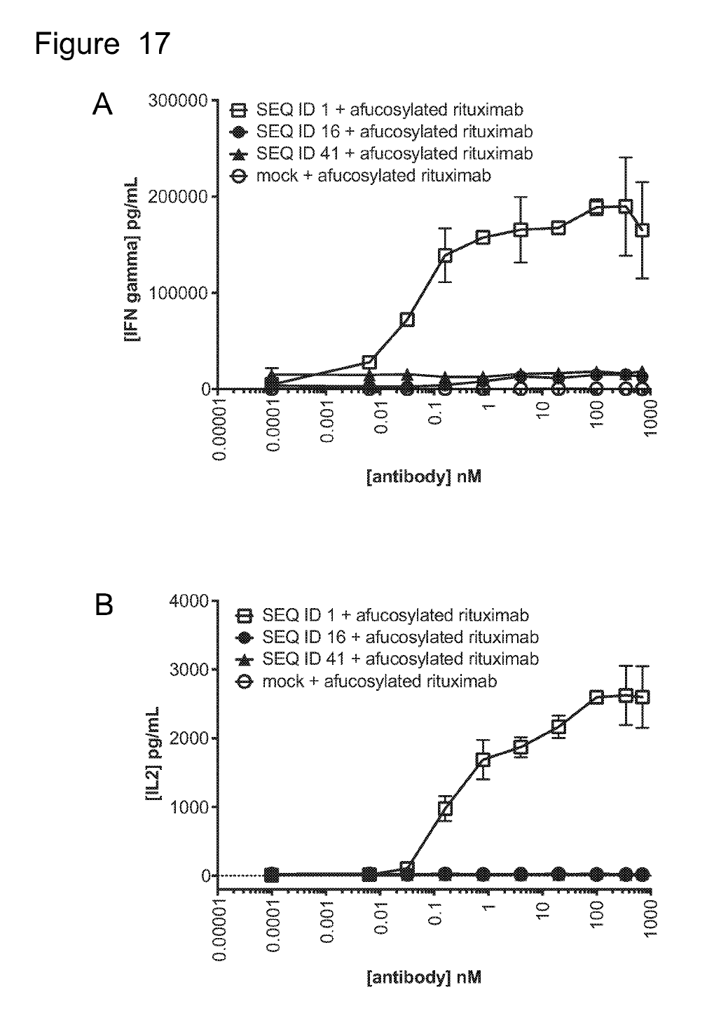

Supernatants from these reactions were also analyzed for IFNγ and IL2, as described above. ACTR variants SEQ ID NOs: 16 and 41 showed no increase in IFNγ (FIG. 17, panel A) or IL2 (FIG. 17, panel B) in the presence of rituximab or afucosylated rituximbab, consistent with cytotoxicity results observed with these variants.

T-cells expressing ACTR variants SEQ ID NOs: 1, 16, and 54 were evaluated for their ability to proliferate in the presence of CD20-expressing Raji cells and rituximab or afucosylated rituximab. Mock, untransduced T-cells were also included as a control in this experiment. Mock or ACTR T-cells were mixed at a 1:1 E:T ratio with target Raji cells (30,000 cells each) in the absence or presence of rituximab or afucosylated rituximab at varying concentrations (0-700 nM) in RPMI 1640 with 10% fetal bovine serum in a 180-μL reaction volume. After 4 days, 100 μL of media was added to each reaction. Cells were allowed to grow for a total of 7 days and then analyzed by flow cytometry. Reactions were carried out in duplicate and cells from the duplicate wells were combined for flow cytometry analysis. Reactions were stained with fixable viability dye eFluor 450 (Affymetrix) to identify dead cells, anti-CD3 antibody to identify T-cells, anti-CD16 antibody to identify surface-expressed ACTR, and anti-CD19 to identify target Raji cells. Cells were stained with dye and antibodies and resuspended in a final volume of 200 μL. Half of each reaction (100 μL) was analyzed by flow cytometry and the number of CD3+ cells was plotted as a function of antibody concentration.

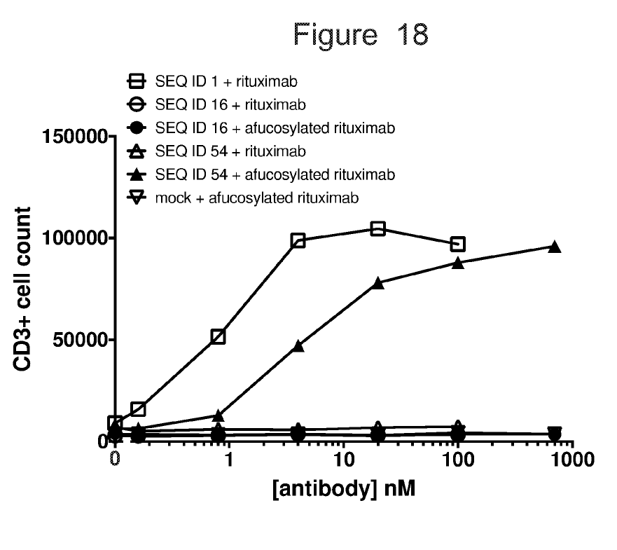

ACTR variant SEQ ID NO: 1 demonstrated antibody-dependent proliferation of CD3+ cells in the presence of rituximab and the amount of proliferation observed was concentration-dependent (FIG. 18). Mock T-cells showed no increase in the number of CD3+ cells in the presence of afucosylated rituximab. ACTR variant SEQ ID NO: 54 showed no proliferation in the presence of rituximab and demonstrated antibody- and concentration-dependent proliferation in the presence of afucosylated rituximab. ACTR variant SEQ ID NO: 16 showed no proliferation in the presence of rituximab or afucosylated rituximab.

These experiments demonstrate that T-cells bearing ACTR variants SEQ ID NOs: 5, 6, 7, 15, and 54 show low or no activity in the presence of target cells and rituximab; these ACTR variants all demonstrate antibody-dependent activity in the presence of afucosylated rituximab. These experiments also demonstrate that T-cells bearing ACTR variants SEQ ID NOs: 16 and 41 show low or no activity in the presence of either rituximab or afucosylated rituximab.

### Example 7: Restoration of ACTR Variant Activity with Fc-Enhanced Antibodies in T-Cell Assays

Gamma-retrovirus was generated that encoded ACTR variants SEQ ID NOs: 1, 16, 39, 40, 41, 42, 43, and 58. These viruses were used to infect primary human T-cells to generate cells that express the ACTR variants on their cell surface. These cells were used in activation assays with CD20-positive Daudi target cells and the CD20-targeting antibody rituximab and afucosylated rituximab. Activation of T-cells was evaluated by measuring T-cell release of the cytokines interferon gamma (IFNγ) and IL2.

Rituximab with Fc-enhancing mutations were generated by transfecting two different plasmids encoding the light chain of rituximab and heavy chain rituximab with Fc variations SEQ ID NO: 28 (S239D, A330L, I332E Fc mutations), SEQ ID NO: 29 (S239D, I332E Fc mutations), SEQ ID NO: 76 (S239K Fc mutation), and SEQ ID NO: 77 (S239K, I332E Fc mutation) into HEK293F cells. Mutations S239D, A330L, and I332E, and mutations S239D and I332D have been previously-described as Fc-enhancing mutations that increase affinity of Fc for CD16 (Lazar et al. Proc. Natl. Acad. Sci. USA (2006)103(11)). Antibodies were purified from cell culture supernatants using protein-A affinity chromatography.

T-cells (effector; E) and Daudi target cells (target; T) were incubated at a 4:1 effector-to-target ratio (30,000 target cells; 120,000 effector cells) in the presence of different concentrations of rituximab or afucosylated rituximab (0-2000 nM) in a 200-μL reaction volume in RPMI 1640 media supplemented with 10% fetal bovine serum. Reactions were incubated in a CO₂ (5%) incubator at 37° C. for 20-24 hr. Cells were pelleted by centrifugation and 100 μL of the supernatant was removed and frozen at −20° C. for subsequent analysis of IFNγ and IL2.

Supernatants were analyzed for IFNγ and IL-2 as described in Example 6. Raw data was analyzed in the MSD workbench using a plate layout created for Single Plex IFNγ and Single Plex IL-2 MSD kits. Standard curves were adjusted to match the kit lot for each plate analyzed. Raw data in light units was extrapolated to cytokine concentration (pg/mL) using the Proinflammatory calibrator standard curve. Sample values were plotted in Graphpad Prism as the mean of two or three replicate values and fit to the equation Y=max*X/(EC₅₀+X) where Y is the amount of IFNγ or IL2, X is the antibody concentration, max is the maximum value, and EC₅₀ is the concentration of antibody that gives the half-maximal response.

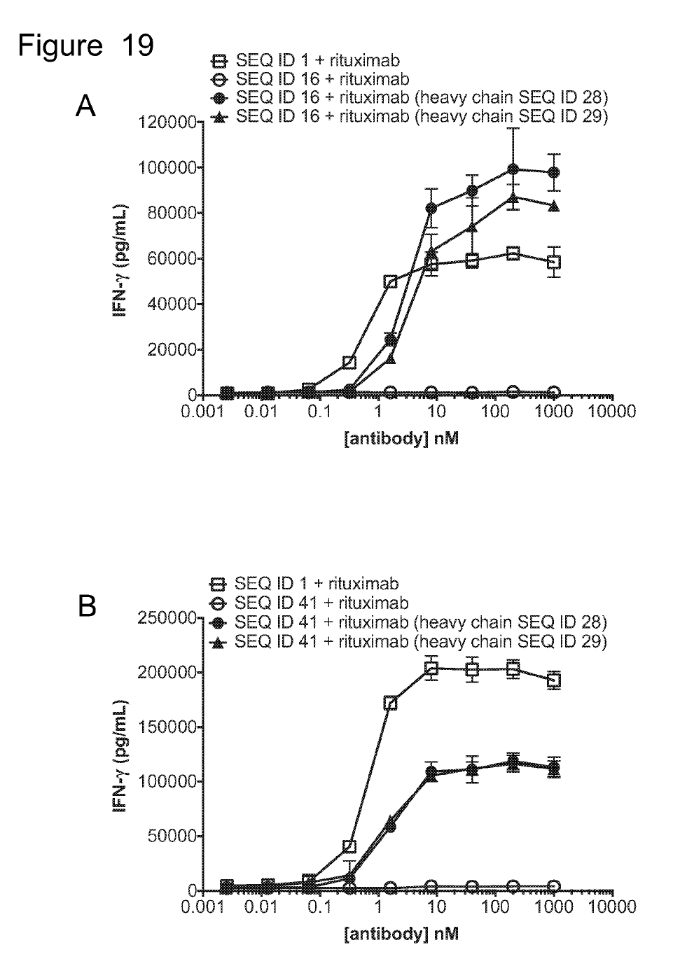

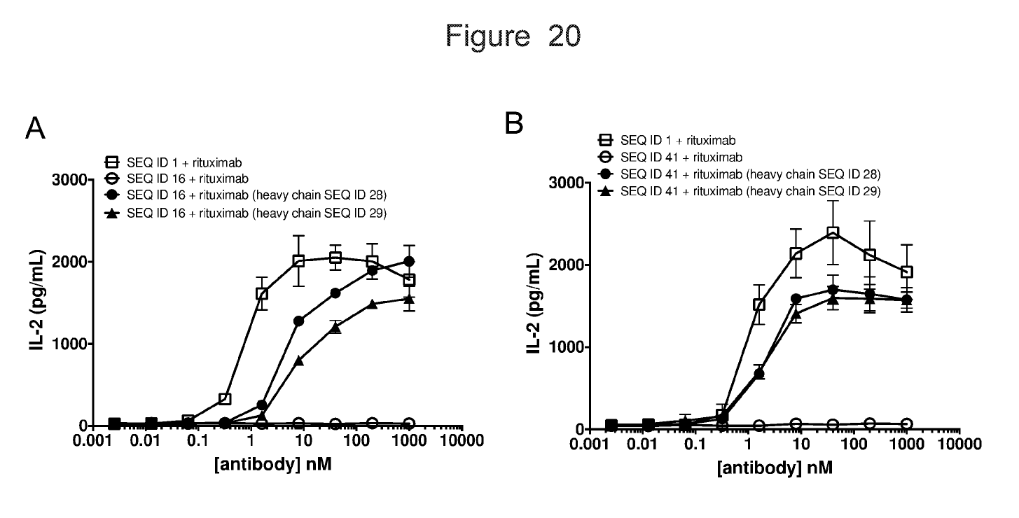

ACTR variant SEQ ID NO: 1 demonstrated a dose-dependent increase in IFNγ (FIG. 19) and IL2 (FIG. 20) as a function of rituximab concentration. ACTR variants SEQ ID NOs: 16 and 41 show low to no increase in IFNγ (FIG. 19) or IL2 (FIG. 20) in the presence of rituximab. ACTR variants SEQ ID NOs: 16 and 41 demonstrated a dose-dependent increase in IFNγ (FIG. 19) and IL2 (FIG. 20) as a function of Fc-enhanced rituximab concentration with heavy chain variants SEQ ID NOs: 28 and 29. Representative EC₅₀ and maximum values for both IFNγ and IL2 for different ACTR-antibody pairs are shown in Table 5. In instances in which there was no or low antibody concentration-response, a curve fit could not be determined. For these experiments, the EC₅₀ value is defined as not applicable (NA) and the maximum value is the IL2 amount measured at the highest antibody concentration tested (1000 nM). ACTR variants SEQ ID NOs: 16, 39, 40, 41, 42, and 43 all showed low or no activity with rituximab. All of these ACTR variants showed increased activity with one or more modified antibodies relative to activity with rituximab. ACTR variant SEQ ID NO: 58 shows some activity with rituximab but its concentration-response was significantly shifted relative to ACTR variant SEQ ID NO: 1 with rituximab. This variant shows enhanced activity with rituximab Fc variants.

TABLE 5
Representative EC₅₀ and maximum IFNγ and IL2 production with rituximab variants

| | | Value: | | | | | | | | | |
|---|---|---|---|---|---|---|---|---|---|---|---|
| | | max | | | | | | | | | |
| Antibody | | (pg/mL) | | | | | | | | | |
| Heavy | Cyto- | EC₅₀ | ACTR SEQ ID | | | | | | | | |
| Chain | kine | (nm) | mock | 1 | 16 | 39 | 40 | 41 | 42 | 43 | 58 |
| wild-type | IFNγ | max | 548* | 61542 | 1203* | 2605* | 2276* | 4052* | 3626* | 768* | 112136 |
| | | EC₅₀ | N.A. | 0.68 | N.A. | N.A. | N.A. | N.A. | N.A. | N.A. | 69.24 |
| SEQ ID | IFNγ | max | 896* | N.D | 100774 | N.D | N.D | 118638 | 102889 | 49438 | 100146 |
| NO: 28 | | EC₅₀ | N.A. | N.D | 3.40 | N.D | N.D | 1.61 | 1.55 | 3.85 | 0.35 |
| SEQ ID | IFNγ | max | 764* | N.D | 86341 | 60319 | 18575 | 116590 | 92629 | 47932 | 104227 |
| NO: 29 | | EC₅₀ | N.A. | N.D | 4.48 | 4.62 | 5.15 | 1.37 | 1.57 | 4.00 | 0.45 |
| SEQ ID | IFNγ | max | 610* | N.D | N.D | N.D | N.D | N.D | 75899 | N.D | N.D |
| NO: 76 | | EC₅₀ | N.A. | N.D | N.D | N.D | N.D | N.D | 1.79 | N.D | N.D |
| SEQ ID | IFNγ | max | 599* | N.D | N.D | N.D | N.D | N.D | 104690 | 26176 | N.D |
| NO: 77 | | EC₅₀ | N.A. | N.D | N.D | N.D | N.D | N.D | 0.56 | 3.16 | N.D |
| wild-type | IL2 | max | 35* | 2224 | 29* | 35* | 56* | 46* | 41* | 20* | 455.9 |
| | | EC₅₀ | N.A. | 1.05 | N.A. | N.A. | N.A. | N.A. | N.A. | N.A. | 7.67 |
| SEQ ID | IL2 | max | 48* | N.D | 1973 | N.D | N.D | 1712 | 2059 | 862.2 | 2146 |
| NO: 28 | | EC₅₀ | N.A. | N.D | 6.10 | N.D | N.D | 1.96 | 1.96 | 6.45 | 0.70 |
| SEQ ID | IL2 | max | 33* | N.D | 1552 | 762.5 | 416.9 | 1634 | 1967 | 763.8 | 2041 |
| NO: 29 | | EC₅₀ | N.A. | N.D | 9.32 | 8.60 | 7.92 | 1.94 | 1.89 | 5.79 | 0.91 |
| SEQ ID | IL2 | max | 35* | N.D | N.D | N.D | N.D | N.D | 1327 | N.D | N.D |
| NO: 76 | | EC₅₀ | N.A. | N.D | N.D | N.D | N.D | N.D | 2.69 | N.D | N.D |
| SEQ ID | IL2 | max | 33* | N.D | N.D | N.D | N.D | N.D | 2103 | 292 | N.D |
| NO: 77 | | EC₅₀ | N.A. | N.D | N.D | N.D | N.D | N.D | 0.68 | 4.89 | N.D |

N.D = not determined
N.A. = not applicable, no curve could be determined
*= maximum value defined as value at 1000 nM antibody

Gamma-retrovirus was generated that encoded ACTR variants SEQ ID NOs: 5, 7, 15, 16, and 54. These viruses were used to infect primary human T-cells to generate cells that express the ACTR variants on their cell surface. These cells were used in activation assays with CD20-positive Daudi target cells and the CD20-targeting antibody rituximab and afucosylated rituximab. Activation of T-cells was evaluated by measuring T-cell release of the cytokines interferon gamma (IFNγ) and IL2.

Rituximab antibodies with Fc-enhancing mutations were generated by transfecting two different plasmids encoding the light chain of rituximab and heavy chain rituximab with Fc variations SEQ ID NO: 27 (298A, E333A, K334A Fc mutations), SEQ ID NO: 28 (S239D, A330L, I332E Fc mutations), SEQ ID NO: 29 (S239D, I332E Fc mutations), and SEQ ID NO: 30 (F243L, R292P, Y300L, V305I, P396L Fc mutations) into HEK293F cells. Mutations S298A, E333A, and K334A have been previously-described as Fc-enhancing mutations that increase affinity of Fc for CD16 (Shields et al. J. Biol. Chem. (2001) 276(9): 6591-6604). Mutations S239D, A330L, and I332E, and mutations S239D and I332D have been previously-described as Fc-enhancing mutations that increase affinity of Fc for CD16 (Lazar et al. Proc. Natl. Acad. Sci. USA (2006)103(11)). Mutations F243L, R292P, Y300L, V305I, and P396L have been previously-described as Fc-enhancing mutations that increase affinity of Fc for CD16 (Stavenhagen et al. Cancer Res (2007) 67(18):8882-8890). Cells were grown in the absence or presence of 2F-peracetyl-fucose (Calbiochem; San Diego, Calif.), which is a fucosylation inhibitor. Afucosylated antibodies are known to mediate tighter binding to the CD16 Fc receptor when compared to their fucosylated counterparts (Shields et al, J. Biol. Chem. (2002) 277:26733-40). Antibody was purified from cell culture supernatants using protein-A affinity chromatography.

T-cells (effector; E) and Daudi target cells (target; T) were incubated at a 4:1 effector-to-target ratio (30,000 target cells; 120,000 effector cells) in the presence of different concentrations of rituximab or afucosylated rituximab (0-100 nM) in a 200-μL reaction volume in RPMI 1640 media supplemented with 10% fetal bovine serum. Reactions were incubated in a CO₂ (5%) incubator at 37° C. for 20-24 hr. Cells were pelleted by centrifugation and supernatant was removed and frozen at −20° C. for subsequent analysis of IL2.

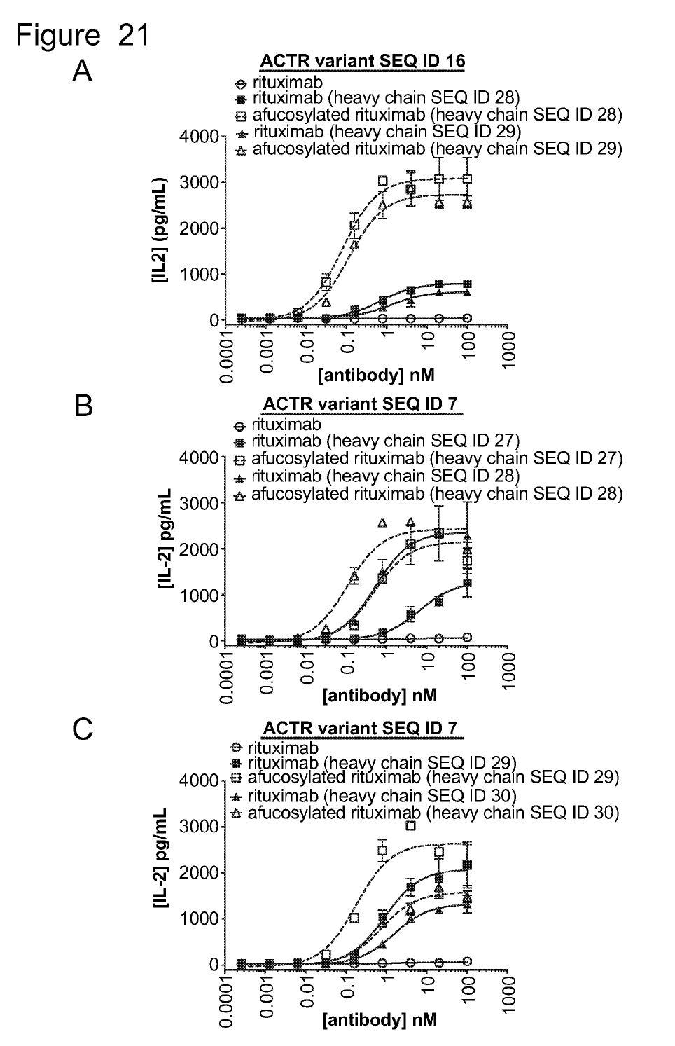

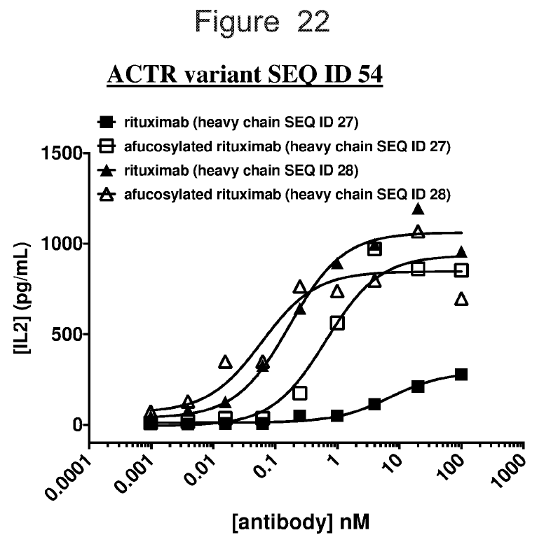

Supernatants were analyzed for IL-2 and data was analyzed as described in Example 6. ACTR variants SEQ ID NOs: 7 and 16 show low to no increase in IL2 (FIG. 21) in the presence of rituximab while ACTR variant SEQ ID NO: 1 shows a concentration-dependent increase in IL2. ACTR variant SEQ ID NO: 16 demonstrated a concentration-dependent increase in IL2 as a function of Fc-enhanced rituximab concentration with heavy chain variants SEQ ID NOs: 28 and 29 and the concentration-response was further enhanced when afucosylated versions of these antibodies were tested (FIG. 21, panel A). ACTR variant SEQ ID NO: 7 demonstrated a concentration-dependent increase in IL2 as a function of Fc-enhanced rituximab concentration with heavy chain variants SEQ ID NOs: 27, 28, 29, and 30 and the concentration-response was further enhanced when afucosylated versions of these antibodies were tested (FIG. 21, panels B and C). ACTR variant SEQ ID NO: 54 has shown no or low IL2 production in the presence of rituximab in previous experiments (FIG. 15, panel C). ACTR variant SEQ ID NO: 54 demonstrated a concentration-dependent increase in IL2 as a function of Fc-enhanced rituximab concentration with heavy chain variant SEQ ID NO: 27 and the concentration-response was further enhanced when the afucosylated version of this antibody was tested (FIG. 22). ACTR variant SEQ ID NO: 54 demonstrated a concentration-dependent increase in IL2 as a function of Fc-enhanced rituximab concentration with heavy chain variant SEQ ID NO: 28 and the concentration-response was slightly enhanced when the afucosylated version of this antibody was tested (FIG. 22). Representative EC₅₀ and maximum values for IL2 for different ACTR-antibody pairs are shown in Table 6. In instances in which there was no or low antibody concentration-response, a curve fit could not be determined. For these experiments, the EC₅₀ value is defined as not applicable (NA) and the maximum value is the IL2 amount measured at the highest antibody concentration tested (100 nM). ACTR variants SEQ ID NOs: 5, 7, 15, and 16 all showed low or no activity with rituximab. All of these ACTR variants showed increased activity with one or more modified antibodies relative to activity with rituximab. Activity was further-enhanced in the presence of afucosylated versions of these antibodies.

TABLE 6
Representative EC₅₀ and maximum IL2 production with
fucosylated and afucosylated rituximab variants

| | | rituximab heavy chain variant | | | | | | | | |
|---|---|---|---|---|---|---|---|---|---|---|
| | | | | afuco- | | afuco- | | afuco- | | afuco- |
| ACTR | | | | sylated | | sylated | | sylated | | sylated |
| variant | | Wild- | SEQ | SEQ | SEQ | SEQ | SEQ | SEQ | SEQ | SEQ |
| SEQ ID | Value | type | ID 27 | ID 27 | ID 28 | ID 28 | ID 29 | ID 29 | ID 30 | ID 30 |
| 5 | max | 48* | 597 | 1214 | 67* | 1809 | ND | ND | ND | ND |
| | (pg/mL) | | | | | | | | | |
| | EC₅₀ | NA | 41.59 | 0.36 | NA | 1.02 | ND | ND | ND | ND |
| | (nM) | | | | | | | | | |
| 7 | max | 77* | 1245 | 2173 | 2375 | 2474 | 2081 | 2683 | 1321 | 1576 |
| | (pg/mL) | | | | | | | | | |
| | EC₅₀ | NA | 6.54 | 0.50 | 0.53 | 0.11 | 0.92 | 0.18 | 1.52 | 0.74 |
| | (nM) | | | | | | | | | |
| 15 | max | 68* | ND | ND | 2320 | 2685 | 2596 | 2494 | ND | ND |
| | (pg/mL) | | | | | | | | | |
| | EC₅₀ | NA | ND | ND | 0.32 | 0.15 | 0.25 | 0.21 | ND | ND |
| | (nM) | | | | | | | | | |
| 16 | max | 40* | ND | ND | 760 | 3086 | 572 | 2751 | ND | ND |
| | (pg/mL) | | | | | | | | | |
| | EC₅₀ | NA | ND | ND | 0.71 | 0.08 | 1.29 | 0.11 | ND | ND |
| | (nM) | | | | | | | | | |
| 54 | max | ND | 280 | 943 | 1025 | 780 | ND | ND | ND | ND |
| | (pg/mL) | | | | | | | | | |
| | EC₅₀ | ND | 7.07 | 0.66 | 0.17 | 0.061 | ND | ND | ND | ND |
| | (nM) | | | | | | | | | |

ND = not determined
NA = not applicable, no curve could be determined
*= maximum value defined as value at 100 nM antibody

For cytotoxicity experiments, Daudi cells that had been transduced with lentivirus encoding renilla luciferase were used; these cells constitutively expressed renilla luciferase.

T-cells expressing ACTR variants SEQ ID NOs: 1, 16, 41 and 54 were evaluated. Mock, untransduced T-cells were used as a control in this experiment. T-cells (effector; E) and Daudi target cells (target; T) were incubated at a 4:1 effector-to-target ratio (30,000 target cells; 120,000 effector cells) in the presence of different concentrations of rituximab or afucosylated rituximab with heavy chain variant SEQ ID NO: 29 (0-100 nM) in a 200-μL reaction volume in RPMI 1640 media supplemented with 10% fetal bovine serum. Reactions were incubated in a CO₂ (5%) incubator at 37° C. for 40-48 hr. Cells were pelleted by centrifugation and supernatant was removed. Renilla Glo substrate (100 μL; Promega) was added to each pellet according to the manufacturer's instructions. Reactions were incubated for 10 min in the dark and luminescence was measured using a Spectramax i3x system (Molecular Devices; Sunnyvale, Calif.) or an EnVision Multi-label plate reader (Perkin-Elmer; Waltham, Mass.). Percent cytotoxicity was calculated for each sample by dividing the total luminescence measured by the luminescence from a lysed sample of Daudi cells alone, subtracting this value from 1, and multiplying by 100. The percent cytotoxicity was plotted as a function of antibody concentration.

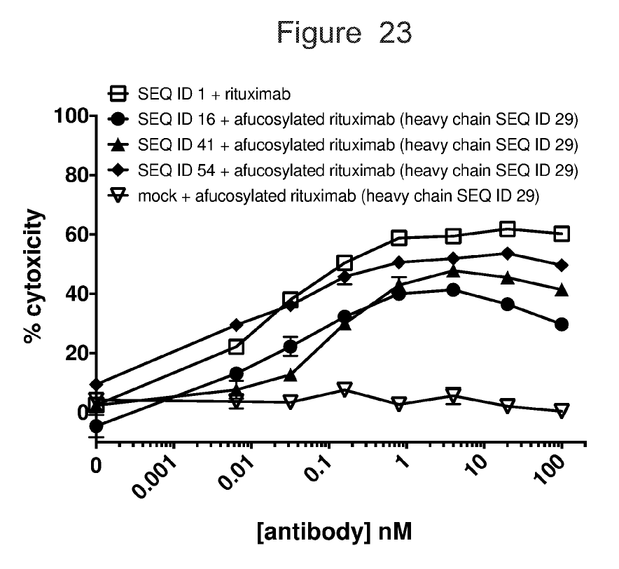

ACTR variant SEQ ID NO: 1 showed a dose-dependent increase in cytotoxicity as a function of rituximab concentration (FIG. 23). Previous experiments with ACTR variants SEQ ID NOs: 16, 41, and 54 have demonstrated no or low antibody-dependent cytotoxicity with rituximab (FIG. 16). ACTR variants SEQ ID NOs: 16, 41, and 54 showed a dose-dependent increase in cytotoxicity as a function afucosylated rituximab with heavy chain variant SEQ ID NO: 29 concentration (FIG. 23) and had a concentration-response similar (SEQ ID NO: 54) or slightly shifted (SEQ ID NOs: 16 and 41) relative to that observed with ACTR variant SEQ ID NO: 1 and rituximab. ACTR variant SEQ ID NO: 41, which bears the N164Q mutation, is expected to be relatively insensitive to the enhanced activity with afucosylated antibodies gained by variants without this mutation (Ferrara et al., J. Biol. Chem. (2006) 81: 5032-36). As such, the activity of this variant in the presence of afucosylated rituximab with heavy chain variant SEQ ID NO: 29 is likely due to the Fc-enhancing amino acid mutations in SEQ ID NO: 29 and not the fact that the antibody is afucosylated.

T-cells expressing ACTR variants SEQ ID NOs: 1, 16, 41, and 54 were evaluated for their ability to proliferate in the presence of CD20-expressing Raji cells and rituximab, Fc-enhanced rituximab variants, or afucosylated Fc-enhanced rituximab variants. ACTR T-cells were mixed at a 1:1 E:T ratio with target Raji cells (30,000 cells each) in the absence or presence of antibody at varying concentrations (0-100 nM) in RPMI 1640 with 10% fetal bovine serum in a 180-μL reaction volume. After 4 days, 100 μL of media was added to each reaction. Cells were allowed to grow for a total of 7 days and then analyzed by flow cytometry. Reactions were carried out in duplicate and cells from the duplicate wells were combined for flow cytometry analysis. Reactions were stained with fixable viability dye eFluor 450 (Affymetrix) to identify dead cells, anti-CD3 antibody to identify T-cells, anti-CD16 antibody to identify surface-expressed ACTR, and anti-CD19 to identify target Raji cells. Cells were stained with dye and antibodies and resuspended in a final volume of 200 μL. Half of each reaction (100 μL) was analyzed by flow cytometry and the number of CD3+ cells was plotted as a function of antibody concentration.

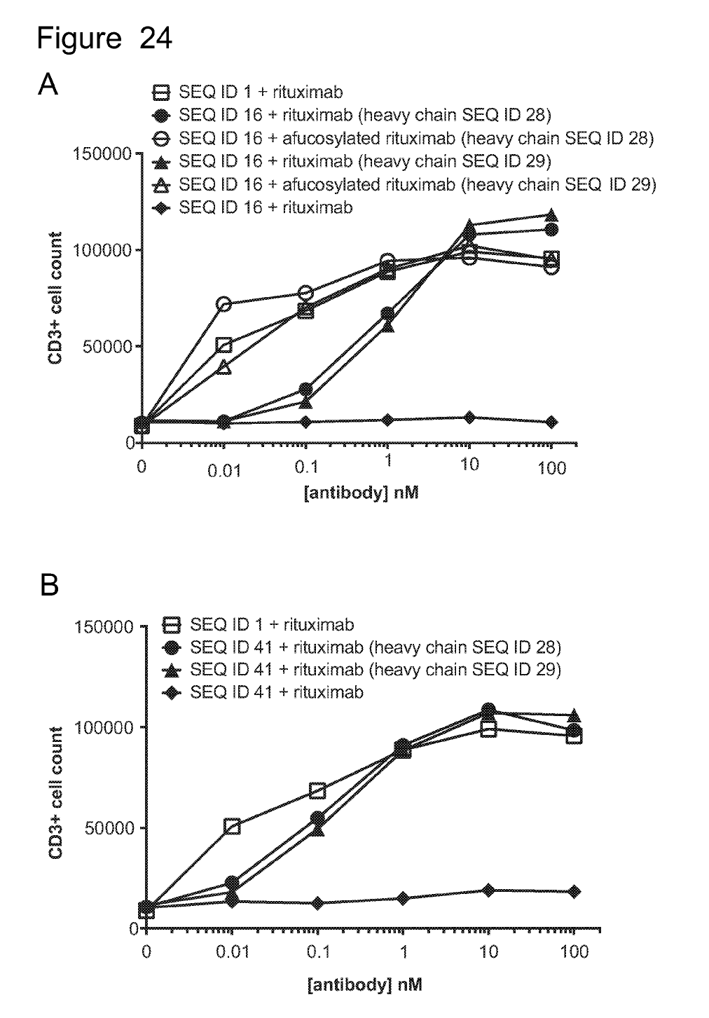

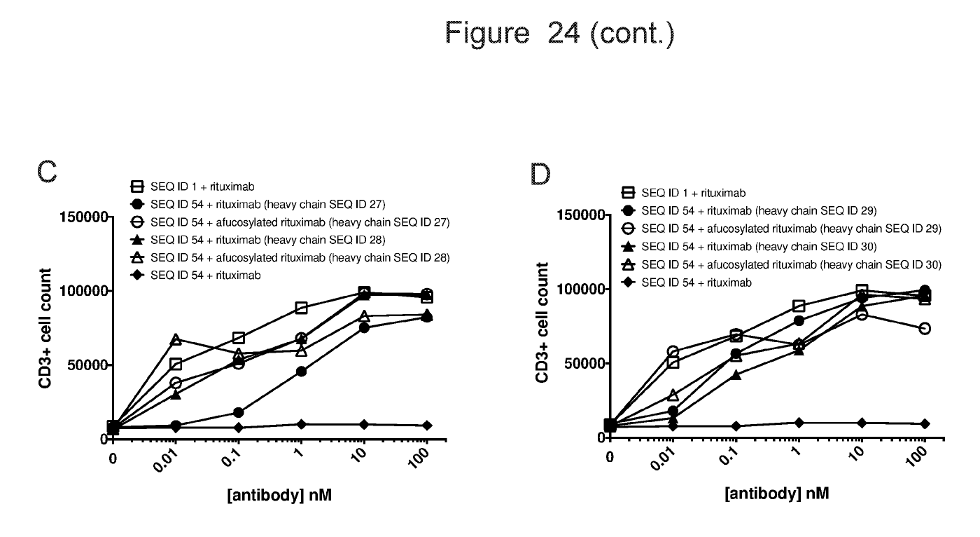

ACTR variant SEQ ID NO: 1 demonstrated antibody-dependent proliferation of CD3+ cells in the presence of rituximab and the amount of proliferation observed was concentration-dependent (FIG. 24). ACTR variant SEQ ID NO: 16 showed no proliferation in the presence of rituximab and demonstrated antibody- and concentration-dependent proliferation in the presence of fucosylated and afucosylated rituximab variants with heavy chains SEQ ID NOs: 28 and 29 (FIG. 24, panel A). The concentration-response was shifted to lower concentrations in the presence of the afucosylated versions of these antibodies relative to the fucosylated versions and was similar to that observed with ACTR variant SEQ ID NO: 1 and rituximab. ACTR variant SEQ ID NO: 41 showed no proliferation in the presence of rituximab and demonstrated antibody- and concentration-dependent proliferation in the presence of rituximab variants with heavy chains SEQ ID NOs: 28 and 29 (FIG. 24, panel B). The concentration-response for both of these antibodies was similar to that observed with ACTR variant SEQ ID NO: 1 and rituximab. ACTR variant SEQ ID NO: 54 showed no proliferation in the presence of rituximab and demonstrated antibody- and concentration-dependent proliferation in the presence of fucosylated and afucosylated rituximab variants with heavy chains SEQ ID NOs: 27, 28, 29, and 30 (FIG. 24, panels C and D). The concentration-response was shifted to lower concentrations in the presence of the afucosylated version of the antibody with rituximab heavy chain variant SEQ ID NO: 27 relative to the fucosylated version of this antibody. The concentration-response with the other fucosylated and afucosylated antibodies was similar to each other and to that observed with ACTR variant SEQ ID NO: 1 and rituximab.

Activation of T-cells was evaluated by measuring increase in CD25 expression on T-cells, and the increase in CD69 expression on T-cells, which are both markers of T-cell activation, in the presence of Fc-enhanced antibodies and target cells. For these experiments ACTR variants SEQ ID NOs: 1, 16, and 54 were evaluated with rituximab, afucosylated rituximab, and/or afucosylated rituximab with heavy chain variant SEQ ID NO: 29. T-cells (effector; E) and Daudi target cells (target; T) were incubated at a 4:1 effector-to-target ratio (60,000 target cells; 240,000 effector cells) in the presence of different concentrations of antibody (0-7000 nM) in a 200-μL reaction volume in RPMI 1640 media supplemented with 10% fetal bovine serum. Reactions were incubated in a CO₂ (5%) incubator at 37° C. for 20-24 hr. Cells were pelleted by centrifugation.

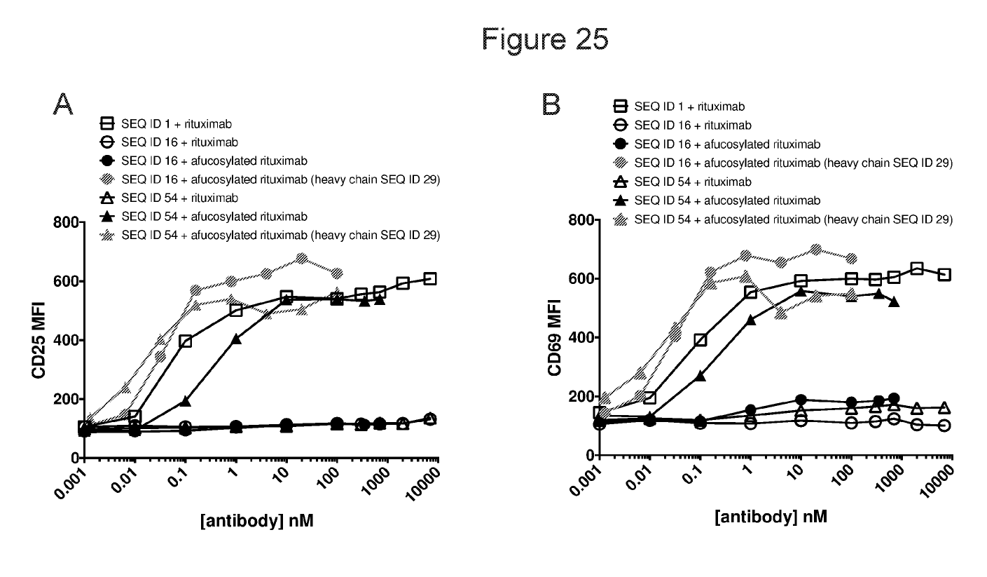

To evaluate expression of CD25 and CD69, cells were analyzed by flow cytometry. Pelleted cells were washed with DPBS and stained with fixability dye eFluor450 (Affymetrix eBioscience) for 30 min. Cells were washed with MACS buffer (autoMACS buffer plus bovine serum albumin; Miltenyi) and then stained with anti-CD3 antibody CD3 Alexa Fluor 488 Clone OKT3 (BioLegend), anti-CD16 antibody CD16 APC Clone B73.1 (BioLegend), anti-CD25 antibody CD25 PerCP Cy 5.5 Clone BC96 (Biolegend), and anti-CD69 antibody CD69 BV510 Clone FN50 (BioLegend) for 30 min on ice. Cells were washed with MACS buffer and then analyzed by flow cytometry. The flow cytometry data was analyzed using the FlowJo software package. The live, CD3+ T-cell populations were evaluated for CD25 and CD69 expression. The geometric mean of fluorescence intensity (MFI) of the CD25+ and CD69+ cells was determined within this cell population for each ACTR variant. A concentration-dependent increase in both CD25 and CD69 levels was observed with ACTR variant SEQ ID NO: 1 in the presence of rituximab (FIG. 25). There was low or no antibody concentration-dependent increase in CD25 and CD69 levels for ACTR variants SEQ ID NO: 16 and SEQ ID NO: 54 in the presence of rituximab or for ACTR variant SEQ ID NO: 16 in the presence of afucosylated rituximab (FIG. 25). ACTR variant SEQ ID NO: 54 shows an antibody concentration-dependent increase in CD25 and CD69 levels in the presence of afucosylated rituximab, which is shifted to slightly higher concentrations than that observed with ACTR variant SEQ ID NO: 1 in the presence of rituximab (FIG. 25). ACTR variants SEQ ID NO: 16 and SEQ ID NO: 54 show an antibody concentration-dependent increase in CD25 and CD69 levels in the presence of afucosylated rituximab with heavy chain variant SEQ ID NO: 29, which is shifted to slightly lower concentrations than that observed with ACTR variant SEQ ID NO: 1 in the presence of rituximab (FIG. 25).

These experiments demonstrate that T-cells bearing ACTR variants SEQ ID NOs: 5, 7, 15, 16, 39, 40, 41, 42, 43, 54 and 58 show low or no activity in the presence of target cells and rituximab; these ACTR variants all demonstrate antibody-dependent activity in the presence of Fc-enhanced rituximab heavy chain variants SEQ ID NO: 27, SEQ ID NO: 28, SEQ ID NO: 29, and/or SEQ ID NO: 30. For some of these ACTR variants, activity is further-enhanced in the presence of afucosylated versions of Fc-enhanced rituximab heavy chain variants SEQ ID NO: 27, SEQ ID NO: 28, SEQ ID NO: 29, and/or SEQ ID NO: 30.

### Example 8: Restoration of ACTR Variant Activity with Afucosylated and Fc-Enhanced Anti-BCMA Antibodies

The ability of different ACTR-antibody pairs to activate Jurkat cells was analyzed in a reporter assay in Jurkat cells that is reflective of Jurkat cell activation. Jurkat cells were transduced with lentivirus encoding firefly luciferase downstream of a minimal CMV promoter element and tandem repeats of the nuclear factor of activated T-cells (NFAT) consensus binding site to generate Jurkat-N cells. In this cell line, upregulation of NFAT transcription factors results in binding to the transcriptional response elements and subsequent expression of luciferase, which is monitored by measuring light produce following luciferase cleavage of its substrate luciferin.

Gamma-retroviruses encoding ACTR variants (SEQ ID NOs: 1, 2, 6, 7, 16, and 54) were each transduced into Jurkat N cells. Antibodies used in this experiment were derived from antibody C11D5.3 (SEQ ID NO: 3, variable heavy chain, and SEQ ID NO: 4, variable light chain from application WO 2010/104949 A2, the relevant disclosures of which are incorporated by reference herein). These variable regions were combined with constant heavy chain or kappa light chain human antibody regions, as appropriate, without or with heavy chain mutations S239D and I332E, to generate SEQ ID NO: 72 (light chain), SEQ ID NO: 73 (heavy chain), and SEQ ID NO: 74 (S239D, I332E heavy chain). Heavy chain mutations S239D and I332E have been shown to enhance antibody Fc interactions with CD16 (Lazar et al. Proc. Natl. Acad. Sci. USA (2006)103: 4005-10). Four different anti-BCMA antibodies were generated: wild-type (light chain SEQ ID NO: 72 and heavy chain SEQ ID NO: 73), afucosylated (light chain SEQ ID NO: 72 and heavy chain SEQ ID NO: 73), Fc-enhanced (light chain SEQ ID NO: 72 and heavy chain SEQ ID NO: 74), and afucosylated Fc-enhanced (light chain SEQ ID NO: 72 and heavy chain SEQ ID NO: 74). For expression of each antibody, two different plasmids encoding the appropriate heavy and light chains were transduced into HEK293F cells. For generation of afucosylated antibodies, cells were grown in the presence of 2F-peracetyl-fucose (Calbiochem; San Diego, Calif.), which is a fucosylation inhibitor. Afucosylated antibodies are known to mediate tighter binding to the CD16 Fc receptor when compared to their fucosylated counterparts (Shields et al, J. Biol. Chem. (2002) 277:26733-40). Antibodies were purified from cell culture supernatants using protein-A affinity chromatography.

Jurkat-N cells expressing the variant ACTR molecules were mixed at a 1:1 ratio with target H929 cells expressing BCMA and varying concentrations of anti-BCMA antibodies (0-133 nM) in a 100-μL reaction volume in RPMI-1640 media supplemented with 10% fetal bovine serum. Reactions were incubated for 5 hr in a CO₂ (5%) incubator at 37° C. Bright-Glo reagent (100 μL, Promega; Madison, Wis.) was added to lyse the cells and add the luciferin reagent. Reactions were incubated for 10 min in the dark and luminescence was measured using a Spectramax i3x system (Molecular Devices; Sunnyvale, Calif.) or an EnVision Multi-label plate reader (Perkin-Elmer; Waltham, Mass.). The luminescence value in the absence of antibody was considered background and was subtracted from values in the presence of antibody for each ACTR variant evaluated. Corrected luminescence was plotted as a function of antibody concentration and fit to the equation Y=max*X/(EC₅₀+X) where Y is the corrected luminescence, X is the antibody concentration, max is the maximum corrected luminescence, and EC₅₀ is the concentration of antibody that gives the half-maximal response. For curves that were saturated or near-saturated at the highest antibody concentrations used in these experiments, EC₅₀ values were determined using the equation described above. For curves that were not saturated, the EC₅₀ values were approximated by assuming the maximal value measured in the experiment for that ACTR variant with the afucosylated Fc-enhanced antibody was the maximal achievable signal and estimating the concentration of antibody at half-maximal signal. For curves with no measurable signal, EC₅₀ values were not determined (ND). Curves for which there was some detectable signal at higher antibody concentrations but for which EC₅₀ could not be approximated are indicated as “detectable signal”.

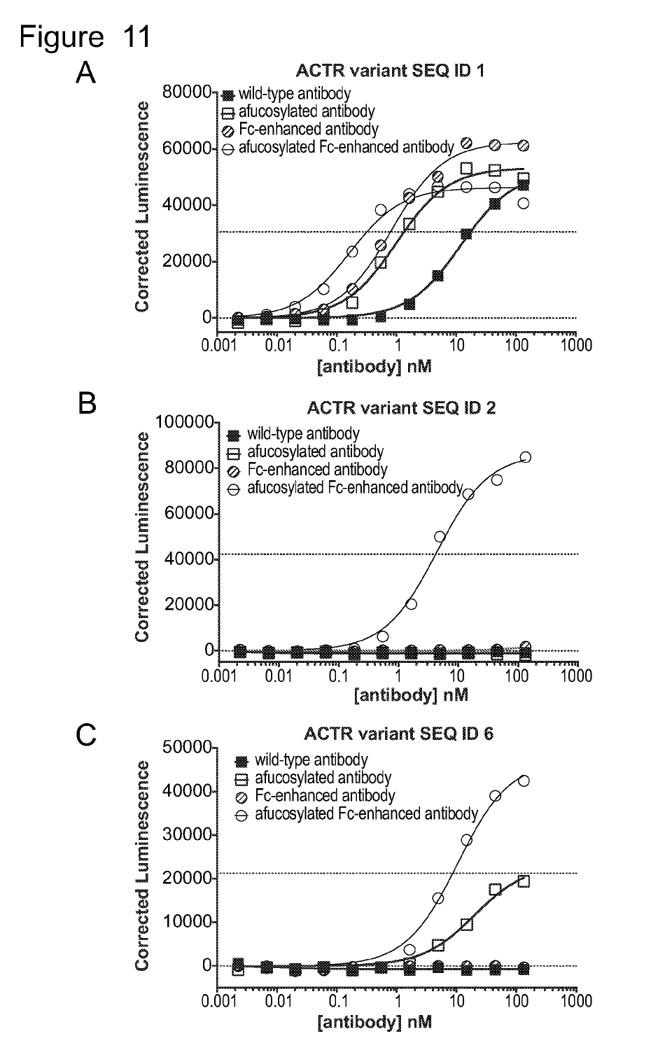

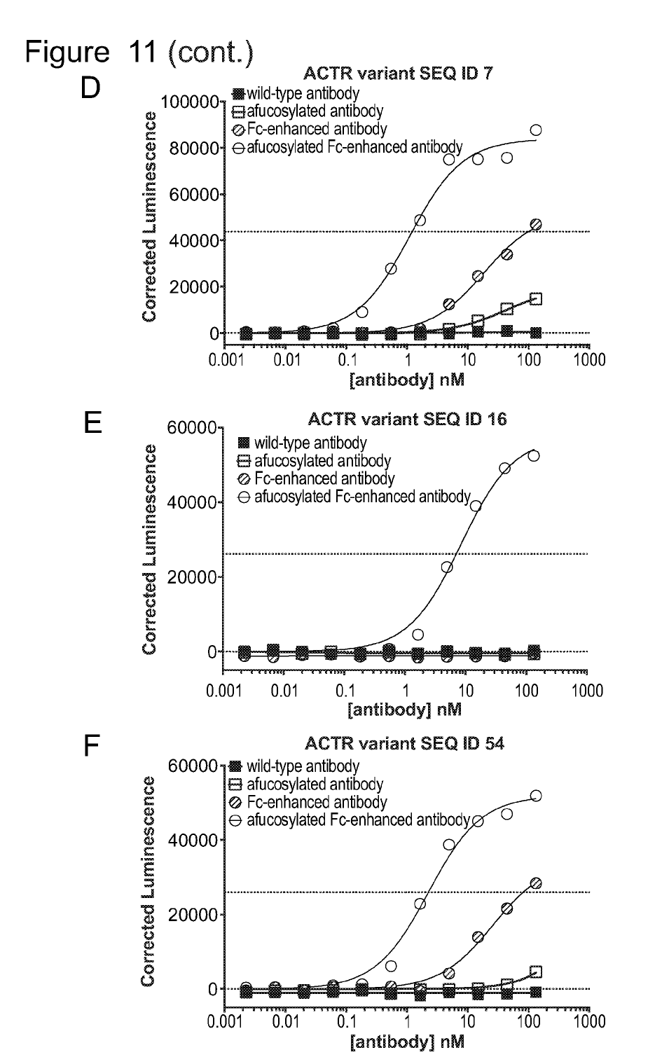

The ability of different ACTR-antibody pairs to activate Jurkat-N cells, as measured by an increase in luminescence, was evaluated for ACTR variants SEQ ID NOs: 2, 6, 7, 16, and 54. These variants all contain amino acid variations within the CD16 region of the ACTR sequence at residues known to be important for CD16-antibody Fc interaction. ACTR variant SEQ ID NO: 1 was also evaluated; this variant has no variations within its CD16 region of the ACTR sequence relative to CD16-V158 sequence (SEQ ID NO: 18). Antibodies tested were wild-type anti-BCMA antibody, afucosylated anti-BCMA antibody, Fc-enhanced anti-BCMA antibody, and afucosylated Fc-enhanced anti-BCMA antibody. With ACTR variant SEQ ID NO: 1, there was a dose-dependent increase in luminescence as a function of antibody concentration in the presence of all four antibodies tested (FIG. 11, panel A). The EC₅₀ value was lowest in the presence of the afucosylated Fc-enhanced antibody, followed by the Fc-enhanced antibody, the afucosylated antibody, and the wild-type antibody (Table 7). ACTR variants SEQ ID NOs: 2, 6, 7, 16, and 54 showed no activity with the wild-type antibody at the antibody concentrations tested in this experiment (FIG. 11). ACTR variant SEQ ID NO: 6 showed good activity with afucosylated antibody, ACTR variants SEQ ID NOs: 7 and 54 showed detectable activity with afucosylated antibody at the highest antibody concentrations tested, and ACTR variants SEQ ID NOs: 2 and 16 showed no detectable activity with afucosylated antibody at the antibody concentrations tested in this experiment (FIG. 11, Table 7). ACTR variants SEQ ID NOs: 7 and 54 showed good activity with Fc-enhanced antibody and ACTR variants SEQ ID NOs: 2, 6, and 16 showed no detectable activity with Fc-enhanced antibody at the antibody concentrations tested in this experiment (FIG. 11, Table 7). ACTR variants SEQ ID NOs: 2, 6, 7, 16, and 54 all showed good activity with the afucosylated Fc-enhanced antibody with EC₅₀ values below that observed with SEQ ID NO: 1 and the wild-type antibody (FIG. 11, Table 7).

TABLE 7
Activation of ACTR Variants with Anti-BCMA Antibody Variants

| | Representative EC₅₀ (nM) | | | |
|---|---|---|---|---|
| ACTR | | | | afucosylated |
| variant | wild-type | afucosylated | Fc-enhanced | Fc-enhanced |
| SEQ ID 1 | <20 | 1.00 | 0.83 | 0.17 |
| SEQ lD 2 | ND | ND | ND | 4.36 |
| SEQ lD 6 | ND | <100 | ND | <5 |
| SEQ lD 7 | ND | detectable | <100 | 1.12 |
| | | signal | | |
| SEQ ID 16 | ND | ND | ND | 8.44 |
| SEQ ID 54 | ND | detectable | <100 | 2.26 |
| | | signal | | |

These results demonstrate that mutations in the CD16 region of ACTR that are at or near the interface of the CD16-Fc binding surface modulate the ability of ACTR to activate Jurkat cells. All variants tested in these experiments show no activation with wild-type anti-BCMA antibody, while the ACTR sequence with the wild-type CD16-V158 sequence (SEQ ID NO: 1) shows robust activity in the presence of this antibody. All variants have restored activity with one or more of the modified antibodies tested (afucosylated, Fc-enhanced, and/or afucosylated Fc-enhanced) demonstrating that activity of these variants can be restored with antibodies that have increased affinity for CD16.

### Example 9: Restoration of ACTR Variant Activity with an Fc-Enhanced Anti-CD19 Antibody

Jurkat-N cells were generated as described in Example 8. Gamma-retroviruses encoding ACTR variants (SEQ ID NOs: 1, 2, 5, 6, 15, 16, 39, 40, 41, 43, 54, and 58) were each transduced into Jurkat N cells. The anti-CD19 antibody used in these experiments was derived from U.S. Pat. No. 8,524,867 from SEQ ID NO: 87 (heavy chain) and SEQ ID NO: 106 (light chain), the relevant disclosures of which are incorporated by reference herein. The heavy chain of this antibody contains the Fc-enhancing S239 and I332E mutations along with mutations that incorporate IgG2 residues into the IgG1 Fc region (K274Q, Y296F, Y300F, L309V, A339T, V397M; SEQ ID NO: 71). For antibody expression, two different plasmids encoding the heavy (SEQ ID NO: 71) and light (SEQ ID NO: 70) chains were transduced into HEK293F cells. Antibodies were purified from cell culture supernatants using protein-A affinity chromatography.

Jurkat-N cells expressing the variant ACTR molecules were mixed at a 1:1 ratio with target Raji cells expressing CD19 and varying concentrations of anti-CD19 antibody (0-133 nM) in a 100-μL reaction volume in RPMI-1640 media supplemented with 10% fetal bovine serum. Reactions were incubated and processed as described in Example 8.

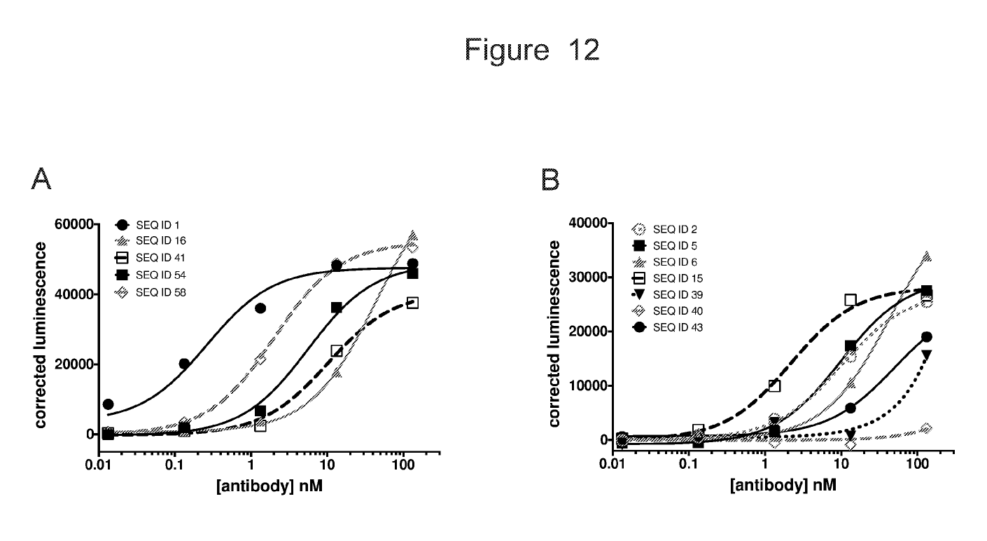

The ability of different ACTR-antibody pairs to activate Jurkat-N cells, as measured by an increase in luminescence, was evaluated for ACTR variants SEQ ID NOs: 2, 5, 6, 15, 16, 39, 40, 41, 43, 54, and 58. These variants all contain amino acid variations within the CD16 region of the ACTR sequence at residues known to be important for CD16-antibody Fc interaction. ACTR variant SEQ ID NO: 1 was also evaluated; this variant has no variations within its CD16 region of the ACTR sequence relative to CD16-V158 sequence (SEQ ID NO: 18). ACTR variants SEQ ID NOs: 1, 2, 5, 6, 16, 41, 43, 54, 58, and 102 all show a dose-dependent increase in luminescence as a function of antibody concentration in the presence of anti-CD19 antibody (FIG. 12). ACTR variant SEQ ID 39 shows some activation at the highest antibody concentration tested and ACTR variant SEQ ID 40 shows no activation at the antibody concentrations tested (FIG. 12, panel B). Corrected luminescence was plotted as a function of antibody concentration and fit to the equation Y=max*X/(EC₅₀+X) where Y is the corrected luminescence, X is the antibody concentration, max is the maximum corrected luminescence, and EC₅₀ is the concentration of antibody that gives the half-maximal response. An EC₅₀ value was determined for ACTR variants SEQ ID NO: 1, SEQ ID NO: 15, and SEQ ID NO: 58; an EC₅₀ could not be determined for the other ACTR variants because the curves were not saturated at the concentrations tested. The EC₅₀ for SEQ ID NO: 1 was 0.26 nM, for SEQ ID NO: 15 was 2.2 nM, and for SEQ ID NO: 58 was 2.0 nM. These three variants showed the most robust activity with the anti-CD19 antibody (FIG. 12, panel A and panel B). ACTR variants SEQ ID NO: 2, SEQ ID NO: 5, SEQ ID NO: 6, SEQ ID NO: 16, SEQ ID NO: 41, and SEQ ID NO: 54 showed a concentration-response that was shifted to higher concentrations relative to that seen with SEQ ID NO: 15 and SEQ ID NO: 22 (FIG. 12, panel A and panel B). The response was shifted to even higher concentrations with ACTR variants SEQ ID NO: 39 and SEQ ID NO: 43 (FIG. 12, panel B). These results demonstrate that ACTR variants that have low or no activity with unmodified antibody variants like rituximab show activity in the presence of an anti-CD19 antibody with Fc-enhancing mutations.

### Example 10: Restoration of ACTR Variant Activity with an Afucosylated Anti-Her2 Antibody

Gamma-retrovirus was generated that encoded ACTR variants SEQ ID NOs: 1, 5, 6, and 7. These viruses were used to infect primary human T-cells to generate cells that express ACTR variants SEQ ID NOs: 1, 5, 6, and 7 on their cell surface. Afucosylated trastuzumab was generated by transfecting two different plasmids encoding the heavy (SEQ ID NO: 78) and light (SEQ ID NO: 75) chains of the antibody into HEK293F cells. Cells were grown in the presence of 2F-peracetyl-fucose (Calbiochem; San Diego, Calif.), which is a fucosylation inhibitor. Afucosylated antibodies are known to mediate tighter binding to the CD16 Fc receptor when compared to their fucosylated counterparts (Shields et al, J. Biol. Chem. (2002) 277:26733-40). Antibody was purified from cell culture supernatants using protein-A affinity chromatography.

ACTR T-cells were used in cytotoxicity assays with Her2-positive SKBR3 target cells that constitutively expressed firefly luciferase and Her2-targeting trastuzumab (Genentech) or afucosylated trastuzumab. T-cells (effector; E) and SKBR3 target cells (target; T) were incubated at a 4:1 effector-to-target ratio (30,000 target cells; 120,000 effector cells) in the presence of different concentrations of trastuzumab or afucosylated trastuzumab (0-2000 nM) in a 200-μL reaction volume in RPMI 1640 media supplemented with 10% fetal bovine serum. Reactions were incubated in a CO₂ (5%) incubator at 37° C. for 24 hr. Cells were pelleted by centrifugation and 100 μL of the supernatant was removed and frozen at −20° C. for subsequent analysis of cytokines. A 100-μL volume of BrightLite Plus luciferase assay reagent (Perkin Elmer) was added to the cells and incubated at room temperature for 10 minutes. Luminescence was measured using an Envision multilabel reader (PerkinElmer; Waltham, Mass.). The percentage of live cells was determined by dividing the luminescence signal of a given sample by the luminescence signal in the absence of antibody for each T-cell type and multiplying by 100. The percent cytotoxicity was determined by subtracting the percent live cells from 100.

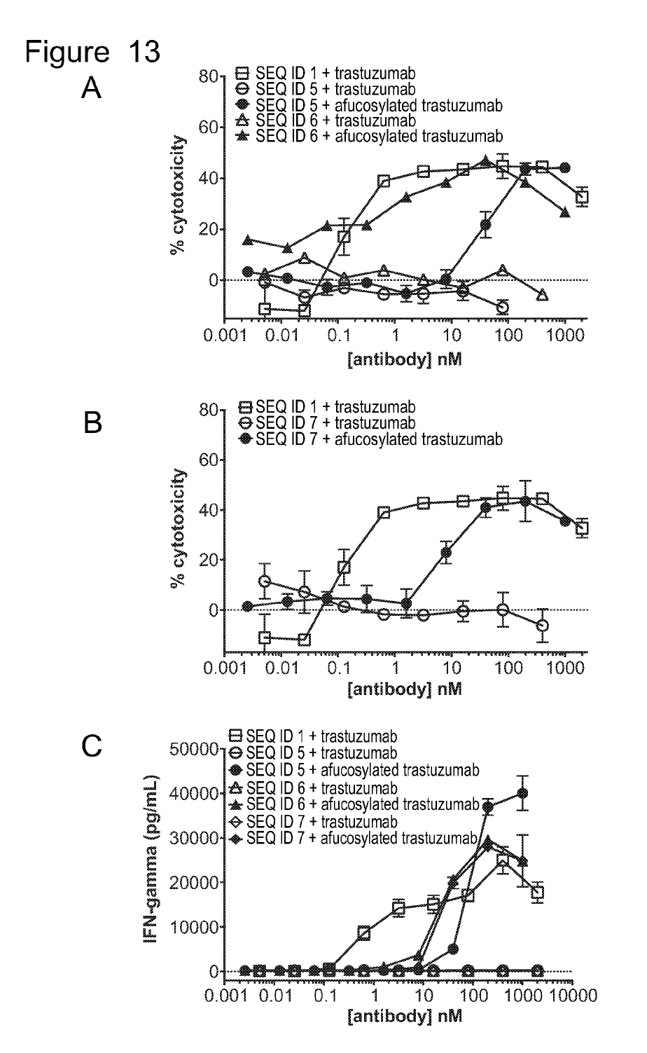

ACTR variant SEQ ID NO: 1 demonstrated a dose-dependent increase in cytotoxicity as a function of trastuzumab concentration (FIG. 13, panels A and B). ACTR variants SEQ ID NOs: 5, 6, and 7 showed no detectable cytotoxicity in the presence of trastuzumab (FIG. 13, panels A and B). In the presence of afucosylated trastuzumab, ACTR variants SEQ ID NOs: 5, 6, and 7 all showed a dose-dependent increase in cytotoxicity as a function of antibody concentration (FIG. 13, panels A and B); ACTR variant SEQ ID NO: 6 showed a dose-response similar to that observed with ACTR variant SEQ ID NO: 1 and trastuzumab (FIG. 13, panel A).

Previously-frozen supernatants were analyzed for IFNγ and raw data was processed as described in Example 7. ACTR variant SEQ ID NO: 1 demonstrated a dose-dependent increase in IFNγ as a function of trastuzumab concentration (FIG. 13, panel C). There was no antibody concentration-dependent increase in IFNγ levels with ACTR variants SEQ ID NO: 5, SEQ ID NO: 6, or SEQ ID NO: 7 in the presence of trastuzumab (FIG. 13, panel C). ACTR variants SEQ ID NO: 5, SEQ ID NO: 6, and SEQ ID NO: 7 demonstrated a dose-dependent increase in IFNγ as a function of afucosylated trastuzumab concentration.

These results demonstrate that ACTR variants that have low or no activity with unmodified antibody variants like trastuzumab show activity in the presence of afucosylated trastuzumab; the afucosylation modification is predicted to allow antibodies to interact more tightly with CD16.

### Example 11: Restoration of ACTR Variant Activity with Afucosylated and Fc-Enhanced Antibodies in Jurkat Activation Assays

Jurkat-N cells were generated as described in Example 8. Gamma-retroviruses encoding ACTR variants shown in Table 8 were each transduced into Jurkat N cells. Afucosylated rituximab was generated by transfecting two different plasmids encoding the heavy and light chains of the antibody into HEK293F cells. Cells were grown in the presence of 2F-peracetyl-fucose (Calbiochem; San Diego, Calif.), which is a fucosylation inhibitor. Afucosylated antibodies are known to mediate tighter binding to the CD16 Fc receptor when compared to their fucosylated counterparts (Shields et al, J. Biol. Chem. (2002) 277:26733-40). Rituximab with Fc-enhancing mutations were generated and purified as described in Example 7.

Jurkat-N cells expressing the variant ACTR molecules were mixed at a 1:1 ratio with target Raji cells expressing CD20 and varying concentrations of antibodies (0-333 nM) in a 100-μL reaction volume in RPMI-1640 media supplemented with 10% fetal bovine serum. Reactions were incubated for 5 hr in a CO₂ (5%) incubator at 37° C. Bright-Glo reagent (100 μL, Promega; Madison, Wis.) was added to lyse the cells and add the luciferin reagent. Reactions were incubated and processed as described in Example 8.

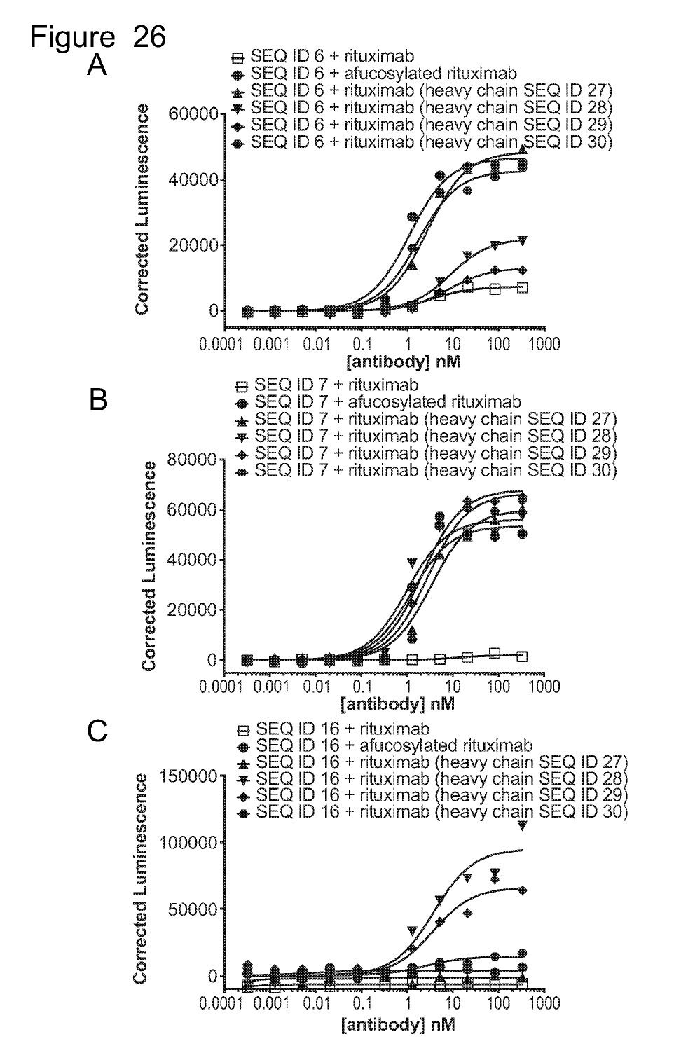

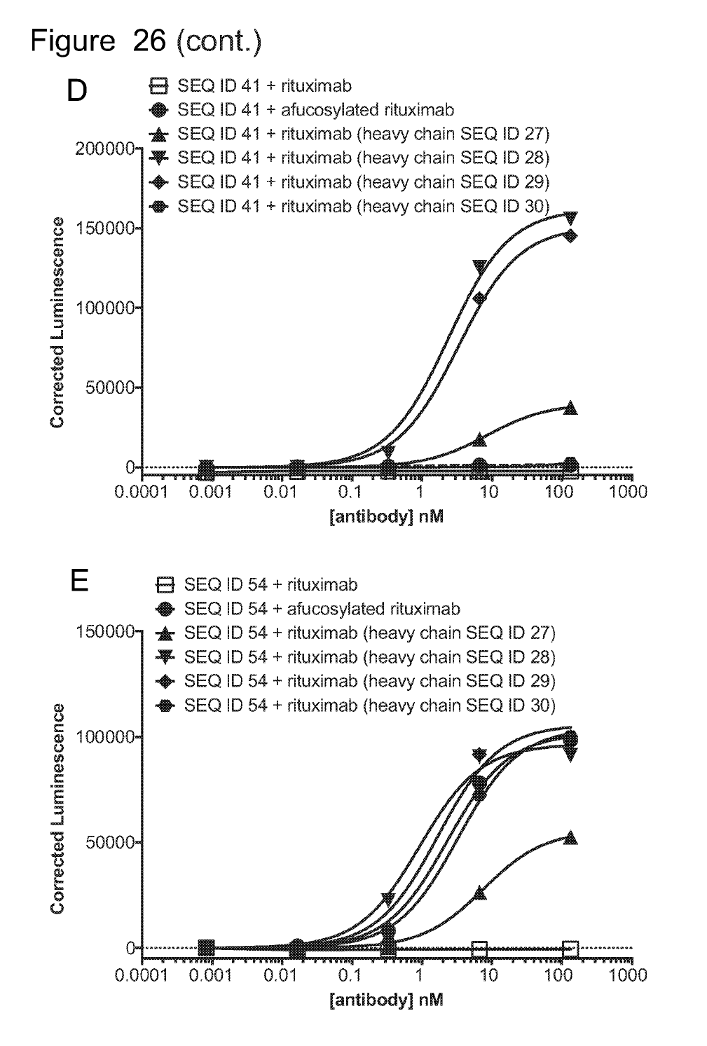

ACTR variants SEQ ID NOs: 6, 7, 16, 41, and 54 all show low or no activity in the presence of rituximab at the antibody concentrations tested (FIG. 26). ACTR variant SEQ ID NO: 6 shows a concentration-dependent increase in luminescence as a function of antibody concentration in the presence of afucosylated rituximab, rituximab with heavy chain variant SEQ ID NO: 27, and rituximab with heavy chain variant SEQ ID NO: 30 (FIG. 26, panel A); ACTR variant SEQ ID NO: 6 shows some activity in the presence of rituximab with heavy chain variant SEQ ID NO: 28 and rituximab with heavy chain variant SEQ ID NO: 29, but at higher antibody concentrations (FIG. 26, panel A). ACTR variant SEQ ID NO: 7 shows a concentration-dependent increase in luminescence as a function of antibody concentration in the presence of afucosylated rituximab, rituximab with heavy chain variant SEQ ID NO: 27, rituximab with heavy chain variant SEQ ID NO: 28, rituximab with heavy chain variant SEQ ID NO: 29, and rituximab with heavy chain variant SEQ ID NO: 30 and shows a similar concentration-response with all of these antibody variants (FIG. 26, panel B). ACTR variant SEQ ID NO: 16 shows a concentration-dependent increase in luminescence as a function of antibody concentration in the presence of rituximab with heavy chain variant SEQ ID NO: 28 and rituximab with heavy chain variant SEQ ID NO: 29 (FIG. 26, panel A); ACTR variant SEQ ID NO: 16 shows a minor amount activity in the presence of rituximab with heavy chain variant SEQ ID NO: 30 at the highest antibodies concentration tested and no activity with rituximab, afucosylated rituximab or rituximab with heavy chain variant SEQ ID NO: 27 (FIG. 26, panel C). ACTR variant SEQ ID NO: 41 shows a concentration-dependent increase in luminescence as a function of antibody concentration in the presence of rituximab with heavy chain variant SEQ ID NO: 28 and rituximab with heavy chain variant SEQ ID NO: 29 (FIG. 26, panel D); ACTR variant SEQ ID NO: 16 shows a small amount activity in the presence of rituximab with heavy chain variant SEQ ID NO: 27 at the higher antibodies concentrations tested and no activity with rituximab, afucosylated rituximab or rituximab with heavy chain variant SEQ ID NO: 30 (FIG. 26, panel D). ACTR variant SEQ ID NO: 54 shows a concentration-dependent increase in luminescence as a function of antibody concentration in the presence of afucosylated rituximab, rituximab with heavy chain variant SEQ ID NO: 28, rituximab with heavy chain variant SEQ ID NO: 29, and rituximab with heavy chain variant SEQ ID NO: 30 (FIG. 26, panel E); ACTR variant SEQ ID NO: 54 shows some activity in the presence of rituximab with heavy chain variant SEQ ID NO:27, but at higher antibody concentrations (FIG. 26, panel E).

Representative EC₅₀ and maximal corrected luminescence values for the ACTR variants tested with Fc-enhanced antibodies are shown in Table 8. Each ACTR variant Jurkat NFAT reporter cell line showed varying maximal luminescence values. For curves that were saturated or showed a robust concentration-response, EC₅₀ and maximal luminescence values are reported from the curve fit. For ACTR-antibody pairs that showed some increase in luminescence over baseline but for which the concentration-response was not saturated, an EC₅₀ value could not be calculated and is not reported (NA is not applicable) and the maximum luminescence value represents the luminescence observed at the highest antibody tested for the experiment. For ACTR-antibody pairs that showed no luminescence above baseline, EC₅₀ and maximum luminescence values could not be calculated and this is indicated as NA in the table. Nearly all ACTR variants tested demonstrated low or no activity in the presence of rituximab (Table 8). ACTR variant SEQ ID NO: 57 showed the most robust activity of the variants in the presence of rituximab, but showed a higher EC₅₀ than that observed with ACTR variant SEQ ID NO: 1 and rituximab. ACTR variants SEQ ID NOs: 3, 8, 33, 34, 35, 36, 44, 45, 46, 47, 48, 49, 50, 51, 52, 58, 59, 60, 61, 62, 63, 64, 65, 66, 67, 68, and 69 showed no restoration of activity with any of the Fc-enhanced variants at the antibody concentrations tested. ACTR variants SEQ ID NOs: 2, 5, 6, 7, 15, 16, 41, 42, 43, 54, 55, 56, and 57 showed a concentration-dependent increase in luminescence with a measurable EC₅₀ in the presence of one or more Fc-enhanced antibody. ACTR variants SEQ ID NOs: 37, 38, 39, 40, and 53 show some activity in the presence of one or more Fc-enhanced antibody at the highest antibody concentrations tested.

TABLE 8
Representative EC50 and maximum signal for activation of ACTR variants in
Jurkat cells in the presence of rituximab and Fc-enhanced rituximab variants

| | maximum | Value: max | | afuco- | | | | |
|---|---|---|---|---|---|---|---|---|
| ACTR | [antibody] | (luminescence | | sylated | | | | |
| SEQ | tested | units), EC₅₀ | wild- | wild- | SEQ | SEQ | SEQ | SEQ |
| ID | (nM) | (nM) | type | type | ID 27 | ID 28 | ID 29 | ID 30 |
| 1 | 333 | max | 45105 | ND | ND | ND | ND | ND |
| | | EC₅₀ | 0.65 | ND | ND | ND | ND | ND |
| 2 | 333 | max | NA | 23760 | 7080 | 50081 | 39166 | 45877 |
| | | EC₅₀ | NA | 3.47 | NA | 3.97 | 4.79 | 5.02 |
| 3 | 333 | max | NA | NA | NA | NA | NA | NA |
| | | EC₅₀ | NA | NA | NA | NA | NA | NA |
| 5 | 333 | max | NA | 50476 | 38594 | 13279 | 5440 | 53374 |
| | | EC₅₀ | NA | 2.63 | 3.79 | 6.81 | NA | 2.88 |
| 6 | 333 | max | 7120 | 46532 | 48495 | 22188 | 13088 | 42704 |
| | | EC₅₀ | NA | 1.13 | 2.71 | 8.82 | 7.96 | 1.73 |
| 7 | 333 | max | NA | 53555 | 59946 | 56114 | 67994 | 66528 |
| | | EC₅₀ | NA | 1.25 | 3.44 | 1.01 | 2.14 | 2.85 |
| 8 | 333 | max | NA | NA | NA | NA | NA | NA |
| | | EC₅₀ | NA | NA | NA | NA | NA | NA |
| 15 | 333 | max | NA | 67725 | 29236 | 133949 | 113855 | 95513 |
| | | EC₅₀ | NA | 2.36 | 4.43 | 0.13 | 0.29 | 1.85 |
| 16 | 333 | max | NA | NA | NA | 95063 | 66145 | 16753 |
| | | EC₅₀ | NA | NA | NA | 3.81 | 3.65 | NA |
| 33 | 133 | max | NA | NA | NA | NA | NA | NA |
| | | EC₅₀ | NA | NA | NA | NA | NA | NA |
| 34 | 133 | max | NA | NA | NA | NA | NA | NA |
| | | EC₅₀ | NA | NA | NA | NA | NA | NA |
| 35 | 133 | max | NA | NA | NA | NA | NA | NA |
| | | EC₅₀ | NA | NA | NA | NA | NA | NA |
| 36 | 133 | max | NA | NA | NA | NA | NA | NA |
| | | EC₅₀ | NA | NA | NA | NA | NA | NA |
| 37 | 133 | max | NA | 2890 | 631 | 426 | 1200 | 7869 |
| | | EC₅₀ | NA | NA | NA | NA | NA | NA |
| 38 | 133 | max | NA | 7560 | NA | NA | NA | 14080 |
| | | EC₅₀ | NA | NA | NA | NA | NA | NA |
| 39 | 133 | max | NA | NA | NA | 19360 | 19760 | NA |
| | | EC₅₀ | NA | NA | NA | NA | NA | NA |
| 40 | 133 | max | NA | 4200 | 5160 | 11640 | 14240 | 8960 |
| | | EC₅₀ | NA | NA | NA | NA | NA | NA |
| 41 | 133 | max | NA | NA | 2920 | 161991 | 150921 | NA |
| | | EC₅₀ | NA | NA | NA | 2.40 | 3.30 | NA |
| 42 | 133 | max | NA | NA | 47640 | 101672 | 113013 | NA |
| | | EC₅₀ | NA | NA | NA | 1.86 | 2.21 | NA |
| 43 | 133 | max | NA | NA | NA | 89343 | 84881 | NA |
| | | EC₅₀ | NA | NA | NA | 4.62 | 6.96 | NA |
| 44 | 133 | max | NA | NA | NA | NA | NA | NA |
| | | EC₅₀ | NA | NA | NA | NA | NA | NA |
| 45 | 133 | max | NA | NA | NA | NA | NA | NA |
| | | EC₅₀ | NA | NA | NA | NA | NA | NA |
| 46 | 133 | max | NA | NA | NA | NA | NA | NA |
| | | EC₅₀ | NA | NA | NA | NA | NA | NA |
| 47 | 133 | max | NA | NA | NA | NA | NA | NA |
| | | EC₅₀ | NA | NA | NA | NA | NA | NA |
| 48 | 133 | max | NA | NA | NA | NA | NA | NA |
| | | EC₅₀ | NA | NA | NA | NA | NA | NA |
| 49 | 133 | max | NA | NA | NA | NA | NA | NA |
| | | EC₅₀ | NA | NA | NA | NA | NA | NA |
| 50 | 133 | max | NA | NA | NA | NA | NA | NA |
| | | EC₅₀ | NA | NA | NA | NA | NA | NA |
| 51 | 133 | max | NA | NA | NA | NA | NA | NA |
| | | EC₅₀ | NA | NA | NA | NA | NA | NA |
| 52 | 133 | max | NA | NA | NA | NA | NA | NA |
| | | EC₅₀ | NA | NA | NA | NA | NA | NA |
| 53 | 133 | max | NA | NA | NA | 15760 | 11560 | NA |
| | | EC₅₀ | NA | NA | NA | NA | NA | NA |
| 54 | 133 | max | NA | 101678 | 55617 | 96447 | 105883 | 104069 |
| | | EC₅₀ | NA | 2.32 | 7.52 | 0.93 | 1.69 | 3.28 |
| 55 | 133 | max | 45767 | 67000 | 117621 | 111641 | 112935 | 81000 |
| | | EC₅₀ | NA | NA | 2.07 | 0.39 | 0.94 | NA |
| 56 | 133 | max | 26480 | 37560 | 93660 | 84504 | 77833 | 62720 |
| | | EC₅₀ | NA | NA | 3.39 | 3.46 | 3.38 | NA |
| 57 | 133 | max | 62493 | 68077 | 78273 | 69702 | 70610 | 73811 |
| | | EC₅₀ | 1.55 | 1.78 | 0.41 | 1.07 | 0.55 | 1.39 |
| 58 | 133 | max | NA | NA | NA | NA | NA | NA |
| | | EC₅₀ | NA | NA | NA | NA | NA | NA |
| 59 | 133 | max | NA | NA | NA | NA | NA | NA |
| | | EC₅₀ | NA | NA | NA | NA | NA | NA |
| 60 | 133 | max | NA | NA | NA | NA | NA | NA |
| | | EC₅₀ | NA | NA | NA | NA | NA | NA |
| 61 | 133 | max | NA | NA | NA | NA | NA | NA |
| | | EC₅₀ | NA | NA | NA | NA | NA | NA |
| 62 | 133 | max | NA | NA | NA | NA | NA | NA |
| | | EC₅₀ | NA | NA | NA | NA | NA | NA |
| 63 | 133 | max | NA | NA | NA | NA | NA | NA |
| | | EC₅₀ | NA | NA | NA | NA | NA | NA |
| 64 | 133 | max | NA | NA | NA | NA | NA | NA |
| | | EC₅₀ | NA | NA | NA | NA | NA | NA |
| 65 | 133 | max | NA | NA | NA | NA | NA | NA |
| | | EC₅₀ | NA | NA | NA | NA | NA | NA |
| 66 | 133 | max | NA | NA | NA | NA | NA | NA |
| | | EC₅₀ | NA | NA | NA | NA | NA | NA |
| 67 | 133 | max | NA | NA | NA | NA | NA | NA |
| | | EC₅₀ | NA | NA | NA | NA | NA | NA |
| 68 | 133 | max | NA | NA | NA | NA | NA | NA |
| | | EC₅₀ | NA | NA | NA | NA | NA | NA |
| 69 | 133 | max | NA | NA | NA | NA | NA | NA |
| | | EC₅₀ | NA | NA | NA | NA | NA | NA |

ND = not determined
NA = not applicable

ACTR-mediated activation of Jurkat NFAT cells was also evaluated in the presence of afucosylated Fc-enhanced antibodies. For these experiments, afucosylated rituximab with heavy chain variant SEQ ID NO: 28 was generated by transfecting two different plasmids encoding the rituximab light chain and the heavy chain SEQ ID NO: 28 into HEK293F cells. Cells were grown in the presence of 2F-peracetyl-fucose (Calbiochem; San Diego, Calif.), which is a fucosylation inhibitor. Antibody was purified from cell culture supernatants using protein-A affinity chromatography. Experiments were carried out as described above with ACTR variants SEQ ID NOs: 5, 6, 15, 16, 41, 54, and 58 with rituximab, afucosylated rituximab, rituximab with heavy chain variant SEQ ID NO: 28, and afucosylated rituximab with heavy chain variant SEQ ID NO: 28.

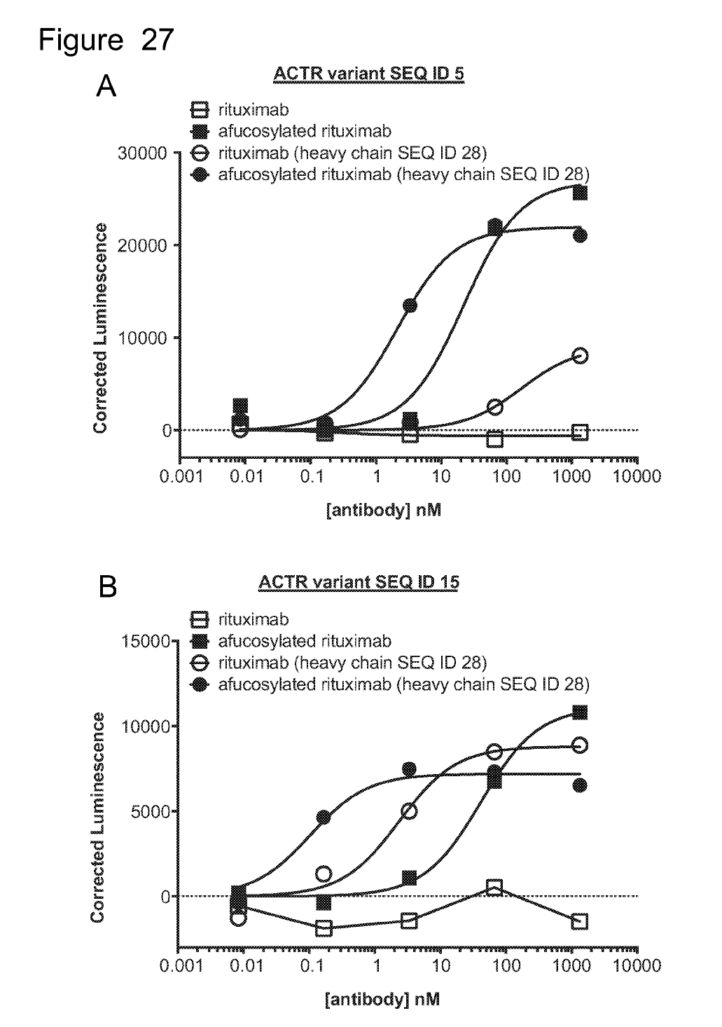

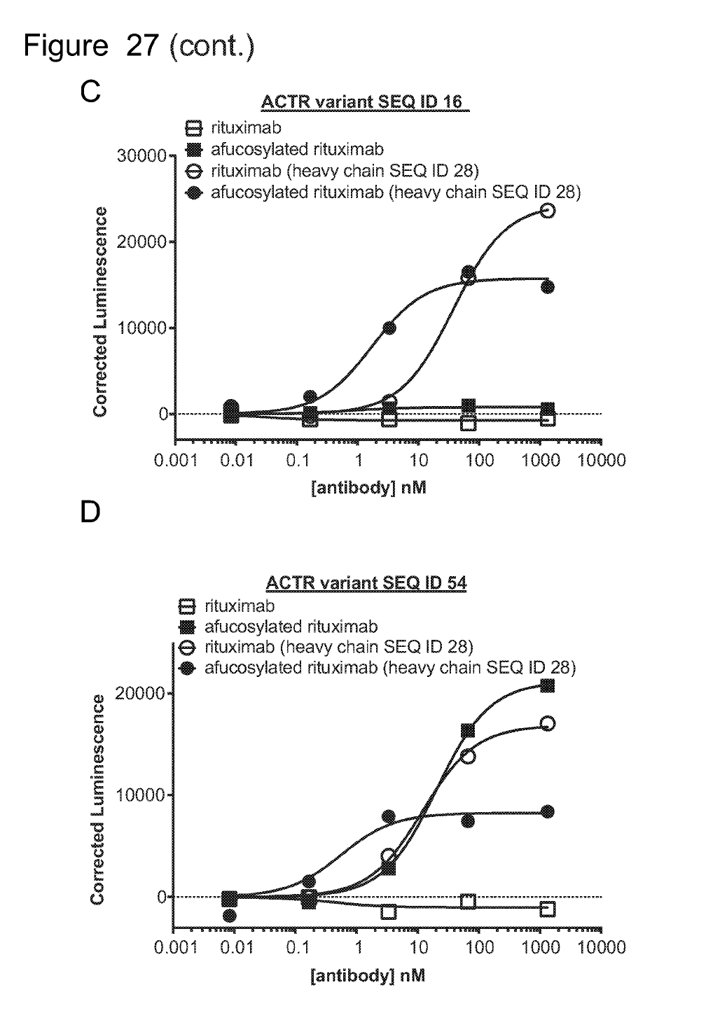

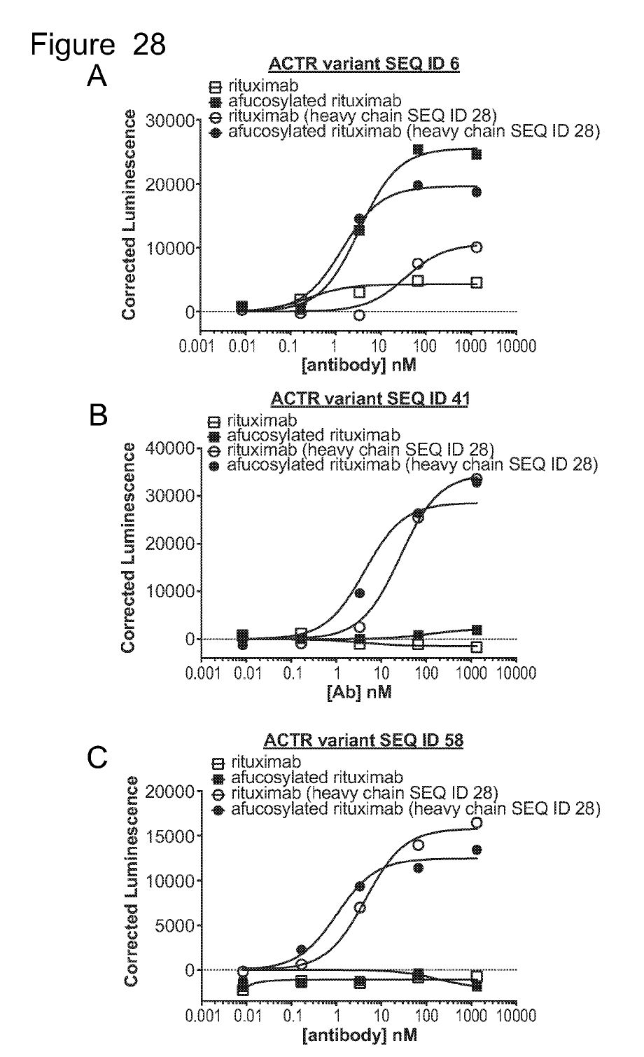

ACTR variants SEQ ID NOs: 5, 15, 16, and 54 showed enhanced activity, as evidenced by a lower concentration-response as a function of antibody concentration, with afucosylated rituximab with heavy chain variant SEQ ID NO: 28 relative to afucosylated rituximab or rituximab with heavy chain variant SEQ ID NO: 28 (FIG. 27). These results suggest that the afucosylation modification and the Fc-amino-acid modifications in SEQ ID NO: 28 act together, either additively or synergistically, with these ACTR variants. ACTR variants SEQ ID NOs: 6, 41, and 58 did not show enhanced activity as a function of antibody concentration with afucosylated rituximab with heavy chain variant SEQ ID NO: 28 over that observed with afucosylated rituximab (ACTR variant SEQ ID NO: 6) or rituximab with heavy chain variant SEQ ID NO: 28 (ACTR variants SEQ ID NOs: 41 and 58) (FIG. 28). ACTR variants SEQ ID NO: 41 and SEQ ID NO: 58 both contain the N164Q mutation which is expected to be relatively insensitive to the enhanced activity with afucosylated antibodies gained by variants without this mutation (Ferrara et al., J. Biol. Chem. (2006) 81: 5032-36).

ACTR-mediated activation of Jurkat NFAT cells was also evaluated in the presence of other mutant heavy chain antibodies. For these experiments, rituximab with heavy chain variant SEQ ID NO: 76 and rituximab with heavy chain variant SEQ ID NO: 77 were generated by transfecting two different plasmids encoding the rituximab light chain and the appropriate heavy chain into HEK293F cells. Antibody was purified from cell culture supernatants using protein-A affinity chromatography. Experiments were carried out as described above with ACTR variants SEQ ID NOs: 2, 42, and 43 with rituximab with heavy chain variant SEQ ID NO: 29, rituximab with heavy chain variant SEQ ID NO: 76, and rituximab with heavy chain variant SEQ ID NO: 77.

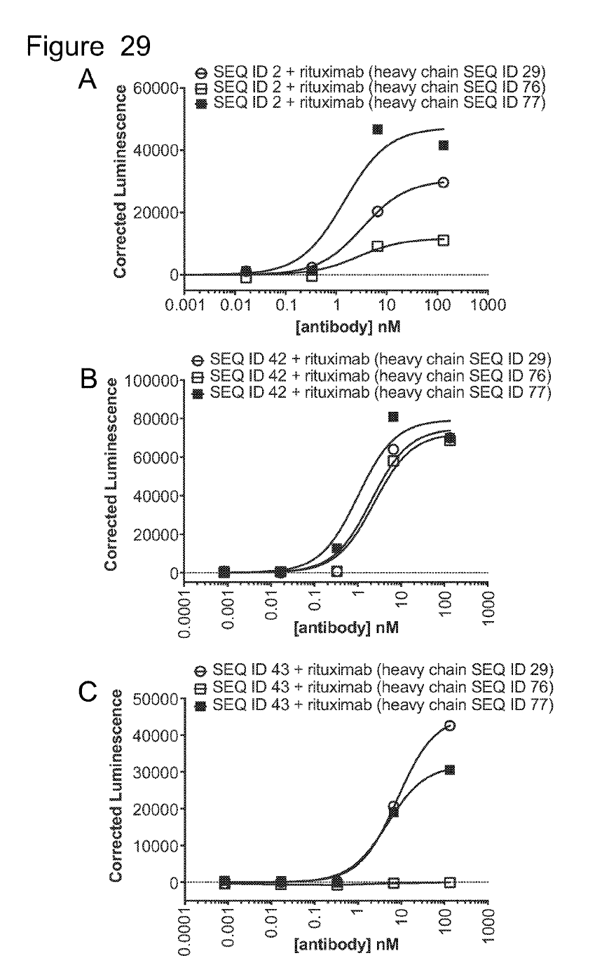

ACTR variants SEQ ID NOs: 2, 42, and 43 showed a concentration-dependent increase in activity as a function of rituximab with heavy chain variant SEQ ID NO: 77 (FIG. 29). The activity of ACTR variant SEQ ID NO: 2 with rituximab with heavy chain variant SEQ ID NO: 77 had a lower antibody concentration-response relative to that observed with rituximab with heavy chain variant SEQ ID NO: 29 (FIG. 29, panel A); ACTR variants SEQ ID NOs: 42 and 43 had a similar concentration-response with both antibodies (FIG. 29, panels B and C). ACTR variants SEQ ID NOs: 2 and 42 showed a concentration-dependent increase in activity as a function of rituximab with heavy chain variant SEQ ID NO: 76 (FIG. 29). The activity of ACTR variant SEQ ID NO: 2 with rituximab with heavy chain variant SEQ ID NO: 76 had a higher antibody concentration-response relative to that observed with rituximab with heavy chain variant SEQ ID NO: 29 (FIG. 29, panel A); ACTR variant SEQ ID NO: 42 had a similar concentration-response with all three antibodies (FIG. 29, panel B). ACTR variant SEQ ID NO: 43 showed no activity in the presence of rituximab with heavy chain variant SEQ ID NO: 76 at the antibody concentrations tested (FIG. 29, panel C).

These experiments demonstrate that specific mutations or combinations of mutations in the CD16 region of ACTR result in a loss of interaction with unmodified rituximab. The activity of many ACTR variants is restored in the presence of antibodies that are afucosylated or have Fc-enhancing amino acid mutations. In some cases, the combination of afucosylation and Fc-enhancing amino acids results in additive or synergistic restoration of ACTR variant activity. These experiments demonstrate the activity of ACTR variants that have impaired activity in the presence of wild-type rituximab can be restored with modified antibodies.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one of skill in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A chimeric receptor, comprising:
(a) an extracellular domain, which is either a mutated extracellular ligand-binding domain of an Fc receptor, or a single chain antibody fragment; and
(b) a cytoplasmic signaling domain,
wherein, as compared with the wild-type counterpart, the mutated extracellular ligand-binding domain of the Fc receptor comprises a mutation at one or more residues involved in Fc receptor/Fc interaction such that the mutated extracellular ligand-binding domain of the Fc receptor has a reduced binding activity to a wild-type Fc fragment relative to the wild-type counterpart; and
wherein the single chain antibody fragment binds preferentially to a mutated Fc fragment as relative to its wild-type counterpart.

2. The chimeric receptor of claim 1, wherein the extracellular domain is the mutated extracellular ligand-binding domain of an Fc receptor.

3. The chimeric receptor of claim 1, wherein the chimeric receptor further comprises one or more additional domains selected from the group consisting of:
a transmembrane domain;
one or more co-stimulatory signaling domains; and
a hinge domain.

4. The chimeric receptor of claim 3, wherein the chimeric receptor comprises, from N terminus to C terminus,
(a) the extracellular domain;
(b) the transmembrane domain;
(c) the one or more co-stimulatory signaling domains; and
(d) the cytoplasmic signaling domain.

5. The chimeric receptor of 4, further comprising the hinge domain, which is located between (a) and (b).

6. The chimeric receptor of claim 1, wherein the chimeric receptor further comprises a signal peptide.

7. The chimeric receptor of any one of claim 1, wherein the Fc receptor is an Fey receptor (FcγR).

8. The chimeric receptor of claim 7, wherein the FcγR is selected from the group consisting of CD16A, CD16B, CD64A, CD64B, CD64C, CD32A, and CD32B.

9. The chimeric receptor of claim 7, wherein the one or more residues where the mutation occurs are located in the D2 domain of the extracellular ligand-binding domain of the FcγR.

10. The chimeric receptor of claim 7, wherein the FcγR is CD16A.

11. The chimeric receptor of claim 10, wherein the mutation is an amino acid substitution at one or more positions corresponding to 92, 122, 134, 136, 160, 161, 163, and 164 in SEQ ID NO: 18.

12. The chimeric receptor of claim 10, wherein the mutated extracellular ligand-binding domain of the Fc receptor comprises amino acid substitutions at two or more positions selected from the group consisting of the positions corresponding to 92, 122, 134, 136, 160, 161, 163, and 164 in SEQ ID NO: 18.

13. The chimeric receptor of claim 10, wherein the mutated extracellular ligand-binding domain of the Fc receptor comprises an amino acid substitution at a position corresponding to 160 in SEQ ID NO:18, at a position corresponding to 134 in SEQ ID NO:18, at a position corresponding to 122 in SEQ ID NO: 18, at a position corresponding to 164 in SEQ ID NO:18, or a combination thereof.

14. The chimeric receptor of claim 13, wherein the mutated extracellular ligand-binding domain of the Fc receptor comprises a V to Q or a V to W amino acid substitution at the position corresponding to 160 in SEQ ID NO: 18.

15. The chimeric receptor of claim 13, wherein the mutated extracellular ligand-binding domain of the Fc receptor comprises a Y to A amino acid substitution at the position corresponding to 134 in SEQ ID NO: 18.

16. The chimeric receptor of claim 13, wherein the mutated extracellular ligand-binding domain of the Fc receptor comprises a K to L amino acid substitution at the position corresponding to 122 in SEQ ID NO: 18.

17. The chimeric receptor of claim 13, wherein the mutated extracellular ligand-binding domain of the Fc receptor comprises an N to Q amino acid substitution at the position corresponding to 164 in SEQ ID NO: 18.

18. The chimeric receptor of claim 10, wherein the mutated extracellular ligand-binding domain of the Fc receptor is a mutated CD16A selected from the group consisting of mutant V160Q, mutant V160W, mutant Y134A, mutant K122L, and mutant Y134A/N164Q.

19. The chimeric receptor of claim 1, wherein the cytoplasmic signaling domain is a cytoplasmic signaling domain of CD3ζ.

20. The chimeric receptor of claim 1, which comprises an amino acid sequence selected from the group consisting of the amino acid sequence of SEQ ID NO: 1-16 and 33-69.

21. A nucleic acid comprising a nucleotide sequence encoding a chimeric receptor of claim 1.

22. A vector comprising the nucleic acid of claim 21.

23. The vector of claim 22, wherein the vector is a viral vector or a transposon.

24. The vector of claim 23, wherein the viral vector is a retroviral vector, a lentiviral vector, or an adeno-associated viral vector.

25. An immune cell expressing the chimeric receptor of claim 1.

26. The immune cell of claim 25, which is a T lymphocyte or a natural killer (NK) cell.

27. The immune cell of claim 26, wherein the T lymphocyte or the NK cell is activated and/or expanded ex vivo.

28. The immune cell of claim 27, wherein the T lymphocyte or the NK cell is a T lymphocyte or an NK cell isolated from a patient having cancer.

29. The immune cell of claim 21, wherein in the T lymphocyte, the expression of the endogenous T cell receptor has been inhibited or eliminated.

30. A pharmaceutical composition comprising
An immune cell that expresses the chimeric receptor of claim 1 and a pharmaceutically acceptable carrier or excipient.

31. The pharmaceutical composition of claim 30, further comprising an Fc-containing polypeptide that binds to the chimeric receptor.

32. The pharmaceutical composition of claim 31, wherein the Fc-containing polypeptide is an antibody.

33. The pharmaceutical composition of claim 31, wherein the Fc-containing polypeptide is afucosylated in its Fc domain.

34. The pharmaceutical composition of claim 31, wherein the Fc-containing polypeptide comprises one or more mutations in the Fc region such that the mutated Fc-containing polypeptide has an enhanced binding activity to the chimeric receptor as compared with its wild-type counterpart.

35. The pharmaceutical composition of claim 33, wherein the Fc-containing polypeptide is an antibody containing an amino acid substitution at one or more positions corresponding to S239, F243, R292, S298, Y300, V305, A330, I332, E333, K334, and P396 of a wild-type antibody, wherein the numbering is according to the EU index.

36. The pharmaceutical composition of claim 35, wherein the amino acid substitution is S239D, F243L, R292P, S298A, Y300L, V305I, A330L, I332E, I332D, E333A, K334A, P396L, or a combination thereof.

37. The pharmaceutical composition of claim 31, wherein the immune cell expresses a chimeric receptor, which comprises an amino acid substitution at one or more positions corresponding to 122, 134, 160, and 164 in SEQ ID NO: 18, and wherein the Fc-containing polypeptide is afucosylated in its Fc domain.

38. The pharmaceutical composition of claim 37, wherein the immune cell expresses a chimeric receptor, which comprises the CD16A mutant V160Q, the CD16A mutant V160W, or the CD16A mutant K122L, and wherein the Fc-containing polypeptide is an afucosylated full-length antibody.

39. The pharmaceutical composition of claim 31, wherein the immune cell expresses a chimeric receptor which comprises an amino acid substitution at one or more positions corresponding to 122, 134, 160, and 164 in SEQ ID NO: 18, and wherein the Fc-containing polypeptide is an antibody containing an amino acid substitution at one or more positions corresponding to S239, F243, R292, S298, Y300, V305, A330, I332, E333, K334, and P396 of a wild-type antibody.

40. The pharmaceutical composition of claim 39, wherein the immune cell expresses a chimeric receptor, which comprises the CD16A mutant Y134A/N164Q, and wherein the Fc-containing polypeptide is an antibody containing (i) S239D, A330L, and I332E substitutions, or (ii) S239D and I332E substitutions as compared with the wild-type counterpart.

41. The pharmaceutical composition of claim 39, wherein the immune cell expresses a chimeric receptor, which comprises the CD16A mutant Y134A, and wherein the Fc-containing polypeptide is an antibody containing (i) S239D, A330L, and I332E substitutions, or (ii) S239D and I332E substitutions as compared with the wild-type counterpart.

42. The pharmaceutical composition of claim 39, wherein the immune cell expresses a chimeric receptor, which comprises the CD16A mutant K122L, and wherein the Fc-containing polypeptide is an antibody containing (i) S298A, E333A, and K334A substitutions, or (ii) F243L, R292P, Y300L, V305I, and P396L substitutions as compared with the wild-type counterpart.

43. The pharmaceutical composition of claim 39, wherein the immune cell expresses a chimeric receptor, which comprises the CD16A mutant V160Q, and wherein the Fc-containing polypeptide is an antibody containing (i) S298A, E333A, and K334A substitutions, (ii) S239D, A330L, and I332E substitutions, (iii) S239D and I332E substitutions, or (iv) F243L, R292P, Y300L, V305I, and P396L substitutions as compared with the wild-type counterpart.

44. The pharmaceutical composition of claim 39, wherein the immune cell expresses a chimeric receptor, which comprises the CD16A mutant V160W, and wherein the Fc-containing polypeptide is an antibody containing (i) S298A, E333A, and K334A substitutions, (ii) S239D, A330L, and I332E substitutions, (iii) S239D and I332E substitutions, or (iv) F243L, R292P, Y300L, V305I, and P396L substitutions as compared with the wild-type counterpart.

45. A kit for an antibody-coupled T cell receptor (ACTR) immunotherapy, comprising:
(i) immune cells expressing the chimeric receptor of claim 1; and
(ii) an Fc-containing polypeptide that binds the chimeric receptor.

46. The kit of claim 45, wherein the Fc-containing polypeptide is an antibody.

47. The kit of claim 45, wherein the Fc-containing polypeptide is afucosylated at its Fc domain glycosylation site.

48. The kit of claim 45, wherein the Fc-containing polypeptide comprises one or more mutations in the Fc region therein relative to the wild-type Fc counterpart such that Fc-containing polypeptide has an enhanced binding activity to the chimeric receptor as compared with a wild-type counterpart.

49. The kit of claim 46, wherein the Fc-containing polypeptide is an antibody containing an amino acid substitution at one or more positions corresponding to S239, F243, R292, S298, Y300, V305, A330, I332, E333, K334, and P396 of a wild-type antibody, and wherein the numbering is according to the EU index.

50. The kit of claim 49, wherein the amino acid substitution is S239D, F243L, R292P, S298A, Y300L, V305I, A330L, I332E, I332D, E333A, K334A, P396L, or a combination thereof.

51. The kit of claim 45, wherein the chimeric receptor comprises an amino acid substitution at one or more positions corresponding to 122, 134, 160, and 164 in SEQ ID NO: 18, and wherein the Fc-containing polypeptide is afucosylated in its Fc domain.

52. The kit of claim 51, wherein the immune cell expresses a chimeric receptor, which comprises the CD16A mutant V160Q, the CD16A mutant V160W, or the CD16A mutant K122L, and wherein the Fc-containing polypeptide is an afucosylated full-length antibody.

53. The kit of claim 45, wherein the immune cell expresses a chimeric receptor, which comprises an amino acid substitution at one or more positions corresponding to 122, 134, 160, and 164 in SEQ ID NO: 18, and wherein the Fc-containing polypeptide is an antibody containing an amino acid substitution at one or more positions corresponding to S239, F243, R292, S298, Y300, V305, A330, I332, E333, K334, and P396 of a wild-type antibody.

54. The kit of claim 53, wherein the immune cell expresses a chimeric receptor, which comprises the CD16A mutant Y134A/N164Q, and wherein the Fc-containing polypeptide is an antibody containing (i) S239D, A330L, and I332E substitutions, or (ii) S239D and I332E substitutions as compared with the wild-type counterpart.

55. The kit of claim 53, wherein the immune cell expresses a chimeric receptor, which comprises the CD16A mutant Y134A, and wherein the Fc-containing polypeptide is an antibody containing (i) S239D, A330L, and I332E substitutions, or (ii) S239D and I332E substitutions as compared with the wild-type counterpart.

56. The kit of claim 53, wherein the immune cell expresses a chimeric receptor, which comprises the CD16A mutant K122L, and wherein the Fc-containing polypeptide is an antibody containing (i) S298A, E333A, and K334A substitutions, or (ii) F243L, R292P, Y300L, V305I, and P396L substitutions as compared with the wild-type counterpart.

57. The kit of claim 53, wherein the immune cell expresses a chimeric receptor, which comprises the CD16A mutant V160Q, and wherein the Fc-containing polypeptide is an antibody containing (i) S298A, E333A, and K334A substitutions, (ii) S239D, A330L, and I332E substitutions, (iii) S239D and I332E substitutions, or (iv) F243L, R292P, Y300L, V305I, and P396L substitutions as compared with the wild-type counterpart.

58. The kit of claim 53, wherein the immune cell expresses a chimeric receptor, which comprises the CD16A mutant V160W, and wherein the Fc-containing polypeptide is an antibody containing (i) S298A, E333A, and K334A substitutions, (ii) S239D, A330L, and I332E substitutions, (iii) S239D and I332E substitutions, or (iv) F243L, R292P, Y300L, V305I, and P396L substitutions as compared with the wild-type counterpart.

59. A method for enhancing the efficacy of an antibody-based immunotherapy, the method comprising administering to a subject in need thereof
(i) a therapeutically effective amount of an immune cell that express the chimeric receptor of claim 1, and
(ii) a therapeutically effective amount of an Fc-containing polypeptide that binds the chimeric receptor.

60. The method of claim 59, wherein the Fc-containing polypeptide is an antibody.

61. The method of claim 59, wherein the Fc-containing polypeptide is afucosylated in its Fc domain.

62. The method of claim 59, wherein the Fc-containing polypeptide comprises one or more mutations in the Fc region therein relative to its wild-type counterpart such that it has an enhanced binding activity to the chimeric receptor as compared with the wild-type counterpart.

63. The method of claim 62, wherein the Fc-containing polypeptide is an antibody having an amino acid substitution at one or more positions corresponding to S239, F243, R292, S298, Y300, V305, A330, I332, E333, K334, and P396 of a wild-type antibody, and wherein the numbering system is according to the EU index.

64. The method of claim 63, wherein the amino acid substitution is S239D, F243L, R292P, S298A, Y300L, V305I, A330L, I332E, I332D, E333A, K334A, P396L, or a combination thereof.

65. The method of claim 59, wherein the chimeric receptor comprises an amino acid substitution at one or more positions corresponding to 122, 134, 160, and 164 in SEQ ID NO: 18, and wherein the Fc-containing polypeptide is afucosylated in its Fc domain.

66. The method of claim 65, wherein the immune cell expresses a chimeric receptor, which comprises the CD16A mutant V160Q, the CD16A mutant V160W, or the CD16A mutant K122L, and wherein the Fc-containing polypeptide is an afucosylated full-length antibody.

67. The method of claim 59, wherein the immune cell expresses a chimeric receptor, which comprises an amino acid substitution at one or more positions corresponding to 122, 134, 160, and 164 in SEQ ID NO: 18, and wherein the Fc-containing polypeptide is an antibody containing an amino acid substitution at one or more positions corresponding to S239, F243, R292, S298, Y300, V305, A330, I332, E333, K334, and P396 of a wild-type antibody.

68. The method of claim 67, wherein the immune cell expresses a chimeric receptor, which comprises the CD16A mutant Y134A/N164Q, and wherein the Fc-containing polypeptide is an antibody containing (i) S239D, A330L, and I332E substitutions, or (ii) S239D and I332E substitutions as compared with the wild-type counterpart.

69. The method of claim 67, wherein the immune cell expresses a chimeric receptor, which comprises the CD16A mutant Y134A, and wherein the Fc-containing polypeptide is an antibody containing (i) S239D, A330L, and I332E substitutions, or (ii) S239D and I332E substitutions as compared with the wild-type counterpart.

70. The method of claim 67, wherein the immune cell expresses a chimeric receptor, which comprises the CD16A mutant K122L, and wherein the Fc-containing polypeptide is an antibody containing (i) S298A, E333A, and K334A substitutions, or (ii) F243L, R292P, Y300L, V305I, and P396L substitutions as compared with the wild-type counterpart.

71. The method of claim 67, wherein the immune cell expresses a chimeric receptor, which comprises the CD16A mutant V160Q, and wherein the Fc-containing polypeptide is an antibody containing (i) S298A, E333A, and K334A substitutions, (ii) S239D, A330L, and I332E substitutions, (iii) S239D and I332E substitutions, or (iv) F243L, R292P, Y300L, V305I, and P396L substitutions as compared with the wild-type counterpart.

72. The method of claim 67, wherein the immune cell expresses a chimeric receptor, which comprises the CD16A mutant V160W, and wherein the Fc-containing polypeptide is an antibody containing (i) S298A, E333A, and K334A substitutions, (ii) S239D, A330L, and I332E substitutions, (iii) S239D and I332E substitutions, or (iv) F243L, R292P, Y300L, V305I, and P396L substitutions as compared with the wild-type counterpart.

73. The method of claim 59, wherein the subject has a cancer.

74. The method of claim 73, wherein the cancer is selected from the group consisting of carcinoma, lymphoma, sarcoma, blastoma, and leukemia.

75. The method of claim 73, wherein the cancer is selected from the group consisting of a cancer of B-cell origin, breast cancer, gastric cancer, neuroblastoma, osteosarcoma, lung cancer, melanoma, prostate cancer, colon cancer, renal cell carcinoma, ovarian cancer, rhabdomyo sarcoma, leukemia, and Hodgkin's lymphoma.

76. The method of claim 75, wherein the cancer of B-cell origin is selected from the group consisting of B-lineage acute lymphoblastic leukemia, B-cell chronic lymphocytic leukemia, and B-cell non-Hodgkin's lymphoma.

77. The method of claim 59, wherein the immune cell is a T lymphocyte or an NK cell.

78. The method of claim 77, wherein the T lymphocyte or NK cell is an autologous T lymphocyte or an autologous NK cell isolated from the subject.

79. The method of claim 78, wherein prior to the administration step, the autologous T lymphocyte or autologous NK cells are activated and/or expanded ex vivo.

80. The method of claim 77, wherein the T lymphocyte or NK cell is an allogeneic T lymphocyte or an allogeneic NK cell.

81. The method of claim 80, wherein the allogeneic T lymphocyte is engineered to reduce graft-versus-host effects or host-versus-graft effects.

82. The method of claim 81, wherein the endogenous T cell receptor of the allogeneic T lymphocyte has been inhibited or eliminated.

83. The method of claim 80, wherein prior to the administration step, the allogenic T lymphocyte or allogenic NK cell are activated and/or expanded ex vivo.

84. The method of claim 59, wherein the Fc-containing polypeptide is afucosylated in its Fc domain.

85. The method of claim 84, wherein the Fc-containing polypeptide is a therapeutic antibody.

86. The method of claim 85, wherein the therapeutic antibody is selected from the group consisting of Rituximab, Trastuzumab, hu14.18K322A, Epratuzumab, Cetuximab, and Labetuzumab.

87. A method for preparing an immune cell expressing a chimeric receptor, comprising
(i) providing a population of immune cells;
(ii) introducing into the immune cells a nucleic acid encoding a chimeric receptor of claim 1; and
(iii) culturing the immune cells under conditions allowing for expression of the chimeric receptor.

88. The method of claim 87, wherein the population of immune cells is derived from peripheral blood mononuclear cells (PBMC).

89. The method of claim 87 or 88, wherein the immune cells comprise T lymphocytes and/or NK cells.

90. The method of claim 87, wherein the immune cells are derived from a human cancer patient.

91. The method of claim 87, wherein the nucleic acid is a viral vector or a transposon.

92. The method of claim 91, wherein the viral vector is a lentiviral vector, a retroviral vector, or an adeno-associated viral vector.

93. The method of claim 87, wherein the nucleic acid is an RNA molecule.

94. The method of claim 87, wherein the nucleic acid is introduced into the immune cells by lentiviral transduction, retroviral transduction, adeno-associated viral transduction, DNA electroporation, RNA electroporation, or transposon electroporation.

95. The method of claim 87, further comprising (iv) activating the immune cells expressing the chimeric receptor.

96. The method claim 95, wherein the immune cells comprise T lymphocytes, which are activated in the presence of one or more of anti-CD3 antibody, anti-CD28 antibody, IL-2, IL-7, IL-15, and phytohemoagglutinin.

97. The method of claim 95, wherein the immune cells comprise NK cell, which are activated in the presence of one or more of 4-1BB ligand, anti-4-1BB antibody, IL-15 protein, IL-15 receptor antibody, IL-2 protein, IL-21 protein, K562 cell line.
